(19) United States
(12) Patent Application Publication
(10) Pub. No.: US 2022/0404334 A1
(43) Pub. Date: Dec. 22, 2022

(54) BIOCHIP HAVING MICROCHANNEL PROVIDED WITH CAPTURING AGENT FOR PERFORMING CYTOLOGICAL ANALYSIS

(71) Applicant: CASE WESTERN RESERVE UNIVERSITY, Cleveland, OH (US)

(72) Inventors: UMUT GURKAN, Cleveland, OH (US); ERDEM KUCUKAL, Cleveland, OH (US); UTKU GOREKE, Cleveland, OH (US); YUNCHENG MAN, Cleveland, OH (US)

(21) Appl. No.: 17/773,774

(22) PCT Filed: Oct. 30, 2020
(86) PCT No.: PCT/US2020/058272

§ 371 Date: May 2, 2022

Related U.S. Application Data
(60) Provisional application No. 62/928,109, filed on Oct. 30, 2019.
(60) Provisional application No. 62/989,360, filed on Mar. 13, 2020.
(60) Provisional application No. 63/037,287, filed on Jun. 10, 2020.
(60) Provisional application No. 63/043,536, filed on Jun. 24, 2020.
(60) Provisional application No. 63/049,443, filed on Jul. 8, 2020.
(60) Provisional application No. 63/072,502, filed on Aug. 31, 2020.

(51) Int. Cl. G01N 33/49; B01L 3/00; G01N 33/50

(52) U.S. Cl. CPC G01N 33/4915; B01L 3/502761; G01N 33/5044; G01N 2800/22; B01L 2200/0647; B01L 2300/16; B01L 2300/0877

(57) ABSTRACT

A microfluidic system for measuring cell adhesion includes a gas impermeable housing including at least one microchannel defining at least one cell adhesion region, the at least one cell adhesion region being provided with at least one capturing agent that adheres a cell of interest to a surface of the at least one microchannel when a fluid sample containing cells is passed through the at least one microchannel, and an imaging system for measuring the adherence of cells of interest adhered by the at least one capturing agent to the surface of the at least one microchannel when the fluid sample is passed therethrough.

### RELATED APPLICATIONS

This application claims the benefit of U.S. Provisional Appln. Ser. No. 62/989,360, filed Mar. 13, 2020, U.S. Provisional Appln. Ser. No. 62/928,109, filed Oct. 30, 2019, U.S. Provisional Appln. Ser. No. 63/037,287, filed Jun. 10, 2020, U.S. Provisional Appln. Ser. No. 63/043,536, filed Jun. 24, 2020, U.S. Provisional Appln. Ser. No. 63/049,443, filed Jul. 8, 2020, and U.S. Provisional Appln. Ser. No. 63/072,502, filed Aug. 31, 2020, the subject matter of which are incorporated by reference herein their entirety.

### GOVERNMENT FUNDING

This invention was made with government support under Grant Nos. R01 HL133574, 1552782, OT2HL152643, 1706295, awarded by The National Institutes of Health. The United States government has certain rights to the invention.

### FIELD OF THE INVENTION

This application is related to biochips, and particularly relates to biochips having at least one microchannel provided with an agent for adhering and/or capturing cells of interest within a fluid sample delivered to the microchannel in order to perform cytological analysis.

### BACKGROUND

About 3 million people worldwide suffer from sickle cell disease (SCD), mostly in Africa, India, and the Middle East, with an estimated 100,000 affected in the U.S., according to the Centers for Disease Control, and Prevention. SCD affects 1 in 375 African American newborns born in the U.S.

The World Health Organization (WHO) has declared SCD a public health priority. The greatest burden of SCD is in low-income countries, especially in Africa. An estimated 50-80% of the babies born with SCD in Africa die before the age of 5, i.e., more than 600 babies die every day, due to lack of diagnosis. Very few infants are screened in Africa because of the high cost and level of skill needed to run traditional tests. Current methods are too costly and take too much time—2-6 weeks—to enable equitable and timely diagnosis. It is estimated by the WHO that 70% of SCD-related deaths are preventable with simple, cost-efficient interventions, such as early point-of-care (POC) diagnosis by newborn screening, followed by treatment and care. Early diagnosis through newborn screening, followed by simple interventions, has dramatically reduced the SCD-related mortality in the US. These strategies, however, have not been widely available in Africa and other third world countries due to limited resources.

Moreover, the initiation of vaso-occlusive crisis (VOC) events in SCD is a multicellular paradigm, likely triggered by aberrant adhesive interactions between red blood cells (RBCs) and microvascular bed, and further mediated by impaired RBC biophysical properties. In most cases, these events are followed by or simultaneous with the activation of other microcirculatory components, including white blood cells (WBCs), platelets, and endothelial cells. The interplay between these components, including the collective adhesive events, takes place under a wide spectrum of shear rates determined by the unique geometrical and morphological features of the human microvasculature, as well as by the local changes in the vascular dimensions upon cell-endothelium interactions. The flow conditions may dynamically and continuously change even within the same branch of the microvessel during this entire process.

### SUMMARY

This application describes a microfluidic system for measuring cell adhesion, detecting disorders associated with cell adhesion and/or measuring efficacy of or identifying agents capable of modulating cell adhesion. The microfluidic system can include a microfluidic device in the form of a biochip having microchannels provided or functionalized with a capturing agent for adhering and/or capturing cells of interest to be analyzed from a fluid sample, such as a blood sample obtained from a subject. In one example, the microfluidic device includes a housing formed of gas impermeable material that includes at least one microchannel that has at least one cell adhesion region. The at least one cell adhesion region includes at least one capturing or adhering agent that capture or adheres to cells of interest in a fluid sample when the fluid sample containing the cells is passed through the at least one microchannel. The microfluidic system can also include an imaging system for measuring the deformability, morphology, and/or quantity of the cells of interest adhered by the at least one capturing agent to the at least one microchannel when the fluid sample is passed therethrough. Optionally, the imaging system can measure the viscosity of the fluid sample.

When the fluid sample is blood, the cells of interest can be, for example, red blood cells (RBCs) or white blood cells (WBCs). In some embodiments, the capturing agents can include, for example, bioaffinity ligands, such as E-Selectin, P-Selectin, intracellular adhesion molecule 1 (ICAM-1) and vascular cellular adhesion molecule 1 (VCAM-1), that are functionalized to the surface of the microchannel and can be used to potentially adhere cells, such as WBCs and/or RBCs, in a fluid sample, such as blood. In other embodiments, the capturing agent can include, for example, cells, such as endothelial cells, including human umbilical vein endothelial cells and human pulmonary microvessel endothelial cells, which are functionalized to the surface of the microchannel and used to potentially adhere cells in a fluid sample, such as blood. In each case, the biochip is compact and requires a very small fluid sample from the subject, e.g., on the microscale.

The imaging system can detect and measure the deformability and/or morphology of cells in the fluid sample and/or quantity of adhered and/or captured cells perfused through the microchannels within at least one cell adhesion region of each microchannel The imaging system can optionally measure the viscosity of a fluid sample, such as blood, through the microchannel. The imaging system can be a lens-based imaging system or a lensless imaging system. The imaging system can include a processor to analyze the images of the microchannels and can provide real-time feedback to the subject of the results of the image acquisition/analysis. These results, in turn, can be readily transmitted to a primary care provider and/or stored in a medical record database.

In some embodiments, the microfluidic system can further include a reservoir fluidically connected with the one or more microfluidic channels, and a pump that perfuses fluid from the reservoir through the one or more microfluidic channels. The reservoir can contain cells, such as RBCs and WBCs, suspended in a fluid, such as blood or plasma.

In some embodiments, the cells can be RBCs, WBCs, stem cells, cancer cells, epithelial cells (e.g., epithelial cells of the cervix, pancreas, breast or bladder), B cells, T cells, or plasma cells. The cells, e.g., RBCs and WBCs, can be from a subject having or is suspected of having a disease (e.g., diabetes, infection with a virus, such as HIV, anemia, a hematological cancer, such as leukemia, a spleen disease, multiple myeloma, monoclonal gammopathy of undetermined significance, sickle cell disease, or spherocytosis).

In some embodiments, the imaging system can be configured to provide particle image velocimetry of fluid perfused through the microchannels. For example, the imaging system can be configured to take images of fluid as it passes through an imaging field of the microchannel These images can be sent to a control unit that includes a computer readable storage medium for storing the images and a processor that includes executable instructions for receiving sequential images, generating general velocity vector maps based on successive images, and generating mean flow velocity data from the velocity vector maps. The mean flow velocity data can be output from the processor to a display as raw data or as visual representation of the mean flow velocity. The mean flow velocity data or map can then be correlated to the viscosity of the fluid using the processor or another processor that outputs the viscosity data of the fluid as raw data or as visual depiction.

In some embodiments, the microchannels in the biochip can have a constant or variable width along their length. Varying the microchannel width provides continuously changing shear rates (shear gradient) along its length. Providing a shear gradient along the flow direction allows for the investigation of shear-dependent adhesion of cells at a single flow rate. The microchannel geometry can be configured such that both the mean flow velocity and shear stress decrease along the flow direction while the flow rate is constant.

The microfluidic system can simulate physiologically relevant shear gradients of microcirculatory blood flow at a constant single volumetric flow rate. Using this system, shear-dependent adhesion and deformability of cells, for example, RBCs and WBCs from subjects with disorders, such as SCD, can be investigated using capturing agents described herein. It was shown that shear dependent adhesion of cells, such as RBCs and WBCs, exhibit a heterogeneous behavior based on adhesion type and cell deformability in a microfluidic flow model, which correlates clinically with inflammatory markers and iron overload in patients with SCD. This revealed the complex dynamic interactions between RBC-mediated microcirculatory occlusion and clinical outcomes in SCD. These interactions may also be relevant to other microcirculatory disorders.

The microfluidic system can also include a micro-gas exchanger fluidly connected to the at least one microchannel for varying the oxygen content of the fluid sample containing the cells prior to perfusion through the at least one microchannel. The micro-gas exchanger can include a gas-permeable inner tube inserted within a gas-impermeable outer tube. Fluid, such as blood, containing the cells of interest can be delivered through the inner tube such that the fluid exchanges gases through the permeable tubing wall with a control gas, e.g., 5% CO₂ and 95% N₂, between the tubes. The oxygen content of the fluid exiting the micro-gas exchanger is controlled to thereby control the oxygen content of the fluid delivered to the microchannel.

In some embodiments, the micro-gas exchanger can be used to modulate the oxygen content of the fluid sample to a level associated with physiological normoxia, hypoxia, or hyperoxia.

In some embodiments, the microfluidic system can be used in methods for analyzing, characterizing and/or predicting cell, e.g., RBC and WBC, deformability, morphology, and/or adherence to various capturing agents, such as such as E-Selectin, P-Selectin, ICAM-1, VCAM-1, and/or endothelial cells, provided in the microchannels of the microfluidic device. In further embodiments, methods and devices are provided for diagnosing, assessing, characterizing, evaluating, and/or predicting disease based on cell deformability, morphology, and/or adherence to the capturing agents in the microchannels.

In some embodiment, the adherence of cells, such as RBCs and WBCs, to various capturing agents, such as E-Selectin, P-Selectin, ICAM-1, VCAM-1, and/or endothelial cells, provided in or functionalized to the microchannels of the microfluidic device can be used for evaluating, assessing, monitoring, and/or predicting disease status, disease prognosis, treatment course (e.g., therapeutic selection, dosing schedules, administration routes, etc.), response to treatment and/or treatment efficacy.

In some embodiments, the microfluidic device described herein can be used to assess the health of any of the subjects described herein, used to detect or determine the stage of any of the diseases or conditions described herein and can be used for determining the number of diseased versus healthy cells.

In other embodiments, a method for detecting a condition or disease in a subject can include obtaining cells, such as a RBCs, WBCs, stem cells, or plasma cells, from the subject and perfusing a fluid containing the cells through the microfluidic channel that includes various capturing agents, such as E-Selectin, P-Selectin, ICAM-1, VCAM-1, and/or endothelial cells, provided in or functionalized to the microchannels. The adherence of cells, such as RBCs and WBCs, to the various capturing agents provided in or functionalized to the microchannels of the microfluidic device can then be determined and compared to a standard or control to indicate whether the subject has the condition or disease; and optionally, diagnosing the subject as having the condition or disease based on the results. The appropriate standard or control can be the adherence of cells obtained from a subject who is identified as not having the condition or disease. The fluid viscosity in the microchannel can also be measured and compared to a control or standard to indicate or further characterize whether the subject has the condition or disease. In some embodiments, the perfusion of the fluid containing the cell can occur under normoxia or hypoxia conditions.

The condition or disease to be detected can be, for example, a hematological disorder, such as hematological cancer, anemia, infectious mononucleosis, HIV, malaria, leishmaniasis, sickle cell disease (SCD), babesiosis, spherocytosis, monoclonal gammopathy of undetermined significance or multiple myeloma.

In some embodiments, the microfluidic device can be used in a method of determining a subject having sickle cell disease risk of vaso-occlusive crises (VOC). The method can include obtaining blood or RBCs and/or WBCs, from the subject and perfusing a fluid containing the blood cells through the microfluidic channel that includes various capturing agents, such as E-Selectin, P-Selectin, ICAM-1, VCAM-1, and/or endothelial cells, provided in or functionalized to the microchannels. The adherence of cells, such as RBCs and WBCs, to the various capturing agents provided in or functionalized to the microchannels of the microfluidic device can then be determined and compared to a standard or control. The subject can have an increased risk of vaso-occlusive crises (VOC) when the measured adherence is greater than the control value.

Other embodiments described herein relate to a method for determining the effectiveness of a therapeutic agent for treating a condition or disease in a subject. The method can include obtaining cells, such as a RBCs, WBCs, stem cells, or plasma cells, from a subject suspected of having or a risk of a disorder, and perfusing a fluid containing the cells in the presence of the therapeutic agent through the microfluidic channel that includes various capturing agents, such as E-Selectin, P-Selectin, ICAM-1, VCAM-1, and/or endothelial cells, provided in the microchannels. The therapeutic agent can be administered to the cells prior to perfusing the fluid containing the cell through microchannel and/or to the microchannel and/or after adherence of the cells to the capturing agent. The adherence of the cells in the microchannel in the presence of the therapeutic agent can compared with control or standard to determine the effectiveness of the therapeutic agent. In some embodiments, the perfusion of the fluid containing the cell can occur under normoxia or hypoxia conditions.

Other embodiments relate to a method for identifying a candidate therapeutic agent for treating a condition or disease in a subject. The method can include obtaining cells, such as a RBCs, WBCs, stem cells, or plasma cells, from a subject suspected of having or a risk of a disorder, and perfusing a fluid containing the cells in the presence of the candidate therapeutic agent through the microfluidic channel that includes various capturing agents, such as E-Selectin, P-Selectin, ICAM-1, VCAM-1, and/or endothelial cells, provided in or functionalized to the microchannels. The therapeutic agent can be administered to the cells prior to perfusing the fluid containing the cell through microchannel and/or to the microchannel The therapeutic agent can be administered to the cells prior to perfusing the fluid containing the cell through microchannel and/or to the microchannel and/or after adherence of the cells to the capturing agent. The adherence of the cells in the microchannel in the presence of the therapeutic agent can compared with control or standard to determine whether the candidate therapeutic agent is useful for treating the condition or disease in the subject. In some embodiments, the perfusion of the fluid containing the cell can occur under normoxia or hypoxia conditions.

In any of the methods described herein, the fluid can be perfused, for example, through one or more microfluidic channels at a sheer stress that is indicative of physiological flow, e.g., about 0.5 dyne/cm² to about 2 dyne/cm² or about 1 dyne/cm² or a predetermined pressure gradient, e.g., about 20 mBar. Alternatively or additionally, the fluid is perfused at a predetermined temperature, e.g., a physiologically relevant temperature.

In other embodiments, the fluid can contain more than one type of cell (e.g., a mixture of both healthy and diseased cells). In one example, it contains RBCs, WBCs, epithelial cells, or a mixture thereof. In another example, it contains cancer cells. In yet another example, the fluid (e.g., whole blood) contains T cells, B cells, platelets, reticulocytes, mature red blood cells, or a combination thereof.

In some embodiments, data on the measurement of the adherence cells can be used in combination with data on the velocity and/or viscosity of the fluid in the microchannel under normoxia or hypoxia. The data obtained can include a value for the velocity for one of the cells in the fluid or the average velocity for a population of cells, the distance traveled by one of the cells, the time for one of the cells to travel a certain distance, the average distance traveled by a population of the cells, or the average time for a population of the cells to travel a certain distance in the microchannel The data on velocity and/or viscosity can be developed from one or more simulations of flow of a fluid in combination with experimental data.

In other embodiments, a method can include obtaining data for morphology and/or adherence of cells in the fluid perfused through the microchannels that includes the capturing agent, and determining one or more predicted values of flow behavior. The one or more predicted values can be determined and that correlated to flow behavior of any of the fluids described herein or elsewhere in this application to the one or more properties.

### BRIEF DESCRIPTION OF THE FIGURES

FIG. 1 is a schematic illustration of microfluidic system in accordance with an embodiment described herein.

FIG. 2A-B illustrate an example microfluidic biochip that evaluates cellular, membrane and adhesive interactions.

FIG. 3A is a schematic view of the human microvasculature system, with characteristic shear rates determined by the vessel geometry and local flow conditions.

FIG. 3B illustrates an example biochip with a microchannel configured to provide a shear gradient at a single flow rate.

FIG. 4 illustrates another example microfluidic device for capturing cells from fluid samples with an imaging system.

FIGS. 5A-5C illustrate another example microfluidic device for measuring RBC adhesion under physiological flow and hypoxic conditions

FIGS. 6A-6B illustrate images and plots showing a microfluidic whole blood assay of leukocyte adhesion to P-selectin and the inhibitory effect Crizanlizumab pre-treatment. (A) An assembled SCD Biochip platform consisting of 3 parallel microchannels with blood flow is shown. Insets: High-resolution phase-contrast microscope images of adherent leukocytes and reduction of leukocyte adhesion under Crizanlizumab pre-treatment are shown, Scale bars represent a length of 50 μm. (B) Crizanlizumab pre-treatment significantly reduced leukocyte adhesion to P-selectin in a dose-dependent manner Horizontal lines between individual groups represent a statistically significant difference based on a paired t-test. Error bars represent the standard error of the mean.

FIGS. 7A-7B illustrate images and plots crizanlizumab post-treatment promotes the detachment of adherent leukocytes to P-selectin. (A) Shown are the flow rates of the two programmed synchronous pumps adopted in the detachment experiments over time. Two images were recorded at two different time points to compare the effect of Crizanlizumab post-treatment. (B) Crizanlizumab post-treatment led to the detachment of adherent leukocytes on P-selectin. Horizontal line between individual groups represents a statistically significant based on a paired t-test (p<0.05). Scale bars represent a length of 50 μm.

FIGS. 8A-8C illustrate images and plots showing the standardized microfluidic approach for assessments of leukocyte adhesion to E-selectin in physiological flow conditions under normoxia and hypoxia. (A) An assembled microfluidic device containing 3 identical E-selectin-functionalized microchannels is shown. (B) Representative phase-contrast images showing adherent leukocytes of a single HbAA or HbSS sample under normoxia or hypoxia. Scale bars represent a length of 50 μm. (C) Leukocyte adhesion to E-selectin was hypoxia-enhanced in both tested HbAA (p=0.012, N=5) and HbSS (p=0.022, N=11) samples. Further, under both normoxia and hypoxia, HbSS samples had significantly greater leukocyte adhesion to E-selectin compared to HbAA samples (p=0.007 and p=0.023).

FIGS. 9A-9D illustrates images and plots showing the microfluidic assay used to assess adhesion of sickle RBCs to immobilized ICAM-1 under physiological flow conditions. (A) Assembled microdevice containing 3 separate micro-channels, each functionalized with ICAM-1. Arrow indicates the flow direction. (B) Recombinant human ICAM-1 protein was immobilized on the microchannel surface via a cross-linker agent, GMBS, to ensure consistent protein coverage throughout the micro-channel, as well as to prevent protein dissociation from the surface at high shear rates. (C) Representative image obtained following an adhesion experiment (upper panel). Enlarged images of the green boxes are shown (lower panel). Adherent sickle RBC populations possessed distinct morphologies: (i) RBC with a characteristic biconcave morphology and elongated elliptic shape, (ii) RBC with a nearly absent bi-concave morphology and further impaired elliptic shape, (iii) RBC with no biconcave morphology and elongated elliptic shape, (iv) highly sickled RBC with no biconcave morphology. (D) The number of adherent RBCs was significantly greater in samples from subjects with HbSS HbS variant HbAA. The horizontal lines represent the means; “n” represents the number of blood samples tested. A total of 106 blood samples were tested from 55 subjects with HbSS SCD. The scale bars are 5 mm.

FIGS. 10A-10D illustrate plots showing adhesion of HbSS RBCs to immobilized ICAM-1 in vitro is associated with subject hematological parameters. (A) Subjects were categorized into 2 groups based on their LDH levels and ARCs; group 1 (●) had higher LDH and ARCs. The categorization was performed based on the k-means clustering method. The shaded green and blue regions indicate reference ranges (normal) for LDH and ARC levels, respectively. (B) Subjects in group 2 had significantly higher RBC adhesion levels compared with the patients in group 1 (mean, 3159 6 4758 vs 453 6 1159, respectively). (C) There is a positive correlation between adherent RBC numbers and WBC counts. The shaded blue region represents the normal WBC range in healthy adults. (D) Subjects with clinically high WBC counts (0.11 3 10⁹/L) have significantly greater RBC adhesion to ICAM-1 in vitro. The horizontal lines in panels B and D represent the mean; “n” represents the number of subjects tested. The P values were calculated using the Mann-Whitney nonparametric U test. PCC, Pearson's correlation coefficient.

FIGS. 11A-11B illustrate plots showing adhesion of HbSS RBCs to immobilized ICAM-1 in vitro correlates clinically with HbF levels. (A) There is an inverse relationship between RBC adhesion to ICAM-1 and HbF level. The P value was based on 1-way ANOVA. (B) Subjects with higher HbF levels (using a previously defined ameliorative cutoff of 8.6% 22) had significantly lower numbers of adherent RBCs compared with those with lower HbF levels. The horizontal lines represent the means; “n” represents the number of subjects. The Mann-Whitney nonparametric U test was used to calculate the P value in panel B. PCC, Pearson's correlation coefficient.

FIG. 12 illustrates a graph showing association of RBC adhesion to ICAM-1 with select clinical phenotype in HbSS SCD. Subjects with a history of intracardiac or intrapulmonary shunt have significantly higher RBC adhesion compared with those with no history of shunt. A history of nephropathy or ACS does not have a significant association with RBC adhesion levels. The P value was calculated using a 2-sample Student t test. ns, not significant.

FIG. 13A-13C illustrate a graph showing the adhesion of HbSS RBCs to immobilized ICAM-1 is mediated by fibrinogen and is inhibited by LMWH. (A) Mean percentages of RBCs adherent to immobilized ICAM-1 following the treatment of blood samples with anti-α₄β₁ or anti-LFA-1 antibodies or the treatment of microchannels with recombinant human β₂ protein in 5 experiments. With vehicle treatment, a mean of 100% of RBCs adhered to immobilized ICAM-1. No significant reduction in HbSS RBC adhesion to immobilized ICAM-1 was observed (P>0.05). (B) Treatment of microchannels with fibrinogen decreased HbSS RBC adhesion to ICAM-1 in a concentration-dependent manner Shown are mean percentages of adherent RBCs after treatment with 1, 5, 10, or 20 mg/mL fibrinogen (n=5). (C) Treatment of blood samples with LMWH significantly inhibited HbSS RBC adhesion to ICAM-1. Shown are mean percentages of adherent RBCs following pretreatment with 0.1, 1, or 5 mg/mL LMWH in 5 experiments. The P values were calculated using 1-way ANOVA with the Dunnett post hoc test. Error bars represent 6 SEM.

FIGS. 14A-14D illustrate plots showing rolling adhesion of HbSS RBCs onto immobilized ICAM-1 under flow conditions. The data shown are the mean percentages of adherent or rolling RBCs onto immobilized ICAM-1 and the mean rolling velocities under shear rates of 500, 1000, 2000, 3000, 4000, and 5000 S⁻¹. A mean of 100% of HbSS RBCs adhered to immobilized ICAM-1 (including rolling adhesion and firm adhesion). (A) The number of adherent RBCs to immobilized ICAM-1 decreased with increasing shear rates. Shown are mean percentages of adherent RBCs (n 5 5). (B) Two overlapped frames, taken 1 second apart, extracted from a video depicting cell rolling behavior. (C) The number of rolling RBCs increased with increasing shear rates (n 5 5). (D) The velocities of rolling RBCs on immobilized ICAM-1 increased with increasing shear rates (n 5 5). Error bars represent 6 SEM.

FIG. 15 illustrates a plot showing a proposed ICAM-1-mediated HbSS RBC adhesion mechanism in SCD. Results are consistent with a model of firm adhesion of HbSS RBCs to the vasculature in the postcapillary venules under low physiological shear, which may be mediated by initial rolling adhesion of RBCs in the capillary under high physiological shear, facilitated by ICAM-1. RBCs may form firm attachment with ICAM-1 near the low shear sites throughout the microvasculature, contributing to impaired local flow conditions, as illustrated by the dashed oval.

FIG. 16 illustrates a plot showing sickle RBC adhesion to VCAM-1 under physiological flow conditions. A total number 12 blood samples were obtained from 12 different subjects with HbSS SCD. Adherent RBCs were categorized as deformable RBCs or non-deformable RBCs based on morphology analysis.

FIGS. 17A-17J illustrate an image and plots showing endothelium on a chip microfluidic platform for assessment of red blood cell and white blood cell (leukocyte) adhesion to activated endothelial cells. Representative images of adherent sickle RBCs to heme activated endothelial cells are shown in the control group (A, B on HUVECs and HPMECs) and in the imatinib treated group (C, D on HUVECs and HPMECs). Arrows indicate RBCs adherent to endothelium. (E) Sickle RBC adhesion to heme-activated endothelial cells is significantly reduced by imatinib (5 μM) treatment, compared with control (vehicle, DMSO) treatment (N=13 subjects, mean adhesion of untreated vs. imatinib-treated sickle cells±SEM=383±57 vs. 171±30, P<0.001, paired t-test). Representative images of adherent leukocytes to control HPMECs (F), TNF-α-activated HPMECs (G), and anti-E-selectin-treated TNF-α-activated HPMECs (H). Scale bars represent a length of 5 0 μm. (I) Overall, leukocyte adhesion to control HPMECs was negligible but significantly increased with TNF-α activation in the HbAA (N=3) and HbSS (N=6) subjects. Further, HbSS subjects had significantly greater leukocyte adhesion to TNF-α-activated HPMECs compared to HbAA subjects. (J) Treatment of TNF-α-activated HPMECs with adhesion blocking anti-E-selectin antibody significantly inhibited leukocyte adhesion in the HbSS subjects (N=5). HbAA: healthy, and HbSS: homozygous SCD. (RBC, red blood cell; HUVEC, human umbilical vein endothelial cells; HPMEC, human pulmonary microvascular endothelial cells).

FIG. 18 is a schematic illustration of the integrated micro-PIV microfluidic system. The microfluidic device is mounted on the stage of an inverted microscope equipped with a CCD video camera. 500 μl of pre-processing free whole blood samples are loaded in a reservoir and perfused through the microchannel at 20 mBar using a Fluigent Flow-EZ pump with positive pressure. Frame sequences are taken throughout the experiment using the high-speed camera and analyzed with the PIVlab software in Matlab to generate the velocity vectors. The corresponding mean flow velocity is calculated within a rectangular region of interest in the center spanning 80% of the entire field of view.

FIGS. 19A-19D illustrate plots showing the quantification of whole blood viscosity (WBV) for normal and SCD samples. (A) A total number of 19 blood samples were tested using the microfluidic platform to acquire the mean flow velocities. The clinical WBVs of these samples were obtained via a standard piston type commercially available viscometer. There was a logarithmic correlation between the mean flow velocity and measured WBV. (B) The whole blood samples were centrifuged to isolate RBCs, which were then mixed with subject's own plasma at a ratio of 1:1 to obtain a sample with 50% HCT before the viscosity measurements using the microfluidic platform. The microfluidic whole blood viscosity values were obtained by measuring the mean flow velocity first and converting that value to the clinical WBV using the correlation function in (A). Samples from homozygous (HbSS) SCD individuals had significantly greater WBV than samples from normal (HbAA) subjects at the 50% HCT level. The images below the graph are representative snapshots of the flow field and demonstrate similar brightness levels, which are indicative of sample HCT. (C) Viscosity was determined using unprocessed whole blood samples that maintained their specific HCT levels during the course of the experiments. The images below the graph are representative snapshots of the flow field for indicated blood sample types. Brightness of the images correlates with sample HCT level. (D) Average RBC counts and HCT levels for the SCD subjects are shown. Subjects with HbSS SCD had lower HCT levels and RBC counts relative to HbSC SCD subjects. P-values in (B) and (C) were calculated using Kruskal-Wallis non-parametric test with the Dunn's multiple comparison analysis. P-values in (D) were based on Mann-Whitney non-parametric test. N represents the number of subjects. Error bars represent standard deviation (SD).

FIGS. 20A-20C illustrate illustrates plots showing association of microfluidic WBV with hematological parameters. WBV moderately and positively correlates with subject HCT level (A), RBC count (B), and total hemoglobin level (C). PCC: Pearson correlation coefficient, and the p-value were based on a linear regression analysis. Blood samples from homozygous (HbSS) SCD subjects were used in this analysis.

FIGS. 21A-21B illustrate graphs showing the effect of transfusion (Tx) therapy on microfluidic WBV. (A) Subjects with a recent transfusion record (<3 months) have higher WBV compared to those who were not on transfusion. (B) HbS and HbA levels of subjects significantly vary based on Tx record. Subjects with a recent Tx have higher HbA and lower HbS levels compared to subjects with no recent Tx. The p-values were calculated based on the Student's t-test. The error bars represent standard deviation.

FIGS. 22A-22B illustrate graphs showing the microfluidic WBV correlates with RBC adhesion to LN in vitro. (A) Whole blood samples from HbSS SCD subjects (N=29) were first tested for WBV in our microfluidic platform. Adhesion of RBCs from the same samples to immobilized LN was quantified in separate microfluidic channels under physiologic flow conditions (shear stress=1 dyne/cm²). There was an inverse correlation between microfluidic WBV and number of adherent RBCs to LN. Higher WBV and lower WBV levels, which were measured via our microfluidic system, were chosen based on a threshold WBV value of 4 cP (dashed line), which was the lowest WBV of the healthy study population (HbAA). (B) Subjects with a lower WBV (<4 cP) displayed greater RBC adhesion to LN compared to those with a higher WBV (>4 cP). The p-value in (B) was based on the Student's t-test. The error bars represent standard deviation.

FIGS. 23A-23B illustrate plots showing the effect of hypoxia on microfluidic WBV. (A) Viscosity of whole blood samples from control subjects (HbAA, N=3)) and subjects with HbSS SCD (N=10) were quantified under hypoxia in our microfluidic devices. Whole blood samples were first injected into the microchannels in normoxic conditions, and the measurements were taken for 30 seconds. Then, the samples were exposed to hypoxia within the gas permeable inlet tubing so that they were already hypoxic (SpO₂ of ˜83%) before flowing into the microchannel Image acquisition was carried out 5 minutes after hypoxia induction for hypoxic viscosity measurements. WBV of control samples did not significantly alter when exposed to hypoxia while SCD samples became more viscous under hypoxic conditions. (B) Hypoxic WBV inversely correlated with hypoxic RBC adhesion. For hypoxic adhesion experiments, the blood samples were exposed to the same hypoxic gas mixture (5% CO₂-95% N₂) before being injected into the microchannels that were functionalized with LN. PCC and p-value were based on a linear regression analysis. P-value in (A) was calculated using the paired t-test test. Error bars represent standard deviation.

FIGS. 24A-24B illustrate an image and graph showing grayscale intensity values of the recorded videos over the time course of an entire normoxic WBV experiment in the microchannels. (A) Shown are representative images extracted from a typical video recorded for microfluidic whole blood viscosity measurement of a HbSS sample at specific time points for a duration of 50 seconds. Scale bars indicate 0.55 mm (B) Negligible changes in the gray value of the recorded videos were found over the 50-s time course. Error bars represent standard deviation. 4 HbSS samples were analyzed.

FIG. 25 illustrates a plot showing a comparison between theoretical and experimentally obtained mean flow velocities. To obtain the theoretical mean velocity, the microfluidic platforms were connected to a syringe pump filled with control blood samples (HbAA). The flow rate was varied between 2 μl/min and 10 μl/min, and the mean velocity was computed via the micro PIV setup. The theoretical mean velocity for each corresponding flow rate was found by dividing the flow rate by the cross-sectional area of the microchannel The experiments were repeated 3 times (N=3). Error bars represent standard deviation.

FIGS. 26A-26B illustrate an image and graphs showing a comparison of grayscale intensity values of the recorded videos between HbAA, HbSC, and HbSS samples. (A) Shown are representative images extracted from the middle of typical images recorded for microfluidic whole blood viscosity measurements of HbAA, HbSC and HbSS samples. Scale bars indicate 0.55 mm (B) The grayscale intensity values of images recorded for HbSS samples were significantly smaller compared to HbAA samples and HbSC samples (p<0.001&p=0.001, one-way ANOVA). Error bars represent standard deviation. N=4 in each group.

FIG. 27A-27B illustrate plots showing association of clinical WBV with hematological parameters. Clinical WBV moderately and positively correlates with subject HCT level (A) as well as RBC count (B). PCC: Pearson correlation coefficient, and the p-values were based on a linear regression analysis.

FIG. 28A-28B illustrate plots showing RBC adhesion to LN positively correlates with hemolytic biomarkers. (A) RBC adhesion is higher at increasing LDH levels. (B) A moderate positive correlation between RBC adhesion and absolute reticulocyte count exists. P-values are based on one-way ANOVA. The dashed lines represent a linear regression analysis.

FIG. 29A-29B illustrate plots showing grayscale intensity values of the recorded images over the time course of an entire hypoxia WBV measurement experiment. (A) Negligible changes in the grayscale intensity values of the recorded images were found over the 5-minute time course. Error bars represent standard deviation. 4 HbSS samples were analyzed. (B) Negligible changes in the grayscale intensity values of the recorded images were observed at the time point of 0 and 5 min. Error bars represent standard deviation. 7 HbSS samples were analyzed.

Other objects and advantages and a fuller understanding of the invention will be had from the following detailed description and the accompanying drawings.

### DETAILED DESCRIPTION

To facilitate the understanding of this invention, a number of terms are defined below. Terms defined herein have meanings as commonly understood by a person of ordinary skill in the areas relevant to the present invention. Terms such as “a”, “an”, and “the” are not intended to refer to only a singular entity but also plural entities and also includes the general class of which a specific example may be used for illustration. The terminology herein is used to describe specific aspects of the invention, but their usage does not delimit the invention, except as outlined in the claims.

The term “microchannels” as used herein refer to pathways through a medium, e.g., silicon, that allow for movement of liquids and gasses. Microchannels can therefore connect other components, i.e., keep components “in liquid communication.” While it is not intended that the present application be limited by precise dimensions of the channels, illustrative ranges for channels are as follows: the channels can be between 0.35 and 100 μm in depth (e.g., 50 μm) and between 50 and 10,000 μm in width (e.g., 400 μm). The channel length can be between 1 mm and 100 mm (e.g., about 27 mm).

The term “microfabricated”, “micromachined”, and/or “micromanufactured” as used herein means to build, construct, assemble or create a device on a small scale, e.g., where components have micron size dimensions or microscale.

The term “polymer” as used herein refers to a substance formed from two or more molecules of the same substance. Polymers may also be linear polymers in which the molecules align predominately in chains parallel or nearly parallel to each other. In a non-linear polymer, the parallel alignment of molecules is not required.

The term “lensless image” or “lensless mobile imaging system” as used herein refers to an optical configuration that collects an image based upon electronic signals as opposed to light waves. For example, a lensless image may be formed by excitation of a charged coupled device (CCD) sensor by emissions from a light emitting diode.

The term “charge-coupled device (CCD)” as used herein refers to a device for the movement of electrical charge, usually from within the device to an area where the charge can be manipulated, for example, a conversion into a digital value. A CCD provides digital imaging when using a CCD image sensor where pixels are represented by p-doped MOS capacitors.

The term “symptom” as used herein refers to any subjective or objective evidence of disease or physical disturbance observed by the patient. For example, subjective evidence is usually based upon patient self-reporting and may include, but is not limited to, pain, headache, visual disturbances, nausea, and/or vomiting. Alternatively, objective evidence is usually a result of medical testing including, but not limited to, body temperature, complete blood count, lipid panels, thyroid panels, blood pressure, heart rate, electrocardiogram, tissue, and/or body imaging scans.

The term “disease” or “medical condition”, as used herein, refers to any impairment of the normal state of the living animal that interrupts or modifies the performance of the vital functions. Typically manifested by distinguishing signs and symptoms, it is usually a response to: i) environmental factors (as malnutrition, industrial hazards or climate); ii) specific infective agents (as worms, bacteria or viruses); iii) inherent defects of the organism (as genetic anomalies); and/or iv) combinations of these factors.

The term “patient” or “subject” as used herein is a human or animal and need not be hospitalized. For example, out-patients, persons in nursing homes are “patients.” A patient may comprise any age of a human or non-human animal and therefore includes both adult and juveniles, i.e., children. It is not intended that the term “patient” connote a need for medical treatment and, thus, a patient may voluntarily or involuntarily be part of experimentation whether clinical or in support of basic science studies.

The term “derived from” as used herein refers to the source of a compound or sample. In one respect, a compound or sample may be derived from an organism or particular species.

The term “functionalized” or “chemically functionalized” as used herein means the addition of functional groups onto the surface of a material by chemical reaction(s). As will be readily appreciated by a person skilled in the art, functionalization can be employed for surface modification of materials in order to achieve desired surface properties, such as biocompatibility, wettability, and so on. Similarly, the term “biofunctionalization,” “biofunctionalized,” or the like, as used herein, means modification of the surface of a material to have desired biological function, which will he readily appreciated by a person of skill in the related art, such as bioengineering.

The term “sample” as used herein is used in its broadest sense and includes environmental and biological samples. Environmental samples include material from the environment such as soil and water. Biological samples may be animal, including, human, fluid, e.g., blood, plasma, and serum; solid, e.g., stool; tissue; liquid foods, e.g., milk; and solid foods, e.g., vegetables. A biological sample may comprise a cell, tissue extract, body fluid, chromosomes or extrachromosomal elements isolated from a cell, genomic DNA (in solution or bound to a solid support such as for Southern blot analysis), RNA (in solution or bound to a solid support such as for Northern blot analysis), cDNA (in solution or bound to a solid support) and the like.

The terms “capturing agent”, “bioaffinity ligand”, “binding component”, “molecule of interest”, “agent of interest”, “ligand” or “receptor” as used herein may be any of a large number of different molecules, biological cells or aggregates, and the terms are used interchangeably. Each capturing agent may be immobilized on a solid substrate and binds to an analyte being detected. Proteins, polypeptides, peptides, nucleic acids (nucleotides, oligonucleotides and polynucleotides), antibodies, ligands, saccharides, polysaccharides, microorganisms such as bacteria, fungi, and viruses, receptors, antibiotics, test compounds (particularly those produced by combinatorial chemistry), plant and animal cells organdies or fractions of each and other biological entities may each be a capturing agent. Each, in turn, also may be considered as analytes if same bind to a capturing agent on a microfluidic biochip.

The terms “bind” or “adhere” as used herein include any physical attachment or close association, which may be permanent or temporary. Generally, an interaction of hydrogen bonding, hydrophobic forces, van der Waals forces, covalent and ionic bonding etc., facilitates physical attachment between the molecule of interest and the analyte being measuring. The “binding” interaction may be brief as in the situation where binding causes a chemical reaction to occur. That is typical when the binding component is an enzyme and the analyte is a substrate for the enzyme. Reactions resulting from contact between the binding agent and the analyte are also within the definition of binding for the purposes of this application.

The term, “substrate” as used herein refers to surfaces as well as solid phases which may include a microchannel In some cases, the substrate is solid and may comprise PDMS. A substrate may also include components including, but not limited to, glass, silicon, quartz, plastic or any other composition capable of supporting photolithography.

The term, “photolithography”, “optical lithography” or “UV lithography” as used herein refers to a process used in microfabrication to pattern parts of a thin film or the bulk of a substrate. It uses light to transfer a geometric pattern from a photomask to a light-sensitive chemical “photoresist” or simply “resist,” on the substrate. A series of chemical treatments then either engraves the exposure pattern into or enables deposition of a new material in the desired pattern upon, the material underneath the photo resist. For example, in complex integrated circuits, a modern CMOS wafer will go through the photolithographic cycle up to 50 times.

Embodiments described herein relate to a microfluidic system for measuring cell adhesion, detecting disorders associated with cell adhesion and/or measuring efficacy of or identifying agents capable of modulating cell adhesion. The microfluidic system can include a microfluidic device or biochip and an analytic method for interrogation of cell adhesion to a surface, such as a microvasculature-mimicking surface at a single cell level, under physiological relevant shear stress (e.g., 0.5 dyne/cm² to about 2.0 dyne/cm²) and/or normoxia or hypoxia conditions. In some examples, the microfluidic device or system can quantify membrane, cellular, and adhesive properties of cells, such as red blood cells (RBCs) and white blood cells (WBCs) of a subject. This can be used, for example, to monitor disease severity, treatment response, treatment effectiveness in a clinically meaningful way.

The cells may be any mammalian cells. The cells may be any human cells. The cells, may be blood cells, such as RBCs and WBCs. The cells may be specific cells selected from the group consisting of lymphocytes, B cells, T cells, cytotoxic T cells, natural killer T cells, regulatory T cells, T helper cells, myeloid cells, granulocytes, basophil granulocytes, eosinophil granulocytes, neutrophil granulocytes, hypersegmented neutrophils, monocytes, macrophages, reticulocytes, platelets, mast cells, thrombocytes, megakaryocytes, dendritic cells, thyroid cells, thyroid epithelial cells, parafollicular cells, parathyroid cells, parathyroid chief cells, oxyphil cells, adrenal cells, chromaffin cells, pineal cells, pinealocytes, glial cells, glioblasts, astrocytes, oligodendrocytes, microglial cells, magnocellular neurosecretory cells, stellate cells, boettcher cells; pituitary cells, gonadotropes, corticotropes, thyrotropes, somatotrope, lactotrophs, pneumocyte, type I pneumocytes, type II pneumocytes, Clara cells; goblet cells, alveolar macrophages, myocardiocytes, pericytes, gastric cells, gastric chief cells, parietal cells, goblet cells, paneth cells, G cells, D cells, ECL cells, I cells, K cells, S cells, enteroendocrine cells, enterochromaffin cells, APUD cell, liver cells, hepatocytes, Kupffer cells, bone cells, osteoblasts, osteocytes, osteoclast, odontoblasts, cementoblasts, ameloblasts, cartilage cells, chondroblasts, chondrocytes, skin cells, hair cells, trichocytes, keratinocytes, melanocytes, nevus cells, muscle cells, myocytes, myoblasts, myotubes, adipocyte, fibroblasts, tendon cells, podocytes, juxtaglomerular cells, intraglomerular mesangial cells, extraglomerular mesangial cells, kidney cells, kidney cells, macula densa cells, spermatozoa, sertoli cells, leydig cells, oocytes, and mixtures thereof.

The cells may also be isolated from a healthy tissue or a diseased tissue, e.g., a cancer. Accordingly, the cells may be cancer cells. For example, the cells may be isolated or derived from any of the following types of cancers: breast cancer; biliary tract cancer; bladder cancer; brain cancer including glioblastomas and medulloblastomas; cervical cancer; choriocarcinoma; colon cancer; endometrial cancer; esophageal cancer; gastric cancer; hematological neoplasms including acute lymphocytic and myelogenous leukemia, e.g., B Cell CLL; T-cell acute lymphoblastic leukemia/lymphoma; hairy cell leukemia; chronic myelogenous leukemia, multiple myeloma; AIDS-associated leukemias and adult T-cell leukemia/lymphoma; intraepithelial neoplasms including Bowen's disease and Paget's disease; liver cancer; lung cancer; lymphomas including Hodgkin's disease and lymphocytic lymphomas; neuroblastomas; oral cancer including squamous cell carcinoma; ovarian cancer including those arising from epithelial cells, stromal cells, germ cells and mesenchymal cells; pancreatic cancer; prostate cancer; rectal cancer; sarcomas including leiomyosarcoma, rhabdomyosarcoma, liposarcoma, fibrosarcoma, and osteosarcoma; skin cancer including melanoma, Merkel cell carcinoma, Kaposi's sarcoma, basal cell carcinoma, and squamous cell cancer; testicular cancer including germinal tumors such as seminoma, non-seminoma (teratomas, choriocarcinomas), stromal tumors, and germ cell tumors; thyroid cancer including thyroid adenocarcinoma and medullar carcinoma; and renal cancer including adenocarcinoma and Wilms tumor. Cancer cells may be cells derived from any stage of cancer progression including, for example, precancerous cells, cancerous cells, and metastatic cells. Cancer cells also include cells from a primary tumor, secondary tumor or metastasis.

In other embodiments, the cells may be selected from the group consisting of cord-blood cells, stem cells, embryonic stem cells, adult stem cells, cancer stem cells, progenitor cells, autologous cells, isograft cells, allograft cells, xenograft cells, and genetically engineered cells. The cells may be induced progenitor cells. The cells may be cells isolated from a subject, e.g., a donor subject, which have been transfected with a stem cell associated gene to induce pluripotency in the cells. The cells may be cells which have been isolated from a subject, transfected with a stem cell associated gene to induce pluripotency, and differentiated along a predetermined cell lineage.

In one example, the cells are derived from whole blood of a subject, and the microfluidic system can be used to identify and/or measure the efficacy of therapeutic agents in treating various disorders by measuring adhesive properties of the cells under physiological flow or physiological relevant shear stress conditions and normoxia or hypoxia conditions.

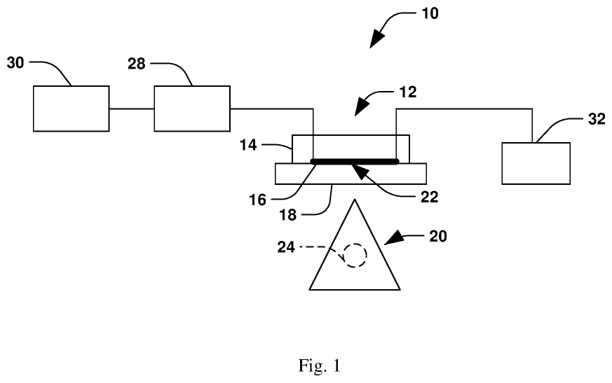

FIG. 1 illustrates a schematic view of a microfluidic system 10 in accordance with an embodiment described herein. The microfluidic system 10 includes a microfluidic device or biochip 12 that has a gas impermeable housing 14 and at least one microchannel 16 in the housing 14 that permits fluid sample flow through the housing 14. At least one of the microchannels 16 includes at least one cell adhesion region 22 with the microchannel 16. The fluidics associated the microchannels 16 can be arranged such that flow through each microchannel(s) travels in the same direction, or in opposite directions. When a microfluidic device 10 contains at least two microchannels and the fluidics associated the channels are arranged such that flow through each microchannel(s) travels in the same direction, the microchannels are typically either partially fluidically isolated or fluidically isolated. When a microfluidic device 10 contains at least two microchannels and the fluidics associated the channels are arranged such that flow through each channel(s) travels in opposite directions, the microchannels are typically fluidically isolated. Microchannels that are “fluidically isolated” are configured and designed such that there is no fluid exchanged directly between the microchannels. Microchannels that are “partially fluidically isolated” are configured and designed such that there is partial (e.g., incidental) fluid exchanged directly between the channels.

The housing 14 including the at least one microchannel 16 can further contain a substantially planar transparent wall 18 that defines a surface of at least one of the microchannels 16. This substantially planar transparent wall 18, which can be, for example, glass or plastic, permits observation into the microfluidic channel 16 by an imaging system 20 (e.g., microscopy) so that at least one measurement of each cell that passes through the cell adhesion region 22 of one of the microfluidic channels 16 can be obtained. In one example, the transparent wall has a thickness of 0.05 mm to 1 mm. In some cases, the transparent wall 18 may be a microscope cover slip, or similar component. Microscope coverslips are widely available in several standard thicknesses that are identified by numbers, as follows: No. 0-0.085 to 0.13 mm thick, No. 1-0.13 to 0.16 mm thick, No. 1.5-0.16 to 0.19 mm thick, No. 2-0.19 to 0.23 mm thick, No. 3-0.25 to 0.35 mm thick, No. 4-0.43 to 0.64 mm thick, any one of which may be used as a transparent wall 18, depending on the device, microscope, and detection strategy.

In some embodiments, the microfluidic channel(s) 16 may have a depth or height in a range of 0.5 μm to 100 μm, 0.1 μm to 100 μm, 1 μm to 50 μm, 1 μm to 50 μm, 10 μm to 40 μm, 5 μm to 15 μm, 0.1 μm to 5 μm, or 2 μm to 5 μm. The microfluidic channel(s) may have a depth or height of up to 0.5 μm, 1 μm, 1.5 μm, 2.0 μm, 2.5 μm, 3.0 μm, 3.5 μm, 4.0 μm, 4.5 μm, 5.0 μm, 5.5 μm, 6.0 μm, 6.5 μm, 7.0 μm, 7.5 μm, 8.0 μm, 8.5 μm, 9.0 μm, 9.5 μm, 10 μm, 20 μm, 30 μm, 40 μm, 50 μm, 75 μm, 100 μm, or more.

In some embodiments, the at least one microchannel 16 can have a cross-sectional area, perpendicular to the flow direction, of 1 μm², 10 μm², 20 μm², 30 μm², 40, μm², 50 μm², 60 μm², 70 μm², 80 μm², 90 μm², 100 μm², 150 μm², 200 μm², 300 μm², 400 μm², 500 μm², 600 μm², 700 μm², 800 μm², 900 μm², 1000 μm², or more.

The microfluidic device 10 may be designed and configured to have a channel cross-sectional area, perpendicular to the flow direction, in a range of 1 μm² to 10 μm², 10 μm² to 50 μm², 50 μm² to 100 μm², 100 μm² to 500 μm², 500 μm² to 1500 μm², for example.

The microfluidic device 10 may be designed and configured to produce any of a variety of different shear rates (e.g., up to 100 dynes/cm²). For example, the microfluidic device 10 may be designed and configured to produce a shear rate in a range of 0.1 dynes/cm² to 10 dynes/cm², 0.5 dynes/cm² to 5 dynes/cm², 0.5 dynes/cm² to 2 dynes/cm², 0.6 dynes/cm² to 1.5 dynes/cm², 0.7 dynes/cm² to 1.3 dynes/cm², 0.8 dynes/cm² to 1.2 dynes/cm², or 0.9 dynes/cm² to 1.1 dynes/cm², or 1 dynes/cm².

Each microchannel 16 can have a constant width or a width that continuously changes in a direction of the fluid sample flow through the microchannel. Varying the microchannel width provides continuously changing shear rates (shear gradient) along its length. Providing a shear gradient along the flow direction allows for the investigation of shear-dependent adhesion of cells at a single flow rate. The microchannel geometry can be configured such that both the mean flow velocity and shear stress decrease along the flow direction while the flow rate is constant.

In some embodiments, the microfluidic system 10 can simulate physiologically relevant shear gradients (e.g., 0.5 dynes/cm² to about 2 dynes/cm²) of microcirculatory blood flow at a constant single volumetric flow rate. Using this system, shear-dependent adhesion and deformability of cells, for example, RBCs and WBCs from subjects with disorders, such as SCD can be investigated using capturing agents described herein. It was shown that shear dependent adhesion of cells, such as RBCs and WBCs, exhibit a heterogeneous behavior based on adhesion type and cell deformability in a microfluidic flow model, which correlates clinically with inflammatory markers and iron overload in patients with SCD. This revealed the complex dynamic interactions between RBC-mediated microcirculatory occlusion and clinical outcomes in SCD. These interactions may also be relevant to other microcirculatory disorders.

In some embodiments, the cell adhesion region 22 of the microchannel 16 is functionalized with at least one capturing agent that captures or adheres a cell of interest to a surface of the microchannel when a sample fluid containing cells is passed or perfused through the at least one microchannel. If the housing includes multiple microchannels, each microchannel can be functionalized with a different capturing agent to adhere different cells of interest thereto. In any case, each microchannel is configured to receive and provide cell adhesion analysis of a microvolume fluid sample.

In some embodiments. the capturing agents can include, for example, bioaffinity ligands or adhesion molecules that are associated with an activated phenotype in SCD. Such bioaffinity ligands or adhesion molecules can include, for example, E-Selectin, P-Selectin, intracellular adhesion molecule 1 (ICAM-1) and vascular cellular adhesion molecule 1 (VCAM-1). E-selectin and P-selectin are expressed on the surfaces of endothelial cells in response to inflammatory stimuli and mediates leukocyte rolling and adhesion on endothelial cells. ICAM-1 and VCAM-1 are also expressed on the surface of endothelial cells in response to inflammatory stimuli and helps regulate inflammation associated cellular adhesion and transmigration of WBCs. VCAM-1 further mediates adhesion of sickle RBCs, particularly reticulocytes, which are young RBCs. E-Selectin, P-Selectin, ICAM-1, and/or VCAM-1 can adhere to cells, such as WBCs and/or RBCs, and be used to detect and/or measure WBC and/or RBC adherence under physiological relevant shear stress and normoxic and hypoxic conditions.

In other embodiments, the capturing agent can include, for example, cells, such as endothelial cells, including human umbilical vein endothelial cells and human pulmonary microvessel endothelial cells, that can potentially adhere to cells, such as WBCs adhesion and/or RBCs under physiological relevant shear stress and normoxic and hypoxic conditions. The cells, e.g., endothelial cells, can be adhered or functionalized to the surface of the cell adhesion region of the microchannel and cultured under physiological relevant flow conditions. The cultured cells, such as cultured endothelial cell can be activated with a variety of stimuli including heme, TNF-α, hydrogen, peroxide, and thrombin to mimic various physiological or pathological conditions.

The microfluidic system also includes an imaging system for measuring the deformability, morphology, and/or quantity of the cells of interest in the cell adhesion region and/or adhered to the at least one capturing agent of at least one microchannel when the fluid sample is passed or perfused through the microchannel under, for example, physiological relevant shear stress and normoxia or hypoxia conditions.

The imaging system 20 can detect and measure through the at least one optically transparent wall the morphology and/or quantity of adhered and/or captured cells of interest within each microchannel and optionally the viscosity of the fluid sample. The imaging system 20 can be a lens-based imaging system, lensless imaging system, and/or mobile imaging system, e.g., cellular phone camera. The imaging system 20 can include a control unit 24, which can a include a computer readable storage unit and a processor to analyze the images of the microchannels and provide real-time feedback to a subject of the results of the image acquisition/analysis. These results, in turn, can be readily transmitted to a primary care provider and/or stored in a medical record database.

In some embodiments, the imaging system 20 can be configured to provide particle image velocimetry of fluid in the microchannels. For example, the imaging system 20 can be configured to take images of fluid as it passes through an imaging field of the microchannel These images can be sent to control unit that includes a computer readable storage medium for storing the images and a processor that include executable instructions for receiving sequential images, generating general velocity vector maps based on successive images, and generating mean flow velocity data from the velocity vector maps. The mean flow velocity data can be output from the processor to a display as raw data or as visual representation of the mean flow velocity. The mean flow velocity data or map can be correlated to viscosity of the fluid using the processor or another processor that outputs the viscosity date of the fluid as raw data or as visual depiction.

The image processing may be implemented using hardware, software or a combination thereof. When implemented in software, the software code can be executed on any suitable processor or collection of processors, whether provided in a single computer or distributed among multiple computers. Such processors may be implemented as integrated circuits, with one or more processors in an integrated circuit component. Though, a processor may be implemented using circuitry in any suitable format.

Further, it should be appreciated that a computer may be embodied in any of a number of forms, such as a rack-mounted computer, a desktop computer, a laptop computer, or a tablet computer. Additionally, a computer may be embedded in a device not generally regarded as a computer but with suitable processing capabilities, including a Personal Digital Assistant (PDA), a smart phone or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible format.

Such computers may be interconnected by one or more networks in any suitable form, including as a local area network or a wide area network, such as an enterprise network or the Internet. Such networks may be based on any suitable technology and may operate according to any suitable protocol and may include wireless networks, wired networks or fiber optic networks

Also, the various methods or processes outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

In this respect, a computer readable medium (or multiple computer readable media) (e.g., a computer memory, one or more floppy discs, compact discs (CD), optical discs, digital video disks (DVD), magnetic tapes, flash memories, circuit configurations in Field Programmable Gate Arrays or other semiconductor devices, or other non-transitory, tangible computer storage medium) can be encoded with one or more programs that, when executed on one or more computers or other processors, perform methods that implement the various embodiments described herein. The computer readable medium or media can be transportable, such that the program or programs stored thereon can be loaded onto one or more different computers or other processors to implement various aspects described herein. As used herein, the term “non-transitory computer-readable storage medium” encompasses only a computer-readable medium that can be considered to be a manufacture (i.e., article of manufacture) or a machine.

The terms “program” or “software” are used herein in a generic sense to refer to any type of computer code or set of computer-executable instructions that can be employed to program a computer or other processor to implement various aspects as discussed above. Additionally, it should be appreciated that according to one aspect of this embodiment, one or more computer programs that when executed perform methods of described herein need not reside on a single computer or processor, but may be distributed in a modular fashion amongst a number of different computers or processors to implement various aspects herein.

Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

In some embodiments, the microfluidic system 10 can further include a reservoir 28 fluidically connected with the one or more microfluidic channels 16, and a pump 30 that perfuses fluid from the reservoir 28 through the one or more microchannels 16 to a waste reservoir 32. The pump 30 can designed and configured to create a pressure to create a pressure (gauge pressure) in at least one of the microchannels 16 of up to 50 Pa, 100 Pa, 200 Pa, 300 Pa, 400 Pa, 500 Pa, 600 Pa, 700 Pa, 800 Pa, 900 Pa, 1 kPa, 2 kPa, 5 kPa, 10 kPa or more. The pump 30 may be designed and configured to create a pressure (gauge pressure) in the channel in a range of 50 Pa to 200 Pa, 100 Pa to 500 Pa, 100 Pa to 800 Pa, 100 Pa to 1 kPa, 500 Pa to 5 kPa, or 500 Pa to 10 kPa.

The microfluidic system 10 may also be designed and configured to create an average fluid velocity within the channel of up to 1 μm/s, 2 μm/s, 5 μm/s, 10 μm/s, 20 μm/s, 50 μm/s, 100 μm/s, or more.

The microfluidic system 10 may be designed and configured to create an average fluid velocity within at least one microchannel 16 in a range of 1 μm/s to 5 μm/s, 1 μm/s to 10 μm/s, 1 μm/s to 20 μm/s, 1 μm/s to 50 μm/s, 10 μm/s to 100 μm/s, or 10 μm/s to 200 μm/s, for example.

In some embodiments, the reservoir 28 contains cells, such as RBCs and WBCs, suspended in a fluid, such as blood or plasma.

In some embodiments, the cells can be RBCs, WBCs, stem cells, cancer cells, epithelial cells (e.g., epithelial cells of the cervix, pancreas, breast or bladder), B cells, T cells, or plasma cells that can be obtained from a subject having or is suspected of having a disease (e.g., diabetes, infection with a virus such as HIV, anemia, a hematological cancer, such as leukemia, a spleen disease, multiple myeloma, monoclonal gammopathy of undetermined significance, sickle cell disease, or spherocytosis).

In some embodiments, the microfluidic system 10 can further includes a micro-gas exchanger (not shown) fluidly connected to the at least one microchannel 16 for varying the oxygen content of the fluid sample containing the cells. The micro-gas exchanger can include a gas-permeable inner tube inserted within a gas-impermeable outer tube. Fluid, such as blood or synovial fluid, containing the cells of interest can be delivered through the inner tube such that the fluid exchanges gases through the permeable tubing wall with a control gas, e.g., 5% CO₂ and 95% N₂, between the tubes. The oxygen content of the fluid exiting the micro-gas exchanger is controlled to thereby control the oxygen content of the fluid delivered to the microchannel.

By way of example, the micro-gas exchanger can include concentric inner and outer tubes. The inner tube has a gas-permeable wall defining a central passage extending the entire length of the inner tube. The outer tube has a gas impermeable wall defining a central passage extending the entire length of the outer tube. An annular space is formed between the tubes. The central passage receives the fluid sample and is in fluid communication with one or more inlet ports of the microfluidic device. Each inlet port can be fluidly connected to the same micro-gas exchanger or a different micro-gas exchanger to specifically tailor the fluid delivered to each microchannel. An outlet tube is connected to each outlet port of the micro-gas exchanger. A controlled gas flow takes place in the annular space between the concentric tubes and fluid flows inside the inner tube. When the fluid sample is blood, deoxygenation of the sample occurs due to gas diffusion (5% CO₂ and 95% N₂) through the inner gas-permeable wall.

In some embodiments, the microfluidic system can be used in methods for analyzing, characterizing and/or predicting the adherence of cells, such as RBCs and WBCs, to various capturing agents, such as such as E-Selectin, P-Selectin, ICAM-1, VCAM-1, and/or endothelial cells, provided in the microchannels of the microfluidic device. In further embodiments, methods and devices are provided for diagnosing, assessing, characterizing, evaluating, and/or predicting disease based on the adherence of the cells to the capturing agents in microchannels as well as the viscosity of a fluid sample, such as blood.

Any appropriate condition or disease of a subject may be evaluated using the methods herein, typically provided that a cell may be obtained from the subject that has a material property (e.g., deformability, adherence, etc.) that is indicative of the condition or disease. The condition or disease to be detected may be, for example, a hematological disorder, such as hematological cancer, anemia, infectious mononucleosis, HIV, malaria, leishmaniasis, sickle cell disease (SCD), babesiosis, spherocytosis, monoclonal gammopathy of undetermined significance or multiple myeloma. Examples of hematological cancer include, but are not limited to, Hodgkin's disease, Non-Hodgkin's lymphoma, Burkitt's lymphoma, anaplastic large cell lymphoma, splenic marginal zone lymphoma, hepatosplenic T-cell lymphoma, angioimmunoblastic T-cell lymphoma (AILT), multiple myeloma, Waldenstrom macroglobulinemia, plasmacytoma, acute lymphocytic leukemia (ALL), chronic lymphocytic leukemia (CLL), B cell CLL, acute myelogenous leukemia (AML), chronic myelogenous leukemia (CML), T-cell prolymphocytic leukemia (T-PLL), B-cell prolymphocytic leukemia (B-PLL), chronic neutrophilic leukemia (CNL), hairy cell leukemia (HCL), T-cell large granular lymphocyte leukemia (T-LGL) and aggressive NK-cell leukemia. The foregoing diseases or conditions are not intended to be limiting. It should thus be appreciated that other appropriate diseases or conditions may be evaluated using the methods disclosed herein.

Methods are also provided for detecting and characterizing a leukocyte-mediated condition or disease. For example, methods are provided for detecting and characterizing a leukocyte-mediated condition or disease associated with the lungs of a subject being highly susceptible to injury, possibly due to activated leukocytes with altered deformability, having altered ability to circulate through the pulmonary capillary bed. Methods such as these, and others disclosed herein, can also be applied to detect and/or characterize septic shock (sepsis) that is associated with both rigid and activated neutrophils. Such neutrophils can, in some cases, occlude capillaries and damage organs where changes in neutrophil cytoskeleton are induced by molecular signals leading to decreased deformability.

Further, certain methods described herein provide for measurement of adhesive properties of a cell population, in combination with or separate from measurement of the deformability of the cell population. The combination of determining cytoadhesive properties and the deformative properties of a cell population, particularly a cell population containing a plurality of different cell types (e.g., RBCs and WBCs), may be used to generate a “Health Signature” that comprises an array of properties that can be tracked in a subject over a period of time. Such a Health Signature may facilitate effective monitoring of a subject's health over time. Such monitoring may lead to an early detection of potential acute or chronic infection, or other disease, disorder, fitness, or condition. In some cases, further, knowledge of the overall rheology of a material, along with either the deformative or cytoadhesive property of a cell, allows the determination of the other property.

In some embodiment, the adherence of cells, such as RBCs and WBCs, to various capturing agents, such as E-Selectin, P-Selectin, ICAM-1, VCAM-1, and/or endothelial cells, provided in the microchannels of the microfluidic device devices can be used for evaluating, assessing, monitoring, and/or predicting disease status, disease prognosis, treatment course (e.g., therapeutic selection, dosing schedules, administration routes, etc.), response to treatment and/or treatment efficacy.

In some embodiments, the microfluidic device described herein can be used to assess the health of any of the subjects described herein, used to detect or determine the stage of any of the diseases or conditions described herein and can be used for determining the number of diseased versus healthy cells.

In other embodiments, a method for detecting a condition or disease in a subject can include obtaining cells, such as a RBCs, WBCs, stem cells, or plasma cells, from the subject and perfusing a fluid containing the cells through the microfluidic channel that includes various capturing agents, such as E-Selectin, P-Selectin, ICAM-1, VCAM-1, and/or endothelial cells, provided in or functionalized to the microchannels.

The cells can be obtained directly or indirectly by acquiring a biological sample from a subject. For example, a biological sample may be obtained (e.g., at a point-of-care facility, e.g., a physician's office, a hospital, laboratory facility) by procuring a tissue or fluid sample (e.g., blood draw, marrow sample, spinal tap) from a subject. Alternatively, a biological sample may be obtained by receiving the biological sample (e.g., at a laboratory facility) from one or more persons who procured the sample directly from the subject. The biological sample may be, for example, a tissue (e.g., blood), cell (e.g., hematopoietic cell such as hematopoietic stem cell, leukocyte, or reticulocyte, stem cell, or plasma cell), vesicle, biomolecular aggregate or platelet from the subject.

The adherence of cells, such as RBCs and WBCs, to the various capturing agents provided in the microchannels of the microfluidic device can then be determined and compared to a standard or control to indicate whether the subject has the condition or disease; and optionally, diagnosing the subject as having the condition or disease based on the results. The appropriate standard or control can be the adherence of cells obtained from a subject who is identified as not having the condition or disease. The fluid viscosity in the microchannel can also be measured and compared to a control or standard to indicate or further characterize whether the subject has the condition or disease.

An “appropriate standard” is a parameter, value or level indicative of a known outcome, status or result (e.g., a known disease or condition status). An appropriate standard can be determined (e.g., determined in parallel with a test measurement) or can be pre-existing (e.g., a historical value, etc.). The parameter, value or level may be, for example, a flow or adherence characteristic (e.g., flow time), a value representative of a mechanical property, a value representative of a rheological property, etc. For example, an appropriate standard may be the flow or adherence characteristic of a cell obtained from a subject known to have a disease, or a subject identified as being disease-free. In the former case, a lack of a difference between the flow or adherence characteristic and the appropriate standard may be indicative of a subject having a disease or condition. Whereas in the latter case, the presence of a difference between the flow or adherence characteristic and the appropriate standard may be indicative of a subject having a disease or condition. The appropriate standard can be a mechanical property or rheological property of a cell obtained from a subject who is identified as not having the condition or disease or can be a mechanical property or rheological property of a cell obtained from a subject who is identified as having the condition or disease.

The magnitude of a difference between a parameter, level or value and an appropriate standard that is indicative of known outcome, status or result may vary. For example, a significant difference that indicates a known outcome, status or result may be detected when the level of a parameter, level or value is at least 1%, at least 5%, at least 10%, at least 25%, at least 50%, at least 100%, at least 250%, at least 500%, or at least 1000% higher, or lower, than the appropriate standard. Similarly, a significant difference may be detected when a parameter, level or value is at least 2-fold, at least 3-fold, at least 4-fold, at least 5-fold, at least 6-fold, at least 7-fold, at least 8-fold, at least 9-fold, at least 10-fold, at least 20-fold, at least 30-fold, at least 40-fold, at least 50-fold, at least 100-fold, or more higher, or lower, than the level of the appropriate standard. Significant differences may be identified by using an appropriate statistical test. Tests for statistical significance are well known in the art and are exemplified in Applied Statistics for Engineers and Scientists by Petruccelli, Chen and Nandram Reprint Ed. Prentice Hall (1999).

For example, it was found that increased RBC adhesion to ICAM-1 correlates with hemolysis and a history of right-to-left shunts. Moreover, variations in RBC adhesion to ICAM-1 over time appear to correlate with LDH and ARC: an increasing or decreasing level of LDH or ARC corresponds to an increase or decrease in RBC adhesion. These observations are consistent with the “hyperhemolytic paradigm” that establishes that hemolysis in SCD increases endothelial dysfunction along with increased free plasma hemoglobin and NO biodeficiency and consumption.

In some embodiments, the microfluidic device can be used in a method of determining a subject having sickle cell disease risk of vaso-occlusive crises (VOC). The method can include obtaining blood or RBCs and/or WBCs, from the subject and perfusing a fluid containing the blood cells through the microfluidic channel that includes various capturing agents, such as E-Selectin, P-Selectin, ICAM-1, VCAM-1, and/or endothelial cells, provided in or functionalized to the microchannels. The adherence of cells, such as RBCs and WBCs, to the various capturing agents provided in or functionalized to the microchannels of the microfluidic device can then be determined and compared to a standard or control. The subject can have an increased risk of vaso-occlusive crises (VOC) when the measured adherence is greater than the control value.

Other embodiments described herein relate to a method for identifying the effectiveness of a candidate therapeutic agent for treating a condition or disease in a subject. The method can include obtaining cells, such as a RBCs, WBCs, stem cells, or plasma cells, from a subject suspected of having or a risk of a disorder, and perfusing a fluid containing the cells in the presence of the therapeutic agent through the microfluidic channel that includes various capturing agents, such as E-Selectin, P-Selectin, ICAM-1, VCAM-1, and/or endothelial cells, provided in the microchannels. The candidate therapeutic agent can be administered the cells prior to perfusing the fluid containing the cell through microchannel and/or to the microchannel and/or after the cells had adhered to the capturing. The adherence of the cells in the microchannel in the presence of the therapeutic agent can compared with control or standard to determine the effectiveness of the therapeutic agent. In some embodiments, the perfusion of the fluid containing the cell can occur under normoxia or hypoxia conditions.

For example, leukocyte adhesion to P-selectin can be measured under physiologic flow conditions using the microfluidic system. As shown in the examples, the system can reveal the association between patient-specific adhesion profiles and clinical phenotypes. Specifically, data in the examples support the inhibitory effect of pre-emptive Crizanlizumab on P-selectin mediated leukocyte adhesion, and of post-treatment Crizanlizumab on leukocyte detachment.

Other embodiments described herein relate to a method for determining the effectiveness of a therapeutic agent for treating a condition or disease in a subject. The method can include obtaining cells, such as a RBCs, WBCs, stem cells, or plasma cells, from a subject suspected of having or a risk of a disorder, and perfusing a fluid containing the cells in the presence of the therapeutic agent through the microfluidic channel that includes various capturing agents, such as E-Selectin, P-Selectin, ICAM-1, VCAM-1, and/or endothelial cells, provided in the microchannels. The therapeutic agent can be administered to the cells prior to perfusing the fluid containing the cell through microchannel and/or to the microchannel and/or after adherence of the cells to the capturing agent. The adherence of the cells in the microchannel in the presence of the therapeutic agent can compared with control or standard to determine the effectiveness of the therapeutic agent. In some embodiments, the perfusion of the fluid containing the cell can occur under normoxia or hypoxia conditions.

In any of the methods described herein, the fluid can be perfused, for example, through one or more microfluidic channels at a sheer stress that is indicative of physiological flow, e.g., about 0.5 dyne/cm² to about 2 dyne/cm² or about 1 dyne/cm² or a predetermined pressure gradient, e.g., about 20 mBar. Alternatively or additionally, the fluid is perfused at a predetermined temperature, e.g., a physiologically relevant temperature.

In other embodiments, the fluid can contain more than one type of cell (e.g., a mixture of both healthy and diseased cells). In one example, it contains RBCs, WBCs, epithelial cells, or a mixture thereof. In another example, it contains cancer cells. In yet another example, the fluid (e.g., whole blood) contains T cells, B cells, platelets, reticulocytes, mature red blood cells, or a combination thereof.

In some embodiments, data on the measurement of the adherence of cells can be used in combination with data on the velocity and/or viscosity of the fluid in the microchannel under normoxia or hypoxia. The data obtained can include a value for the velocity for one of the cells in the fluid or the average velocity for a population of cells, the distance traveled by one of the cells, the time for one of the cells to travel a certain distance, the average distance traveled by a population of the cells, or the average time for a population of the cells to travel a certain distance in the microchannel The data on velocity and/or viscosity can be developed from one or more simulations of flow of a fluid in combination with experimental data.

In some embodiments, the microfluidic system describe herein can be utilized for simultaneous measurement of whole blood viscosity (WBV) and RBC adhesion for emerging targeted as well as curative therapies. For instance, WBV and RBC adhesion levels can be assessed before and after therapeutic interventions targeted at HCT augmentation, adhesion mitigation, and/or before and after a curative therapies, in order to assess changes in blood and RBCs with therapy.

In other embodiments, a method can including obtaining data for morphology and/or adherence of cells in the fluid perfused through the microchannels that include the capturing agent, and determining one or more predicted values of flow behavior. The one or more predicted values are determined and that correlates flow behavior of any of the fluids described herein or elsewhere in this application to the one or more properties.

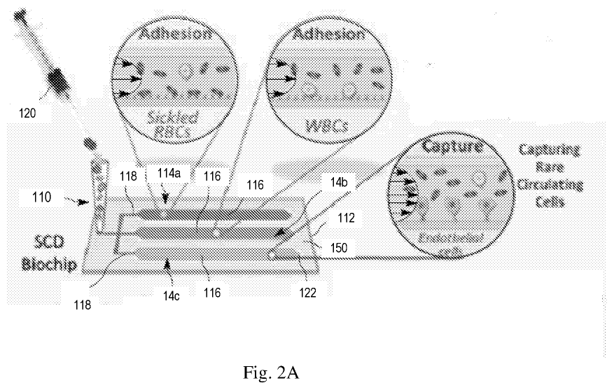

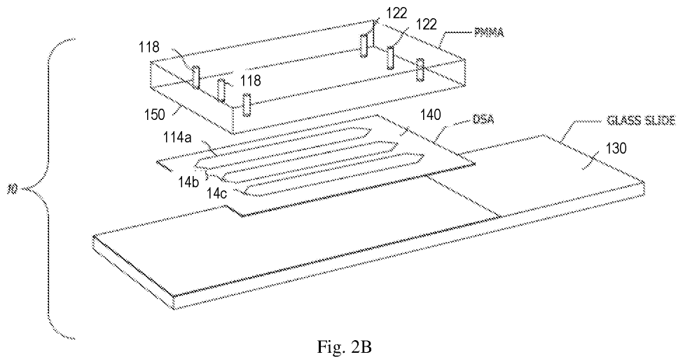

FIG. 2A illustrates an example microfluidic device 110 for measuring cell adhesion and interactions. The microfluidic device 110 includes a housing 112 defining at least one channel 114—here a plurality of channels 114a-114c—that each includes a cell adhesion or adherence region 16. Each channel 114a-114c is fluidly connected to an inlet port 118 at one end and an outlet port (not shown) at another end. Although FIG. 2A depicts three channels 114a-114c, the microfluidic device 110 can include more or fewer than three channels. The size of each channel 114a-114c should be large enough to prevent clogging of the channels when a fluid sample 120, e.g., blood fluid or a fluid containing cells to be analyzed, is passed through the channels.

Referring to FIG. 2B, the microfluidic biochip 110 can include a multilayer structure formed of a base layer 130, an intermediate layer 140, and a cover layer 150. The channels 114a-114c are formed in the intermediate layer 140. A first end of each channel 114a-114c is aligned with a corresponding inlet port 118. A second end of each channel is aligned with a corresponding outlet port 122. This creates a flow channel from an inlet port 118 to the corresponding outlet port 122 via the channel 114. The channels 114a-114c can also extend slightly beyond their respective inlet 118 and outlet ports 122 (not shown). The channels 114a-114c are sized to accept volumes, e.g., μL or mL, of the sample 120 containing cells to be adhered or captured in the respective regions 116 (See FIG. 2A). The channels 114a-114c may be further sized and shaped to affect adherence or capturing of the cells from the sample 120.

The base layer 130 provides structural support to the cell adherence region 116 and is formed of a sufficiently rigid, optically transparent, and gas impermeable material, such as poly(methyl methacrylate) (PMMA) or glass. The base layer 130 can have a suitable thickness, for example of about 0.1 mm to about 2 mm, or about 1.6 mm, determined by manufacturing and assembly restrictions.

The cover layer 150 contains the inlet ports 118 and outlet ports 122 used to feed the sample 120 in/out of the channel 114. The cover layer 150 thickness can be about 1 mm to about 10 mm, for example, about 3.6 mm, and is determined by the integration and assembly requirements. The inlet and outlet port 118, 122 diameters can be about 0.3 mm to about 3 mm, for example about 1 mm. The lower size limit is determined by the manufacturing restrictions. The upper size limit is determined by the desired flow conditions of sample 120 through the channel 114. In another example (not shown), a laser cutter can be used to cut a larger piece of PMMA into a desired size for the microfluidic device 10 and to cut holes for the inlet ports 118 and the outlet ports 122.

The intermediate layer 140 can be formed of a material that adheres to both the base layer 130 and the cover layer 150, such as a double sided adhesive (DSA) polyester layer. Each channel 114 can be formed, for example, by laser cutting polygons, such as rectangular sections, in the intermediate layer 140, which can itself be laser cut to the desired size, e.g., the size of the base layer 130. The height or depth of each channel 14 can be determined by the thickness of the intermediate layer 140, which is discussed in greater detail below.

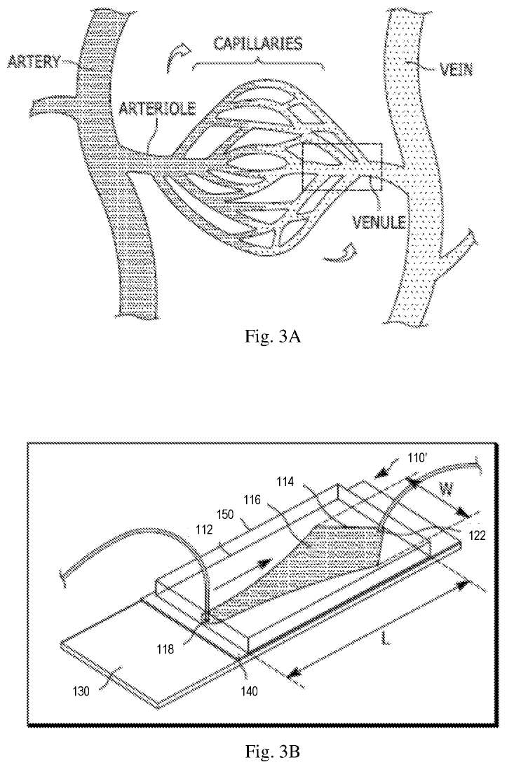

The intermediate layer 140 is adhered to the base layer 130 after each channel 114 is cut in the intermediate layer. The cover layer 150, which can have the same lateral dimensions as the base layer 30 and the intermediate layer 140, can be adhered onto the exposed side of the intermediate layer 140, thereby enclosing each channel 114. In the examples depicted in FIGS. 3A and 2B, the microfluidic device 110 is oriented such that the cover layer 150 is on top. Alternatively, the microfluidic device 110 can be oriented such that the cover layer 150 is on the bottom (not shown).

By of example, the microfluidic biochip shown in FIGS. 2A and 2B can constructed using PMMA cover layers, which were prepared by cutting an inlet and outlet port (0.61 mm in diameter and 26 mm apart) using a VersaLASER system (Universal Laser Systems Inc., Scottsdale, Ariz.). Double sided adhesive (DSA) film (iTapestore, Scotch Plains, N.J.) can be used as the intermediate layer and be cut to fit the size of the PMMA part. 28×4 mm microchannels 50 μm deep can be formed along the length of the DSA. DSA can then be attached to the PMMA cover layer to position the inlet and outlet ports between the DSA film outline. A Gold Seal glass slide can as the base layer and be assembled with the PMMA-DSA structure to form a biochip having a microfluidic channel.

FIG. 3B illustrates another example microfluidic biochip device 110′. The microfluidic device 110′ includes a housing 112 defining a single channel 114 having cell adhesion or adherence regions 116. The channel 114 is fluidly connected to an inlet port 118 at one end and an outlet port 122 at another end. Although FIG. 3B depicts only one channel 114, the microfluidic device 110′ can include multiple channels. The channel 114 receives a sample 20 from a patient. The channel 114 can have a length L of about 45-50 mm, a depth of about 50-57 μm (±1 μm), and a width W that varies along the length L from about 4 mm (at the inlet end) to about 16 mm (closer to the outlet end).

The geometry of the channel 114 in the microfluidic device 110′ is such that, when fluid is introduced into the channel 114, shear stress in the fluid flow along the longitudinal axis of the channel varies linearly along the channel length L. In one example, the shape of the channel 114 is such that the shear stress in the fluid flow along the axis of the chamber decreases linearly along the channel length L. To this end, the channel 114 can have a tapered, triangular, trapezoidal and/or diamond-shaped configuration. This allows for cell adhesion analysis over a range of shear stresses in a single experiment. Consequently, the configuration of the channel 114 allows for the study of the effect of flow conditions on the attachment of cells of interest, e.g., RBCs, to the surface of the channel defining the cell adhesion region 116.

In some examples, the microfluidic device 110, 110′ geometry and dimensions are determined to accommodate a uniform, laminar flow condition for the fluid sample 120, which determines capture efficiency and flow rate. In such examples, the channel 114 width W can vary from about 1 mm to about 15 mm. The minimum width W is determined by the diameters of the inlet and outlet port 118, 122. The upper limit width W is determined by the flow characteristics of fluid sample 120 in a confined channel 114. The channel 114 length L can be about 4 mm to about 100 mm. The lower channel 114 length L dimension is determined by the flow characteristics of the fluid sample 120 in a confined channel The upper limit length L is determined by cell capture efficiency. The channel 114 height/depth can be about 10 μm to about 500 μm, for example, about 50 μm, which is determined by fluid mechanics laws and constraints and flow characteristics of the fluid sample 120 in a confined channel. In any case, the channel(s) 114 in either device 110, 110′ can be a microchannel sized to receive and capable of testing a fluid sample 120 on the μL scale in volume.

In each microfluidic device 110, 110′ each cell adherence regions 116 can include a surface on which is provided a layer or coating of the at least one capturing agent. The at least one capturing agent can be, for example, a bioaffinity ligand. The same or different bioaffinity ligand 16 can be provided in each channel 114. The bioaffinity ligand can include, for example, at least one of E-Selectin, P-Selectin, intracellular adhesion molecule 1 (ICAM-1) and vascular cellular adhesion molecule 1 (VCAM-1) for potentially adhering cells, such as WBCs and/or RBCs, under physiological relevant shear stress and normoxia and hypoxia conditions and for detecting cell adhesion, such as WBC adhesion and/or RBC adhesion. The capturing agent can also include, for example, cells, such as endothelial cells, including human umbilical vein endothelial cells and human pulmonary microvessel endothelial cells for detecting cell adhesion, such as WBC adhesion and/or RBC adhesion, and cell deformability and morphology, under physiological relevant shear stress and normoxia and hypoxia conditions.

The capturing agent or bioaffinity ligand can be adhered to, functionalized or chemically functionalized to the cell adhesion region 116 of each channel 114. The bioaffinity ligands may be functionalized to the cell adhesion region 16 covalently or non-covalently. A linker can be used to provide covalent attachment of a bioaffinity ligand to the cell adhesion region 116. The linker can be a linker that can be used to link a variety of entities.

In some examples, the linker may be a homo-bifunctional linker or a hetero-bifunctional linker, depending upon the nature of the molecules to be conjugated. Homo-bifunctional linkers have two identical reactive groups. Hetero-bifunctional linkers have two different reactive groups. Various types of commercially available linkers are reactive with one or more of the following groups: primary amines, secondary amines, sulphydryls, carboxyls, carbonyls and carbohydrates. Examples of amine-specific linkers are bis(sulfosuccinimidyl) suberate, bis[2-(succinimidooxycarbonyloxy)ethyl]sulfone, disuccinimidyl suberate, disuccinimidyl tartarate, dimethyl adipimate 2HCl, dimethyl pimelimidate 2HCl, dimethyl suberimidate HCl, ethylene glycolbis-[succinimidyl-[succinate]], dithiolbis(succinimidyl propionate), and 3,3′-dithiobis(sulfosuccinimidylpropionate). Linkers reactive with sulfhydryl groups include bismaleimidohexane, 1,4-di-[3′-(2′-pyridyldithio)-propionamido)]butane, 1-[p-azidosalicylamido]-4-[iodoacetamido]butane, and N-[4-(p-azidosalicylamido)butyl]-3′-[2′-pyridyldithio]propionamide. Linkers preferentially reactive with carbohydrates include azidobenzoyl hydrazine. Linkers preferentially reactive with carboxyl groups include 4-[p-azidosalicylamido]butylamine.

Heterobifunctional linkers that react with amines and sulfhydryls include N-succinimidyl-3-[2-pyridyldithio]propionate, succinimidyl[4-iodoacetyl]aminobenzoate, succinimidyl 4-[N-maleimidomethyl]cyclohexane-1-carboxylate, m-maleimidobenzoyl-N-hydroxysuccinimide ester, sulfosuccinimidyl 6-[3-[2-pyridyldithio]propionamido]hexanoate, and sulfosuccinimidyl 4-[N-maleimidomethyl]cyclohexane-1-carboxylate. Heterobifunctional linkers that react with carboxyl and amine groups include 1-ethyl-3-[3-dimethylaminopropyl]-carbodiimide hydrochloride. Heterobifunctional linkers that react with carbohydrates and sulfhydryls include 4-[N-maleimidomethyl]-cyclohexane-1-carboxylhydrazide HCl, 4-(4-N-maleimidophenyl)-butyric acid hydrazide.2HCl, and 3-[2-pyridyldithio]propionyl hydrazide.

In some embodiments, a surface layer of 3-aminopropyl triethoxy silane (ATES) and/or (3-mercaptopropyl)trimethoxysilane (MTPMS) can be initially applied to surfaces of the microchannel followed by incubation with N-γ-maleimidobutyryl-oxysuccinimide ester (GMBS) to functionalize the bioaffinity ligand or capturing agent to the surfaces.

By way of example, a GMBS working solution can prepared by dissolving GMBS in DMSO and diluting with ethanol. A bioaffinity ligand described herein, such as E-Selectin, P-Selectin, ICAM-1, or VCAM-1 can be diluted with PBS to create a bioaffinity ligand working solution. The GMBS working solution can injected into the microchannels twice and incubated at room temperature. Following GMBS incubation, the microchannels can be washed. Next, the bioaffinity ligand working solution can injected into the microchannels and incubated at room temperature. The surface can then passivated by injecting a BSA solution incubated overnight at 4° C., thereby forming a bioaffinity ligand functionalized glass surface. The microchannels can be optionally rinsed with PBS before processing samples.

Alternatively, the bioaffinity ligands may be non-covalently coated onto the cell adhesion region 16. Non-covalent deposition of the bioaffinity ligand to the cell adhesion region 16 may involve the use of a polymer matrix. The polymer may be naturally occurring or non-naturally occurring and may be of any type including but not limited to nucleic acid, e.g., DNA, RNA, PNA, LNA, and the like or mimics, derivatives or combinations thereof, amino acid, e.g., peptides, proteins (native or denatured), and the like or mimics, derivatives or combinations thereof, lipids, polysaccharides, and functionalized block copolymers. The bioaffinity ligand may be adsorbed onto and/or entrapped within the polymer matrix. Alternatively, the bioaffinity ligand may be covalently conjugated or crosslinked to the polymer, e.g., it may be “grafted” onto a functionalized polymer.

An example of a suitable peptide polymer is poly-lysine, e.g., poly-L-lysine. Examples of other polymers include block copolymers that comprise polyethylene glycol (PEG), polyamides, polycarbonates, polyalkylenes, polyalkylene glycols, polyalkylene oxides, polyalkylene terepthalates, polyvinyl alcohols, polyvinyl ethers, polyvinyl esters, polyvinyl halides, polyvinylpyrrolidone, polyglycolides, polysiloxanes, polyurethanes, alkyl cellulose, hydroxyalkyl celluloses, cellulose ethers, cellulose esters, nitrocelluloses, polymers of acrylic and methacrylic esters, methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), polyvinyl acetate, polyvinyl chloride, polystyrene, polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate), poly(lactide-glycolide), copolyoxalates, polycaprolactones, polyesteramides, polyorthoesters, polyhydroxybutyric acid, polyanhydrides, poly(styrene-b-isobutylene-b-styrene) (SIBS) block copolymer, ethylene vinyl acetate, poly(meth)acrylic acid, polymers of lactic acid and glycolic acid, polyanhydrides, poly(ortho)esters, polyurethanes, poly(butic acid), poly(valeric acid), and poly(lactide-cocaprolactone), and natural polymers such as alginate and other polysaccharides including dextran and cellulose, collagen, albumin and other hydrophilic proteins, zein and other prolamines and hydrophobic proteins, copolymers and mixtures thereof, and chemical derivatives thereof including substitutions and/or additions of chemical groups, for example, alkyl, alkylene, hydroxylations, oxidations, and other modifications routinely made by those skilled in the art.

In some embodiments, where the capturing agent is a cell, at least one surface of the microchannel can be coated with a bioadhesive molecule, e.g., protein or glycoprotein, to which the cells attaches. For example, assembled microchannels can be coated GMBS and thereafter loaded with Fibronectin to adhere the fibronectin to the microchannel surface. Human endothelial cells can then be seeded on the microchannel surface at, for example, a density of about 4×10⁶ cells/mL to allow cell attachment and spreading. The seed cells can be culture under static conditions or under flow. To accomplish cell culture under flow, the microchannels can be coupled to a pump a reservoir that contains culture medium which is circulated through the microchannel at controlled environment, e.g., 37° C. and CO₂ concentration of 5% unless the culture solution is supplemented with HEPES buffer solution, in which case CO₂ is not be needed.

In some examples, each channel 114 can include multiple, separate cell adhesion regions 116 functionalized with at least one bioaffinity ligand. At least two or at least three of the channels 114 can include different bioaffinity ligands. In other examples, the plurality of channels 114 can include the same bioaffinity ligands.

In still other examples, at least one channel 114 can include at least two different bioaffinity ligands functionalized on the cell adhesion region 116. The different bioaffinity ligands can be located at different positions within the cell adhesion region 116 of each channel 14. For example, at least one of E-Selectin, P-Selectin, ICAM-1, VCAM-1, or other bioaffinity ligands, such as fibronectin, laminin, and thrombospondin, can be localized at different positions along the length L of the at least one channel 114.

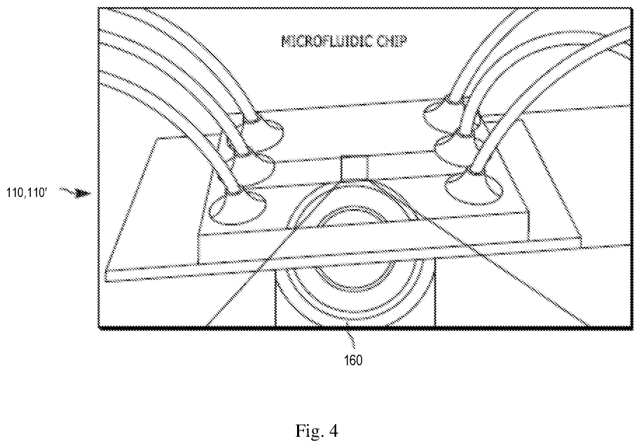

In some examples, a fluid sample 120, which includes at least one blood cell from a subject is introduced into each channel 114. The capturing agent or bioaffinity ligand can bind cells of interest in the fluid sample to a surface or wall(s) of the microchannel along the cell adhesion region 116. The quantity of blood cells bound to the microchannel walls by the capturing agent can be imaged using an imaging system 160 (see FIG. 4). The imaging system 160 can determine, for example, the aspect ratio (AR) of the blood cells as well as quantify membrane, cellular and adhesive properties of the blood cells to monitor disease severity, upcoming pain crisis, treatment response, and treatment effectiveness in a clinically meaningful way.

In some examples, the imaging system 160 can be a lens-based imaging system or a lensless/mobile imaging system. In some embodiments, the lensless imaging system 160 can be a CCD sensor and a light emitting diode. By way of example, as shown in FIG. 4, a fluorescent microscopy camera (EXi Blue EXI-BLU-R-F-M-14-C) and an Olympus IX83 inverted, fluorescent motorized microscope with Olympus Cell Sense live-cell imaging and analysis software can be used to obtain real-time microscopic images. Olympus (20x/0.45 ph2 and 40x/0.75 ph3) long working distance objective lenses can be utilized for phase contrast imaging of cells adhered in the microchannels. During real-time microscope imaging and high resolution video recording at 7 fps rate, controlled fluid flow with stepwise increments can be applied until cell detachment from the microchannel surface is observed. Videos were converted to single frame images for further processing and analysis. The cell dimensions can then analyzed by using Adobe Photoshop software (San Jose, Calif.).

In some examples, a mobile imaging and quantification algorithm can be integrated into or with the microfluidic device 110, 110′. The algorithm can achieve reliable and repeatable test results for data collected in all resource settings of the microfluidic device 110, 110′.

In other examples, the microfluidic device 110, 110′ can be configured to cooperate with a cellular phone having imaging capabilities. In such a case, the cellular phone can be provided with or capable of obtaining image analysis algorithms/software, e.g., via an online application. Images can be recreated by the cellular phone camera software and loaded into a custom phone application that identifies adhered RBCs, quantifies the number of adhered RBCs in the image, and displays the results.

The cells of interest can be blood cells obtained from the subject and the imaging system 160 can quantify the adhered cells in each respective channel 114 to monitor the health of a subject from which the cells are obtained. In other examples, the imaging system 160 can quantify the adhered cells in each respective channel 114 to monitor the progression of a disease, such as SCD, of a subject from which the cells are obtained. In still other examples, the imaging system 160 can quantify the adhered cells in each channel 114 to measure the efficacy of a therapeutic treatment administered to a subject from which the cells are obtained.

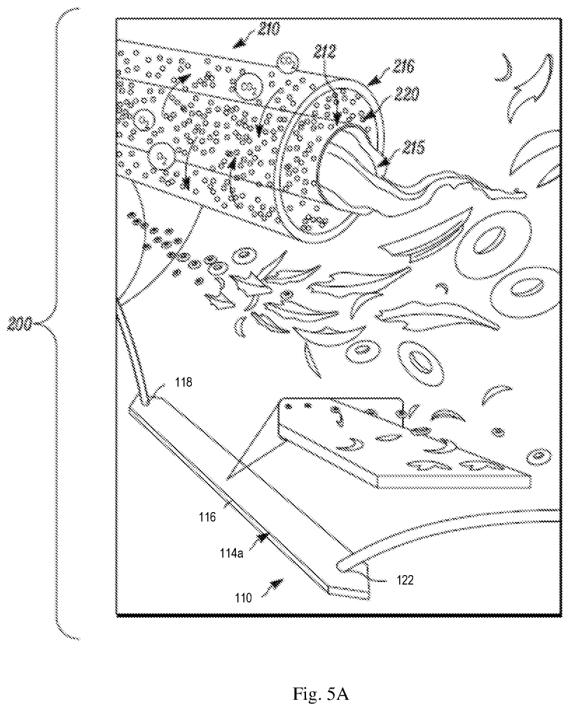

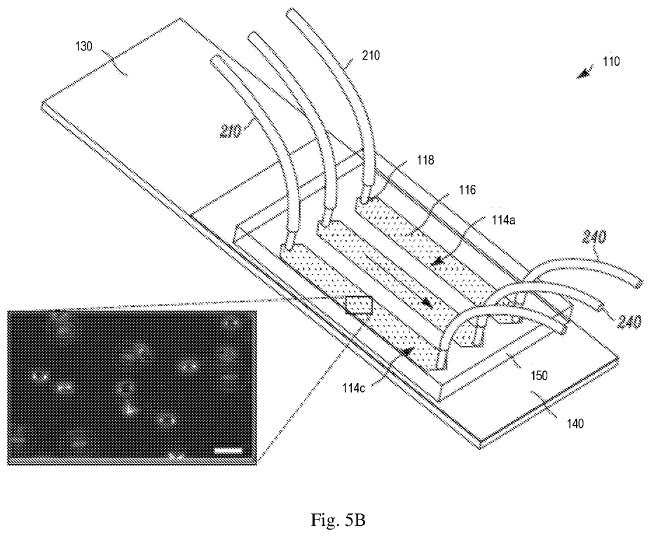

FIGS. 5A-5C illustrate a microfluidic system 200 in accordance with another embodiment described herein that includes a microfluidic device 110 as previously described and at least one micro-gas exchanger 210. The microfluidic system 200 can be used to adjust the oxygen tension in the blood sample prior to introduction thereof into the microchannels 114a-114c. Blood is deoxygenated at the micro-gas exchanger during flow and reached the microchannels 114a-114c with RBCs adhering to the functionalized cell adhesion surfaces. The micro-gas exchanger allows for easy adaptation to portable point of care (POC) microfluidic systems.

The microfluidic device 200 integrated with a micro-gas exchanger 210 described herein allows interrogation and manipulation of biological fluid at a single-cell level while being clinically feasible, cost and labor efficient, and easily implementable. The microfluidic system eliminates the intricate microchannel design and configuration required in PDMS based systems by controlling the oxygen tension of the biological fluid before it reaches the microchannel The microfluidic system can be used to analyze the adhesion of WBCs and/or RBCs in blood samples of patients with hematological disorders, such as SCD, where oxygen tension control is desirable.

The micro-gas exchanger 210 includes concentric inner and outer tubes 212, 216. The inner tube 212 has a gas-permeable wall 214 defining a central passage 215 extending the entire length of the inner tube. The outer tube 216 has a gas impermeable wall 218 defining a central passage extending the entire length of the outer tube. An annular space 220 is formed between the tubes 212, 216. The central passage 215 receives the blood sample 20 and is in fluid communication with one or more inlet ports 18 of the microfluidic device 110. As shown, each inlet port 118 is fluidly connected to a different micro-gas exchanger 210 to specifically tailor the blood delivered to each microchannel 114a-114c. An outlet tube 240 is connected to each outlet port 122. A controlled gas flow takes place in the annular space 220 and blood flows inside the inner tube 212. The deoxygenation of blood occurs due to gas diffusion (5% CO₂ and 95% N₂) through the inner gas-permeable wall 214.

To setup the micro-gas exchanger 210, medical grade gas-permeable silicone tubing 212 (300 μm inner diameter (ID)×640 μm outer diameter (OD), Silastic Silicone Laboratory Tubing, Dow Corning) can be placed inside the impermeable tubing 216 (1600 μm ID×3200 μm OD, FEP tubing, Cole-Parmer). The gas permeability of the outer FEP tubing 216 (0.59 Barrer for CO₂; 1.4 Barrer for O₂) can be less than 0.2% of the inner silicone tubing 212 (2000 Barrer for CO₂; 800 Barrer for O₂) for both CO₂ and O₂. Due to this construction, the blood sample can exchange gases through the permeable inner tubing wall 214 with 5% CO₂ and 95% N₂ controlled gas inside the impermeable tubing annular space 220 by diffusion.

By way of example, in operation, the blood sample can be injected with the syringe pump (NE300, New Era Pump Systems) into the system 200 at 18.5 μL/min to fill the tubing passage 215 and the microchannels 114a-114c, and at 1.85 μL/min to impose about 1 dyn/cm² of shear stress while flowing in the functionalized microchannels. After flowing 15 μL of blood through the microchannels 114a-114c, flow cytometry staining buffer (FCSB, 1X) can be inserted to wash the microchannels at 10 μL/min, which imposed 1 dyn/cm² of shear stress, making the adhered RBCs visible through the inverted microscope (Olympus IX83) and microscopy camera (EXi Blue EXI-BLU-R-F-M-14-C) for high-resolution images. At least 180 μL of FCSB can be used to wash the microchannels 114a-114c and the images can be taken during buffer flow. For the entire procedure, the medium flowing in each microchannel 114a-114c can be deoxygenated by a micro-gas exchanger 210 to achieve physiological flow and hypoxic conditions.

It can be expected that a microfluidic device 110, 110′ platform disclosed herein is applicable to the study of single cell heterogeneity of adherent cells within subjects in larger clinically diverse populations and may provide important insights into complex disease phenotypes other than SCD. For example, abnormal WBC and/or RBC adhesion to microvascular surfaces has previously been implicated in other multi-system diseases, such as β-thalassemia, diabetes mellitus, hereditary spherocytosis, polycythemia vera, and malaria.

By way of example, referring back to FIG. 3A, sickled RBC adherence to blood vessel walls has been shown to take place in post-capillary venules. To this end, this application contemplates a microfluidic SCD biochip including at least one microchannel having a width W of approximately 60 μm, a depth of about 50 μm, and fluid flow velocities within a range of approximately 1-13 mm/sec—similar to that reported for post-capillary venules.

In one example, the microfluidic biochip 110, 110′ can be used in an SCD testing method utilizing pathophysiologic correlates, including but not limited to, analyses of adhesion and membrane properties in HbSS and HbSC, at baseline and during vaso-occlusive crises, with treatment, and in the presence of end-organ damage. The SCD testing method described herein can be completed in less than ten minutes. In some examples, the SCD testing method provides a highly specific analyses of CMA properties in RBCs, WBCs, circulating hematopoietic precursor cells and circulating endothelial cells. In one example, the SCD testing method is performed using a portable, high efficiency, microfluidic biochip and a miniscule blood sample (<15 μL). The SCD testing method can provide a sophisticated and clinically relevant strategy with which patient blood samples may be serially examined for cellular/membrane/adhesive properties during SCD disease progression.

In some examples, the microfluidic device 110, 110′ can accurately quantitate cellular interactions and membrane properties using a single drop of blood. The biochip and method may validate insights about mechanisms of disease in SCD and may reveal correlations between disease heterogeneity and acute and/or chronic SCD complications.

The microfluidic device 110, 110′ can also evaluate membrane and cellular abnormalities by interrogating a number of recognized abnormalities in a range of clinical phenotypes. To date, these phenotypes are discussed in various correlative SCD studies ranging between clinical reports, testing results, interventions, and/or chart reviews.

The examples described herein have advantages because existing conventional methods cannot assess longitudinal and large-scale SCD clinical correlations with cellular, membrane, and adhesive properties. To this end, this application contemplates a method for using an SCD biochip for examining cellular properties and interactions. These cellular properties and interactions include, but are not limited to, RBC cellular and adhesive properties, WBC cellular and adhesive properties, circulating endothelial characteristics and hematopoietic precursor cell characteristics. For example, a microfluidic biochip can include a plurality of microchannels that are functionalized with lignin, selectins, avidin and/or biotinylated antibodies to BCAM/LU, CD11b, CD34, and/or CD146. A method is contemplated for correlating SCD biochip function in heterogeneous SCD populations, including but not limited to, HbSS and HbSC over a range of ages, and in those with acute and chronic complications and compared with normal HbAA controls.

A simple test for adhesion would allow exploration of its role in chronic complications in SCD, in addition to during crisis. Selectins may be tested using microfluidic biochips as an adhesive surface, in place of cultured endothelial cells. Endothelial selectins are believed to mediate leukocyte adhesion and rolling on the endothelial surface. For example, in experimental models of SCD, P-selectin is widely expressed on vascular endothelium and endothelial E-selectin is important for vascular occlusion. This application contemplates a microfluidic SCD biochip including at least one microchannel provided with at least one immobilized P-selectin, E-selectin, ICAM-1, and/or VCAM-1 adhesion.

### EXAMPLES

### Example 1

Sickle cell disease (SCD) is characterized by frequent and unpredictable vaso-occlusive crises (VOC), which can lead to acute pain, chronic organ damage, and can contribute to early mortality. Previous studies have revealed a multistep and multicellular paradigm in VOC, which involves activated leukocytes adhering to activated endothelium and circulating sickle erythrocytes, thus hindering blood flow and contributing to VOC propagation. Multiple adhesion receptor/ligand pairs have been identified in these multicellular interactions. P-selectin is known to mediate platelet activation, coagulation, and inflammation, and has been found, of all selectins, to be the most essential for initiation and maintenance of the cascade of events triggered by leukocyte adhesion to vascular endothelium during VOC. In vitro and in vivo models showed that inhibition of P-selectin-mediated adhesion pathways significantly reduced leukocyte adhesion to activated endothelium and ameliorated impaired blood flow in SCD. Crizanlizumab is a humanized monoclonal antibody against P-selectin, which, when infused monthly, reduced the annual rate of VOC in patients with SCD in randomized placebo-controlled clinical trials. However, currently, there is no in vitro model for this effect, largely due to the lack of a universally accepted, standardized physio-logic flow-based adhesion assay with which to measure leukocyte adhesion to P-selectin.

In this example, we describe a standardized microfluidic whole blood adhesion assay using the P-selectin SCD Biochip platform and clinically available whole blood samples to measure leukocyte adhesion to P-selectin under physiologic flow conditions. Such an assay could help visualize cellular adhesion characteristics before and after therapeutic interventions, and may reveal the association between patient-specific adhesion profiles and clinical phenotypes. Indeed, data reported in this study support the inhibitory effect of pre-emptive Crizanlizumab on P-selectin mediated leukocyte adhesion, and of post-treatment Crizanlizumab on leukocyte detachment.

### Materials and Methods

All anonymized blood samples were collected in ethylenediaminete-traacetic acid (EDTA) from individuals with homozygous SCD (HbSS) seen at University Hospital's Hematology and Oncology Division. Consent was acquired from all individuals on an IRB-approved protocol. Clinical information, including white blood cell count (WBC, 109/L), platelet count (109/L), absolute neutrophil count (ANC, 106/L), reticulocyte count (109/L), lactate dehydrogenase levels (LDH, U/L), ferritin levels (μg/L), hemoglobin A (HbA) %, hemoglobin S (HbS) %, hemoglobin F (HbF) %, and total hemoglobin (g/dL), was obtained from the electronic medical record.

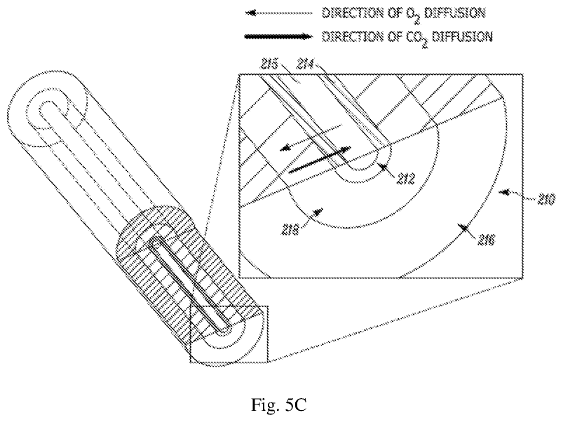

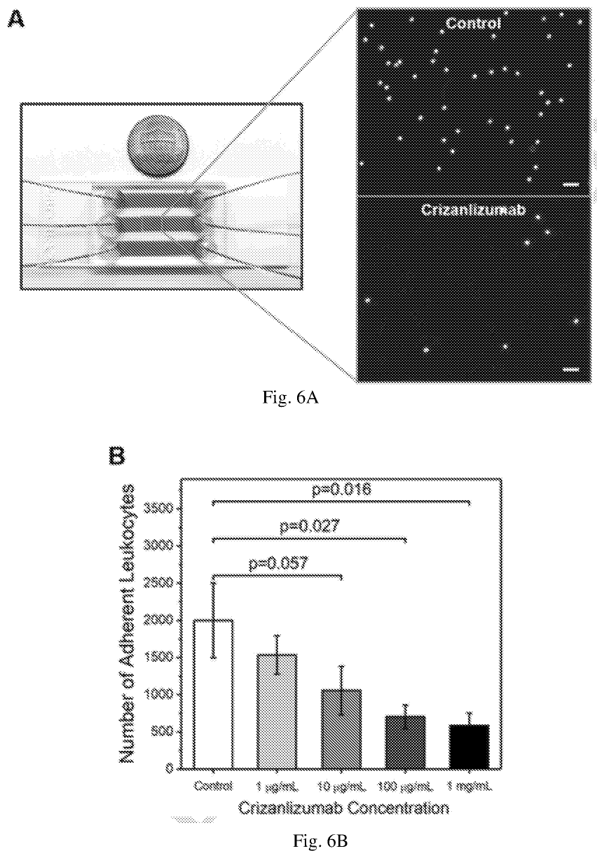

Microfluidic devices were fabricated using a lamination technique. Each microfluidic device contained 3 identical microchannels with a height of 50 μm (FIG. 6A), which were designed to recapitulate volume and flow of post-capillary venules. The microchannels were functionalized with recombinant human P-selectin (25 μg/mL) and blocked with 2% bovine serum albumin (BSA) to pre-vent non-specific adhesion. After tubing assembly, whole blood samples were mixed with Hanks' buffer modified with calcium and magnesium (1:1 v/v), and 15 μL of the diluted whole blood was injected into the microchannels using New Era NE-300 syringe pump system (Farming-dale, N.Y.) at an approximate shear rate of 1 dyne/cm². Non-adherent cells were rinsed out by flowing Hanks' buffer. Phase-contrast images of the microchannels with adherent leukocytes were recorded at 10× with an inverted microscope (Olympus IX83) and a camera (EXi Blue EXI-BLU-RF-M-14-C), and adherent leukocytes were manually quantified with Adobe Photoshop software (San Jose, Calif.) in a 32 mm² area.

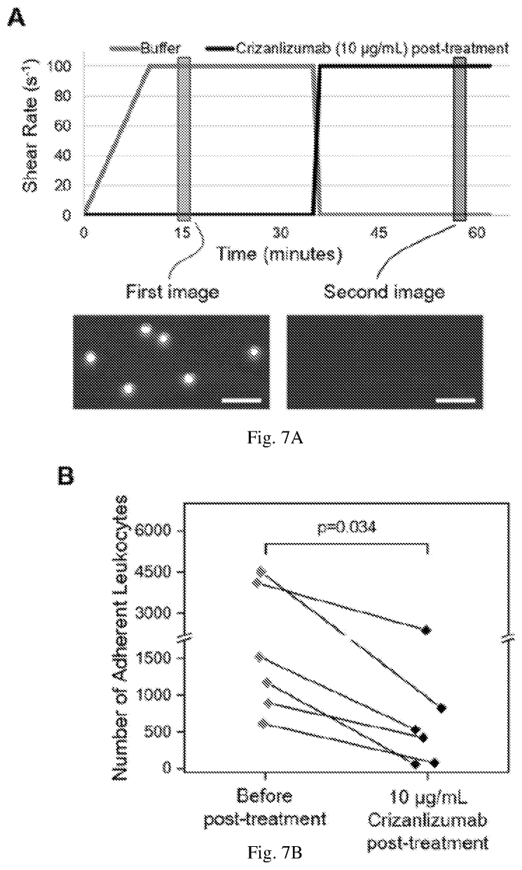

Crizanlizumab (ADAKVEO®, SEG101) stock solution (10 mg/mL) was donated by Novartis (Basel, Switzerland). For the inhibition experiments, P-selectin immobilized-microchannels were pre-treated with Hanks buffer as vehicle control, or Crizanlizumab with graded concentrations of 1, 10, 100 μg/mL, or 1 mg/mL at 37° C. for 30 min. There-after, the adhesion assay was performed. Human IgG2 (Sigma Aldrich, St. Louis, Mo.), at 100 μg/mL, was included as the isotype control for Crizanlizumab. For the detachment experiments, modified microchannels with individual inlets for separately injecting blood and drug-containing buffer were designed to allow transition from washing buffer to Crizanlizumab during rinsing. Two synchronous pumps connected to these inlets were programmed such that Crizanlizumab at 10 μg/mL was injected into microchannels after Hanks buffer without a disruption in shear rate. The programmed flow rates of the two synchronous pumps over the course of the experiment are shown in FIG. 7A, which insured a constant shear rate in the microchannels. Cell behaviors were also continuously recorded. Phase contrast images of the adherent leukocytes were collected during Hanks's buffer rinse and Crizanlizumab flow for comparison.

Leukocyte adhesion data were reported as mean±standard error of the mean (SEM). Clinical variables of the study population were re-ported as mean±standard deviation (SD). Statistical analyses were per-formed with Minitab (Minitab Inc., State College, Pa.). A test for normality was initially performed on relevant variables. A paired t-test was per-formed to compare paired groups before and after Crizanlizumab treatment. Statistical significance was determined based on a p-value<0.05 (p<0.05).

### Results

In this example, we examined the effect of Crizanlizumab on leukocyte adhesion to P-selectin in two distinct ways. First, the P-selectin immobilized microchannels were pre-treated with Crizanlizumab at varying concentrations prior to the adhesion experiments to analyze the utility of the drug in preventing leukocyte adhesion. In the second approach, the adhesion assay was performed first, followed by treatment with Crizanlizumab at a fixed concentration to assess whether it would reverse already-established adhesive interactions between leukocytes and P-selectin.

These assays revealed that leukocytes from individuals with HbSS SCD displayed significant and heterogeneous adhesion to immobilized P-selectin under physiologic flow conditions in the absence of Crizanlizumab treatment (N=8). Crizanlizumab pre-treatment of P-selectin functionalized microchannels led to a dose-dependent reduction in leukocyte adhesion. Leukocyte adhesion was significantly lower in the microchannels pre-treated with Crizanlizumab at concentrations of 100 μg/mL or 1 mg/mL, compared to non-Crizanlizumab controls (FIG. 6B, 100 μg/mL: 703±161, range: 131-1474, p=0.027; 1 mg/mL:587±168, range: 81-1403, p=0.016; control: 1998±503, range: 4256-526, paired t-test) while the significance was borderline for the concentration of 10 μg/mL (FIG. 6B, 1057±325, range: 195-2948, p=0.057, paired t-test). Leukocyte adhesion in the 1 μg/mL Crizanlizumab treated microchannels was not significantly different compared to the non-treated microchannels (FIG. 6B, 1535±258, range: 257-2732, p>0.05, paired t-test). Furthermore, the isotype control did not show an inhibitory effect comparable with Crizanlizumab (2290±849 vs 596±221, N=3) at 100 μg/mL. Of note, these assays were performed using blood samples from on-transfusion individuals.

We also assessed blood samples from non-transfusion individuals (on-hydroxyurea or supportive care) using the P-selectin microchannels with 1 mg/mL Crizanlizumab pre-treatment, similar inhibitory effect was observed (1 mg/mL Crizanlizumab vs control: 179±79 vs 327±101, range: 39-691 vs 74-742, N=8, p=0.063, paired t-test).

Our results also demonstrated that post-treatment of leukocytes with Crizanlizumab, after the leukocytes adhered to the immobilized P-selectin, resulted in detachment of rolling leukocytes whereas firmly attached cells remained adherent. As shown in FIG. 7B, there was a significant decrease in leukocyte adhesion before and after introducing Crizanlizumab into the microchannels (719±354 vs 2138±700, range: 613-4510 vs 58-2388, N=6 (4 out of which were on-transfusion), p=0.034, paired t-test). These findings support the role of Crizanlizumab in preventing VOC, by blocking leukocyte adhesions; however, Crizanlizumab may also mitigate a VOC due to its ability to reverse some, but not all, established aberrant adhesive interactions.

Previous studies have shown that Crizanlizumab significantly re-duces the frequency of pain crises in SCD and decreases the annual rate of hospitalized days. In this example, using the P-selectin SCD Biochip, we showed that under physiologic flow conditions, Crizanlizumab significantly ameliorates leukocyte adhesion to immobilized P-selectin. Importantly, Crizanlizumab applied after adhesion, impacted rolling leukocytes, although firmly-adherent leukocytes were unresponsive. This may, at least in part, explain the in vivo heterogeneity of Crizanlizumab reported previously.

### Example 2

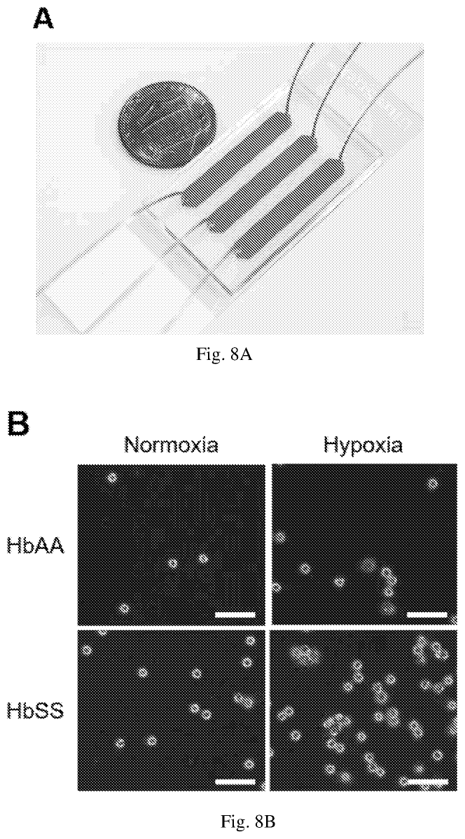

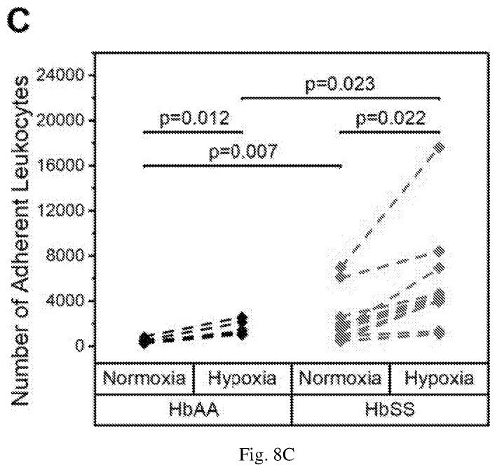

This example describes a microfluidic device that includes an E-selectin-functionalized microchannel coupled with a displacement pump that provides a physiologically relevant shear stress value of 1 dyne/cm² to flow the blood sample contained in a syringe (FIG. 8A). The microchannels are connected to the syringe by the inlet silicon tubing and mounted on the stage of an inverted microscope. Normoxia is controlled by flowing ambient-exposed blood samples. Hypoxia is created using a micro-gas exchanger, which facilitates oxygen exchange and results in a SpO₂ of 83% in the blood flow. A phase-contrast image is captured under 10× magnification in the middle of the microchannel via a charge-coupled device (CCD) camera, and adherent leukocytes are quantified in a window of 32 mm².

### Test Method

### Standardized Microfluidic Adhesion Assay

The assembled microfluidic channels are rinsed with absolute ethanol and PBS (1×), which are then functionalized with human recombinant E-selectin, and passivated with 2% bovine serum albumin (BSA). Prior to injection, ethylenediaminetetraacetic acid (EDTA)-anticoagulated whole blood samples are re-calcified with Hank's buffer (1:1 v/v). A 15 μl of mixed blood sample is flowed across the microchannel at a constant flow rate and non-adherent cells are washed off by Hank's buffer. The adherent leukocytes are manually quantified.

### Assessment of Leukocyte Adhesion to E-Selectin Under Normoxia or Hypoxia

The normoxic condition is controlled by flowing ambient-exposed blood samples. To create a physiologic hypoxic condition in the blood flow, a micro-gas exchanger is fabricated and employed, which consists of a medical grade gas-permeable silicone tubing placed inside an impermeable tubing, allowing oxygen exchange between the blood flow and the 5% CO₂ and 95% N₂-controlled gas through the permeable tubing wall inside the impermeable tubing. The oxygen exchange results in a SpO₂ of 83% in the blood flow in the microchannels.

To assess leukocyte adhesion to E-selectin under normoxia or hypoxia, 5 samples from 5 healthy donors (HbAA) and 11 samples from 11 subjects with homozygous SCD (HbSS) were tested using E-selectin functionalized microchannels. The results indicate that under both normoxia and hypoxia, HbSS subjects had significantly more leukocyte adhesion to E-selectin compared to HbAA subjects, and for both HbAA and HbSS subjects, hypoxia significantly enhanced leukocyte adhesion to E-selectin compared to normoxia (FIGS. 8B&C).

### Example 3

In this Example, we examined the adhesion of sickle RBCs from SCD subjects to immobilized ICAM-1 in microphysiological flow conditions utilizing an in vitro microfluidic adhesion assay. We demonstrated that ICAM-1 supports the adhesion of sickle RBCs, in a subject-specific manner; adhesion of RBCs from homozygous subjects (HbSS) strongly correlated with high-grade hemolysis and a history of intracardiac or intrapulmonary right-to-left shunts. In particular, RBCs from subjects with HbSS and evidence for hemolysis and inflammation (elevated lactate dehydrogenase [LDH] levels, absolute reticulocyte counts [ARCs], and WBC count) have an increased propensity for adhesion. Notably, sickle RBC adhesion to ICAM-1 was mediated via fibrinogen, which forms intercellular bridges. Lastly, we observed that a fraction of adherent RBCs exhibited rolling motion at high shear rates and did not disassociate from the immobilized protein, suggesting a novel mechanism by which RBCs may contribute to the initiation of VOC events in SCD.

### Methods

### Flow Adhesion Experiments

ICAM-1 functionalized microchannels of the microfluidic device were connected to blood-containing syringes through 40-cm inlet tubing. A total of 15 mL of whole blood sample was perfused into the microchannels at an approximate shear stress of 1 dyne/cm², corresponding to the typical shear stress observed in postcapillary venules, via a constant displacement syringe pump (New Era NE-300; New Era Pump Systems, Farmingdale, N.Y.). To remove the nonadherent cells, another syringe filled with wash buffer (1% bovine serum albumin and 0.09% sodium azide in 13 phosphate buffered saline [PBS]) was connected to the microchannels, and the buffer solution was injected at 10 mL/min, corresponding to a wall shear stress of 1 dyne/cm², until all nonadherent cells had been cleared. Phase-contrast images of the microchannel surface were obtained using an inverted microscope at 203. Quantification of ICAM-1-adherent RBCs was performed manually using Adobe Photo-shop CS5 (Adobe, San Jose, Calif.) within a rectangular field of view (fov) with a surface area of 32 mm².

### Inhibition Experiments

For the inhibition studies, whole HbSS blood samples were mixed with a monoclonal mouse antibody against human α₄β₁ or LFA-1 at a final antibody concentration of 50 mg/mL and incubated at 37° C. for 1 hour prior to the adhesion experiments. As a control, samples were treated with the same concentration of isotype-control mouse immunoglobulin G1 for the same duration. Anti-human LFA-1 antibody and the isotype-control mouse immunoglobulin G were from BioLegend (San Diego, Calif.). Anti-human α₄β₁ antibody was purchased from Abcam (Cambridge, Mass.). To block the β2-associated adhesion pathways, a recombinant human β2 protein solution (Novus Biologicals, Centennial, Colo.) was perfused into ICAM-1-functionalized microchannels and incubated for 1 hour at 37° C. Before starting the adhesion experiments, the microchannels were rinsed 3 times with PBS. Similarly, to test the inhibitory effect of fibrinogen (Enzo Life Sciences, Farmingdale, N.Y.) or low molecular weight heparin (LMWH; AMSBIO, Cambridge, Mass.), ICAM-1-functionalized microchannels were injected with fibrinogen (range, 1-20 mg/mL) or LMWH (range, 0.1-5 mg/mL). After an hour at 37° C., the microchannels were rinsed with PBS 3 times, and adhesion was assessed as above.

### Statistical Methods

Minitab 19 (Minitab, State College, Pa.) was used to analyze the results. A normality test was performed to determine whether the variables were normally distributed. Because of the largely non-normally distributed variables, Mann-Whitney U tests were used to compare 2 groups, and Kruskal-Wallis tests with Dunn's correction were used to compare multiple groups, unless stated otherwise. K-means clustering analysis was performed to identify distinct subject subpopulations based on hemolysis biomarkers. Through-out this article, the error bars represent±standard error of the mean (SEM). P<0.05 was considered statistically significant.

### Results

Abnormal Sickle RBC Adhesion to ICAM-1 is Heterogeneous in a Clinically Diverse Population with SCD

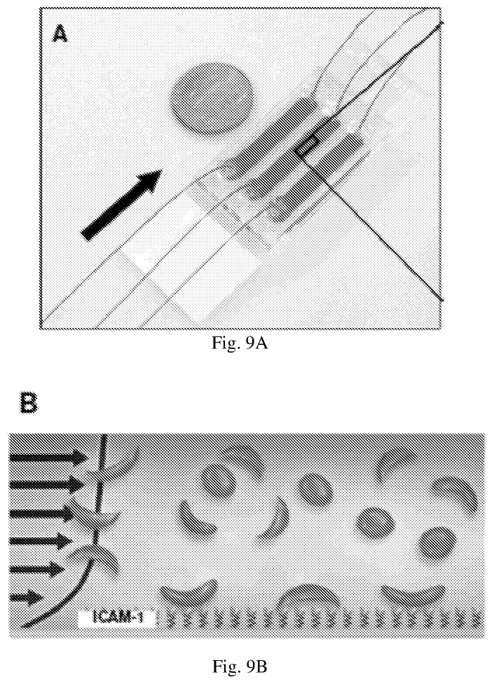

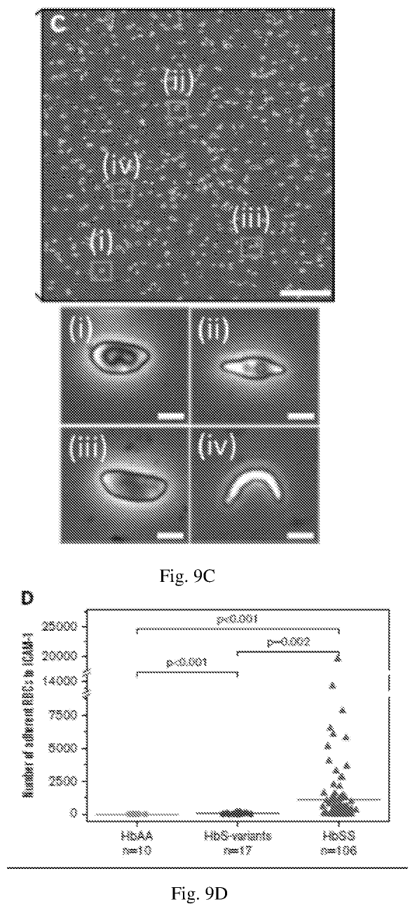

We analyzed the ability of RBCs to adhere to ICAM-1 using whole blood samples from individuals with (HbSS, HbS variant) or without (HbAA) SCD in a physiologically relevant in vitro microfluidic adhesion platform. Our results showed that HbSS RBCs had significantly greater adhesion to immobilized ICAM-1 than did HbS variant RBCs or HbAA RBCs (FIG. 9D; mean adhesion 6 standard deviation [SD] 5 1486±3312 per fov for HbSS, 54±59 per fov for HbS variant, and 3.5±1.4 per fov for HbAA, P<0.005). We also observed subject-specific and heterogeneous adhesion profiles for HbSS RBCs, ranging between 6 and 19 495 for the entire study population with homozygous SCD (n=55; mean adhesion 1486±3312 per fov).

Sickle RBC Adhesion to ICAM-1 is Associated with Clinical Biomarkers of Hemolysis

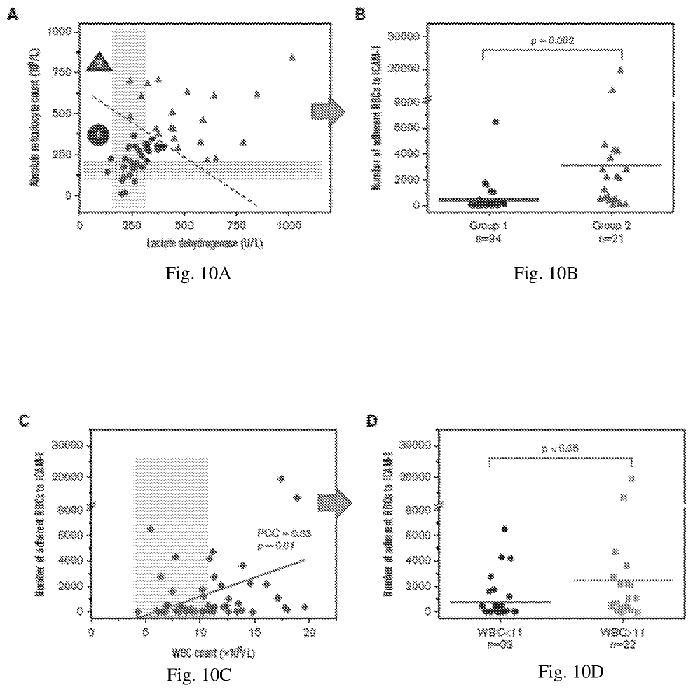

We found an association between HbSS RBC adhesion to ICAM-1 and clinical variables, in samples obtained at the clinical steady-state. We performed a k-means clustering analysis based on subject LDH levels and ARCs, which resulted in 2 distinct HbSS subgroups (FIG. 10A). We found that RBCs from samples in group 1 (n 5 34; lower LDH and lower ARC laboratory profiles) had significantly less adhesion to ICAM-1 compared with RBCs from samples in group 2 (n=21; higher LDH levels and higher ARC laboratory profiles) consistent with hemolysis (FIG. 10B; 453±199 per fov vs 3159±1038 per fov; mean adhesion 6 SEM, respectively; P=0.002). These findings suggest an association between RBC adhesion to the vascular bed, hemolysis, and shortened RBC half-life. In addition, we found an association between HbSS RBC adhesion to ICAM-1 and WBC count (FIG. 10C-D). Subjects with an elevated WBC count (>11×10⁹ per liter) exhibited significantly higher RBC adhesion compared with those with a normal WBC count (FIG. 10D; 2532±1017 per fov vs 789±265 per fov; mean adhesion 6 SEM, respectively; P=0.02). The clinical variables of subjects in group 1 and group 2 are summarized in Table 1.

TABLE 1
Clinical variables of the tested subjects with HbSS SCD

| | Group 1 (n 5 34) | Group 2 (n 5 21) | P |
|---|---|---|---|
| Age, y | 37.8 ± 13.6 | 35.9 ± 13.0 | .628** |
| Hemoglobin, g/dL | 8.6 ± 1.4 | 8.6 ± 1.3 | .965** |
| Mean corpuscular | 96.7 ± 14.2 | 92.6 ± 5.4 | .222* |
| volume, fL | | | |
| Platelets, 310⁹/L | 313.7 ± 140.9 | 364.3 ± 89.4 | .147* |
| WBC count, 310⁹/L | 9.6 ± 3.2 | 12.7 ± 3.8 | .002* |
| Absolute neutrophil | 5514 ± 2303 | 7359 ± 2841 | .014* |
| count, 310⁶/L | | | |
| ARC, 310⁹/L | 214.7 ± 89.1 | 482.2 ± 174.9 | <.001* |
| LDH, U/L | 271.9 ± 64.0 | 510.0 ± 200.2 | <.001* |
| Ferritin, mg/L | 2680.2 ± 2626.3 | 1920.4 ± 2179.3 | .472* |
| Hemoglobin S, % | 58.5 ± 23.7 | 56.5 ± 20.2 | .523* |
| Hemoglobin A, % | 25.8 ± 24.6 | 30.0 ± 21.8 | .601* |
| Hemoglobin F, % | 8.2 ± 7.6 | 4.1 ± 4.3 | .143* |

A total of 106 blood samples were obtained from 55 patients with homozygous HbSS (29 males and 26 females); the mean value was used for a subject tested more than once. Data are mean 6 standard deviation. P values that represent a statistically significant difference between group 1 and 2 are denoted with boldface.
*Nonparametric Mann-Whitney U test.
**Parametric 1-way analysis of variance (ANOVA).

Next, we examined associations between fetal hemoglobin (HbF) and RBC adhesion to ICAM-1 (FIG. 11A-B). We assessed RBC adhesion with respect to subjects' HbF levels, with a cutoff value of 8.6%, which was associated with improved life expectancy and a decrease in VOCs in natural history studies. We found that HbSS RBCs from subjects with higher HbF levels (n=18) had significantly lower adhesion to ICAM-1 compared with those with lower HbF levels (n=37) (FIG. 11C; 283±107 per fov vs 2127±649 per fov; mean adhesion±SEM, respectively; P=0.007). Moreover, 14 of 37 subjects with lower HbF levels (<8.6%) had RBC adhesion that was higher than the mean adhesion level, whereas only 1 of 19 subjects had HbF levels. 8.6%. Hydroxyurea (HU) treatment had a significant impact on HbF levels in our cohort (P, 0.001, x² test). Fourteen of 18 (77.8%) subjects with an HbF level. 8.6% were on HU compared with only 5 of 37 subjects (13.5%) with an HbF level, <8.6%. Of note, RBC adhesion did not correlate with subjects' hemoglobin A levels (i.e., prior transfusions; data not shown).

History of Intracardiac/Intrapulmonary Right-to-Left Shunts is Associated with Higher RBC Adhesion to ICAM-1

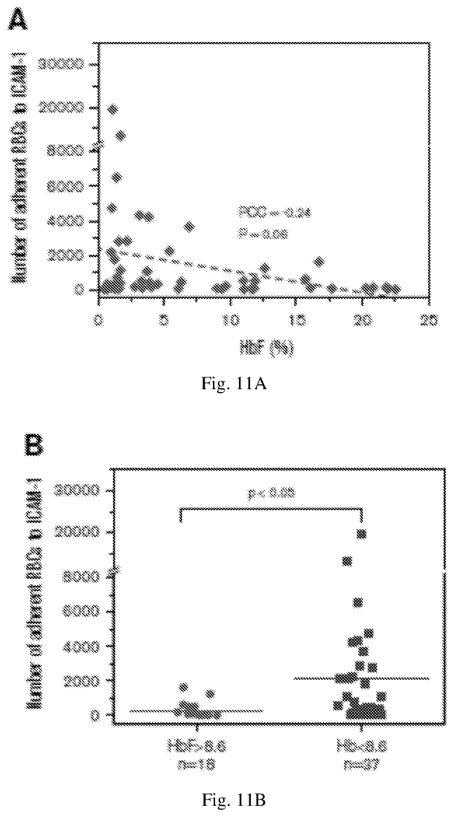

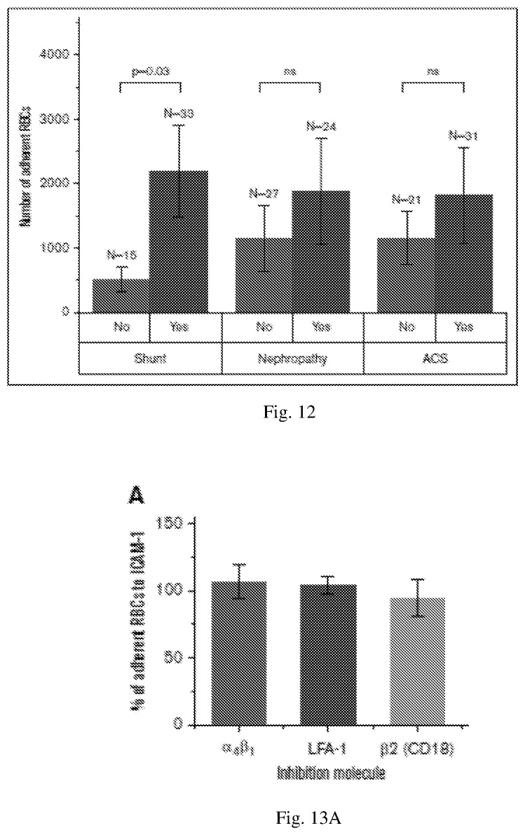

We next analyzed the adhesion data for select clinical comorbidities, including intracardiac or intrapulmonary shunts, concurrent nephropathy, and history of acute chest syndrome (ACS). Our results showed that people with HbSS and a history of right-to-left shunts had significantly higher RBC adhesion to ICAM-1 compared with people with HbSS and no history of right-to-left shunts (FIG. 12; 2184±717 per fov vs 515±738 per fov; mean adhesion±SEM, respectively; P=0.03). Although RBC adhesion was typically higher for subjects with a history of nephropathy or ACS compared with those without, we did not detect a statistically significant difference. These results suggest that higher RBC adhesion to ICAM-1 may be a surrogate biomarker of right-to-left intrapulmonary or intracardiac shunts in people with HbSS.

### Longitudinal Monitoring of RBC Adhesion to ICAM-1

We obtained multiple data points from 12 individuals with HbSS SCD at steady-state to monitor the variation in RBC adhesion and its association with clinical biomarkers (LDH, ARC, and WBC count) over time. Four subjects had very low adhesion levels, which did not change over time (<50 RBCs). Nineteen data points were obtained from 8 people: 6 subjects had RBC adhesion data from 2 time points, and the other 2 subjects had data from 3 and 4 time points. Between each time point, or interval, we determined whether subjects' RBC adhesion and clinical biomarkers changed or remained the same, with a total of 11 evaluable intervals (i.e., 1 interval for subjects with 2 time points and 2 and 3 intervals for subjects with 3 and 4 time points, respectively). We found that an increase or decrease in LDH levels and ARC was typically accompanied by an increase or decrease in RBC adhesion, whereas WBC counts did not seem to correlate with RBC adhesion. A change in LDH levels and ARC was reflected by a change in RBC adhesion for 8 of 11 (73%) intervals and 7 of 11 (64%) intervals, respectively. On the other hand, WBC counts reflected a change in RBC adhesion in only 3 of 11 intervals (27%). These results suggest that RBC adhesion may correlate with LDH and ARC over time. Nevertheless, it is still possible to detect changes in RBC adhesion, despite stable LDH levels and ARC, indicating that different factors could be at play with regard to RBC adhesion at different time points.

HbSS RBC Adhesion to ICAM-1 is Mediated by Fibrinogen, which Acts as a Bridging Molecule Between RBCs and Immobilized ICAM-1

To determine possible mediators of HbSS RBC adhesion to immobilized ICAM-1, we performed adhesion-inhibition experiments. First, we inhibited α₄β₁ integrin on the surface of RBCs to assess the adhesion of reticulocytes that were previously shown to adhere to fibronectin and VCAM-1 through α₄β₁. However, inhibition of α₄β₁ integrin did not result in reduced RBC adhesion (FIG. 13A). Next, we tested whether HbSS RBC adhesion to ICAM-1 is mediated by integrin-type receptors on the red blood cell membrane, considering the extensive characterization of the role of β₂-associated integrin in WBC-ICAM-1 interactions. Again, we found that neither the treatment of the blood samples with anti-LFA-1 antibodies nor incubation of the ICAM-1-immobilized microchannels with recombinant human b₂ protein prior to sample application had an inhibitory effect on the adhesion of HbSS RBCs to ICAM-1 (FIG. 13A). Therefore, we used fibrinogen to block the fibrinogen-binding domain of the immobilized ICAM-1 on the channel surface to prevent sample plasma fibrinogen from enhancing adhesive interactions during blood flow. Notably, treatment of microchannels with fibrinogen significantly decreased RBC adhesion in a concentration-dependent manner (FIG. 13B). It has been reported that plasma fibrinogen levels were in the range of 2 to 5.5 mg/mL for healthy subjects and 3 to 11.5 mg/mL for subjects with HbSS SCD. Therefore, we used 4 fibrinogen concentrations to simulate physiologic and pathophysiologic conditions: 1, 5, 10, and 20 mg/mL. Incubation of ICAM-1-immobilized microchannels with 1 mg/mL of fibrinogen did not inhibit RBC adhesion, whereas using a fibrinogen concentration of 5 mg/mL blocked adhesion by about 50% (FIG. 13B), suggesting that supranormal fibrinogen levels in HbSS blood samples may decrease RBC adhesion to ICAM-1. Because LMWH can bind to fibrinogen, we next treated whole blood samples with LMWH (1:1 volume to volume ratio) at concentrations of 0.1, 1, and 5 mg/mL, all of which significantly blocked RBC adhesion to ICAM-1 (FIG. 13C); on the other hand, preincubation of microfluidic channels with LMWH had no effect (data not shown). These findings suggest a mediating role for plasma fibrinogen in HbSS RBC adhesion to ICAM-1.

### HbSS RBCs Roll on ICAM-1 at Higher Shear Rates and Establish a Firm Attachment at Lower Shear Rates

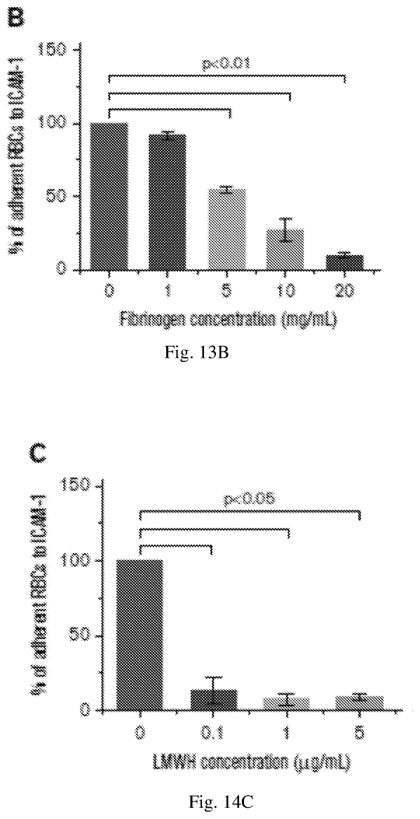

We observed that the total number of adherent RBCs decreased with increasing shear rate (FIG. 14A), whereas adherent RBCs persisted even after exposure to extreme shear rates (up to 5000 s⁻¹). We also observed a distinct motion of adherent RBCs, depending on flow shear rate. Some of the adherent RBCs exhibited rolling adhesion on ICAM-1, particularly at higher shear rates (FIG. 14B), and the percentages of rolling RBCs increased at shear rates of 3000, 4000, and 5000 s⁻¹ compared with those at 500, 1000, and 2000 s⁻¹ (FIG. 14C). Similarly, RBC rolling velocities increased at higher flow shear rates, with the maximum occurring at 5000 s⁻¹ (FIG. 14D), suggesting that the characteristic adhesion behavior shown herein may prevent physiological flow rates from removing persistently attached RBCs in the microvasculature.

Endothelial activation has been thought to have an important role in SCD pathology and VOC. There are several adhesion molecules that have been associated with an activated phenotype in SCD, such as ICAM-1, VCAM-1, E-selectin, and P-selectin. These adhesion molecules mediate the attachment and movement of blood cells to the endothelium and into peripheral tissue while increasing microvascular permeability. ICAM-1 expression has been shown to be induced by various inflammatory signaling pathways. Although the interactions between WBCs and ICAM-1, abundantly found on the activated endothelial lining, have been well characterized, the role of ICAM-1 in supporting HbSS RBC adhesion to ECs has never been identified. Here, our results demonstrated significant HbSS RBC adhesion to immobilized ICAM-1 presenting unique dynamics of motion, which were heterogeneous and subject-specific. Our results show that increased RBC adhesion to ICAM-1 correlates with hemolysis and a history of right-to-left shunts. Moreover, variations in RBC adhesion to ICAM-1 over time appear to correlate with LDH and ARC: an increasing or decreasing level of LDH or ARC corresponds to an increase or decrease in RBC adhesion. These observations are consistent with the “hyperhemolytic paradigm” that establishes that hemolysis in SCD increases endothelial dysfunction along with increased free plasma hemoglobin and NO biodeficiency and consumption.

HbF is one of the most studied genetic modulators of SCD because of its protective role in the disease. An increase in HbF levels correlates with improved survival and a milder disease phenotype, as evidenced by the lower frequency of VOCs and decreased mortality. Here, we found that HbF was inversely related to the adhesion of RBCs to ICAM-1. Interestingly, HbF levels seem to have a stronger effect than transfusions on decreasing RBC adhesion. Our data indicated that, even subjects with relatively lower HbS levels may exhibit enhanced RBC adhesion to ICAM-1 (data not shown). Because the majority of subjects with higher HbF levels were on HU therapy, we cannot rule out the potential direct impacts of HU on RBC adhesion, independent of an HbF-related mechanism. Nevertheless, a direct inhibitory effect of HbF on RBC adhesion was also noted in a cohort consisting of infants and young children with no HU therapy. These findings suggest that increased HbF can inhibit, at least in part, RBC adhesion in a HU-independent mechanism.

Although adhesion of Plasmodium falciparum-infected RBCs (iRBCs) to ICAM-1 has been well established, HbSS RBC adhesion to ICAM-1 has not yet been documented. It has been shown that P. falciparum erythrocyte membrane protein 1 is implicated in iRBC adhesion to ICAM-1 as the result of a binding site on the first domain of ICAM-1, which overlaps with the fibrinogen binding domain. iRBCs were shown to bind to ICAM-1 at a site that was distinct from LFA-1, MAC-1, and human rhinovirus. Further, blocking the fibrinogen binding site of ICAM-1 diminished iRBC adhesion to ICAM-1 in static and flow adhesion assays. Fibrinogen plays a vital role in the establishment of intercellular bridging between WBCs and ICAM-1, independently from LFA-1- and MAC-1-related pathways. We hypothesized that adhesion of HbSS RBCs to immobilized ICAM-1 in our microfluidic model was driven through a pathway involving plasma fibrinogen, because the HbSS RBC membrane lacks b2 integrins. Indeed, mixing the blood samples with an antibody against LFA-1 or incubating the ICAM-1-immobilized microchannels with β₂ integrin did not have any effect on RBC adhesion. Similarly, blocking the α₄β₁-dependent pathway did not have any effect on RBC adhesion, suggesting that little or no contribution was made by reticulocytes.

Our results reveal that HbSS RBC adhesion was significantly attenuated upon fibrinogen pretreatment of the microchannels immobilized with ICAM-1 in a concentration-dependent manner. These results indicate that HbSS RBCs do not directly interact with ICAM-1; rather, membrane-bound fibrinogen, which was characterized previously, triggers the adhesive connection by forming an intercellular bridge. To further support this, we incubated whole blood samples with varying concentrations of LMWH, which has been shown to bind to fibrinogen, prior to the flow adhesion experiments. Notably, LMWH pretreatment of blood samples, but not of the microchannels themselves, significantly blocked RBC adhesion to ICAM-1, even at very low doses (i.e., 0.80% inhibition at 0.1 mg/mL).

Although elevated plasma fibrinogen levels are linked to an increased risk for thrombosis, they may also interfere with ICAM-1-mediated RBC adhesion to decrease the frequency of VOC episodes. Upregulation of ICAM-1 through the vascular endothelium is triggered by various plasma cytokines and takes place at different levels, depending on the origin of the tissue. Therefore, a high fibrinogen level may alleviate cellular adhesion at sites where ICAM-1 is predominantly expressed while increasing the likelihood of thrombotic complications. Our results suggest therapeutic approaches targeting the fibrinogen binding domain of ICAM-1 may decrease ICAM-1-mediated HbSS RBC adhesion.

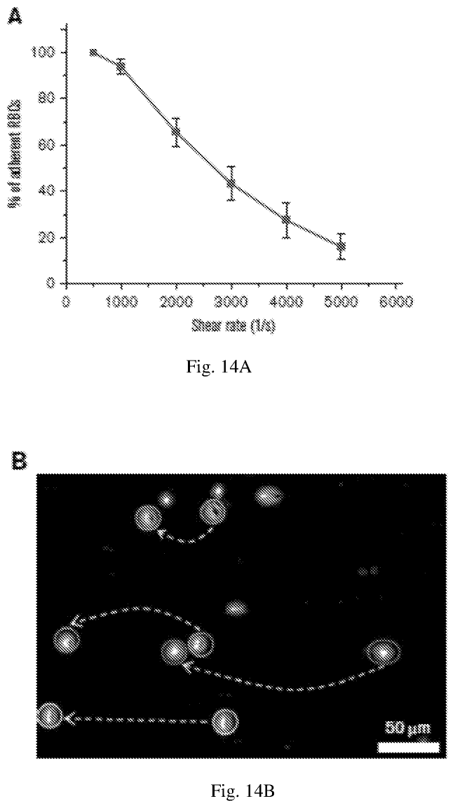

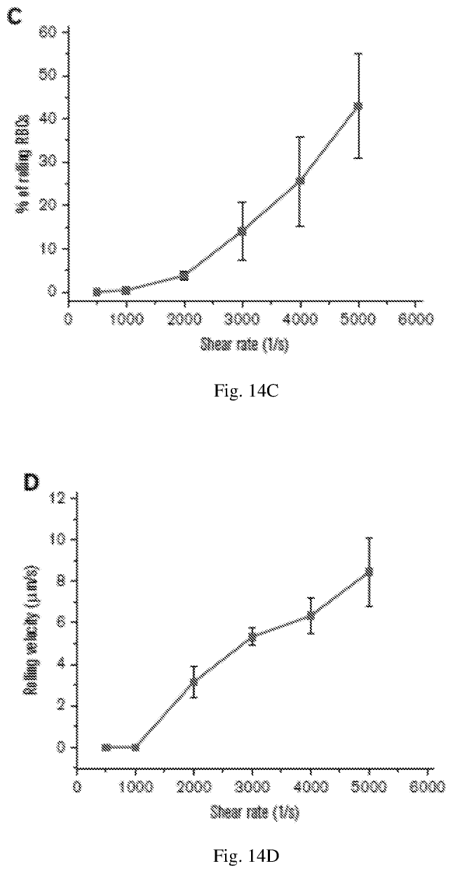

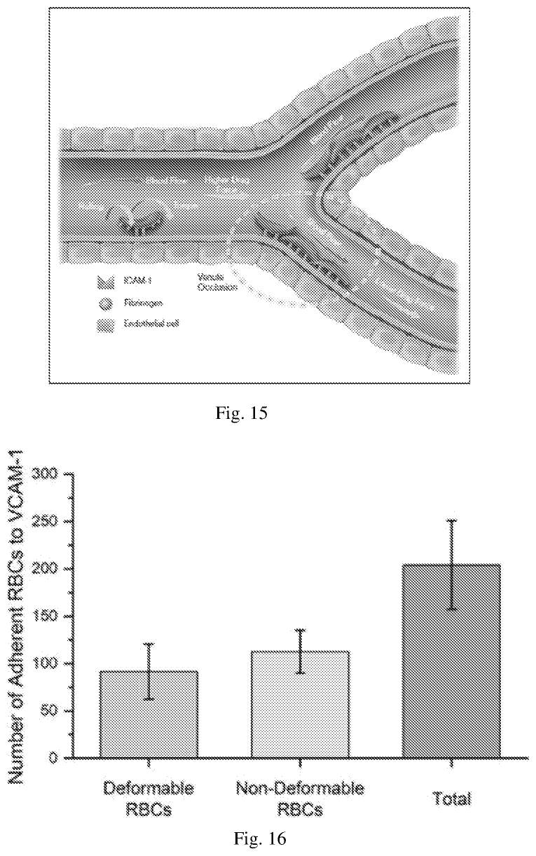

Furthermore, our results revealed that interactions analogous to leukocyte rolling on endothelium occur when HbSS RBCs bind to ICAM-1 at high shear rates. Similar rolling behavior of iRBCs interacting with ICAM-1 has also been documented. In the case of leukocytes, initial capture and rolling are established through receptors from the selectin family (e.g., E-selectin), after which firm attachment is accomplished via integrins LFA-1 and MAC-1. Meanwhile, in the case of P falciparum infection, adhesion of iRBCs mediated by ICAM-1 causes rolling and static adhesion over a wide range of shear rates. However, it was reported that ICAM-1 was not able to successfully immobilize iRBCs in the absence of CD36, which leads to the following question: Why does ICAM-1 act as a rolling receptor and a firm adhesion receptor? In accordance with previous discovery, we reported the rolling behavior of HbSS RBCs on immobilized ICAM-1, as well as that the ratio of rolling cells/firm adherent cells increased with increasing shear rate. Because we have shown that such HbSS RBC-ICAM-1 interaction is mediated by an intermediate molecule, fibrinogen, we speculate that the bridging of fibrinogen to ICAM-1 might have a cutoff stress threshold, such that the bond might be distorted under high shear and, thus, rupture under a critical stress load. It is also likely that rolling RBCs may have fewer adhesion receptors for fibrinogen on their membrane, preventing them from establishing a firm attachment to ICAM-1 under high shear stress. Because reticulocytes have more binding sites for fibrinogen, it is tempting to speculate that the majority of firmly attached RBCs are younger relative to the rolling RBCs. On the other hand, adhesion characteristics of HbSS RBCs onto ICAM-1, whereby firm adhesion dominates under low shear rates but rolling with increasing velocities occurs under high shear rates, may provide unique insights into the mechanism of the initiation of VOC-involved HbSS RBC-ICAM-1 interactions (FIG. 15). We postulate that, under inflammatory conditions in which endothelial ICAM-1 levels are upregulated, HbSS RBCs roll in capillaries, where the physiological shear rates are relatively higher, and firmly attach to the vasculature in postcapillary venules where shear rates are lower, thus contributing to increased resistance to blood flow and vaso-occlusion.

We used a physiologically relevant microfluidic platform to quantitatively evaluate adhesion of HbSS RBCs to immobilized ICAM-1. Our results pointed to a link between adhesion levels and high-grade hemolysis, as well as a history of right-to-left shunt development for subjects with HbSS SCD. Subjects with higher HbF levels had a milder adhesion profile. We further uncovered the role of plasma fibrinogen in mediating such HbSS RBC-ICAM-1 interactions and characterized the motion of adherent RBCs on ICAM-1, where they adhered firmly under low shear rates but rolled with increasing velocities under high shear rates.

### Example 4

This example describes a microfluidic device that includes a microchannel functionalized with human recombinant VCAM-1 protein for measuring RBC adhesion to VCAM-1 using whole blood cells from individuals with SCD. The VCAM-1 functionalized microchannel is coupled to a constant displacement pump that provides a physiologically relevant shear stress value of 1 dyne/cm² to flow the blood sample that is contained in a syringe. The microchannels are connected to the syringe by the inlet silicone tubing and mounted on the stage of an inverted microscope coupled with a charge-coupled device (CCD) camera to capture a scanned image of the microchannel surface (32 mm²) in a field of interest for quantification of RBC adhesion.

### Test Method

### RBC Adhesion Measurement

The assembled microfluidic channels are functionalized with VCAM-1, blocked with bovine serum albumin (BSA), and connected to a constant displacement pump. A 15 μl of blood sample is flowed across the microchannel at a constant flow rate and non-adherent cells are washed off by a buffer containing phosphate buffer saline (PBS) and 1% bovine serum albumin. The adherent RBCs are then quantified.

To characterize RBC adhesion to VCAM-1, 12 samples from 12 subjects with homozygous SCD (HbSS) were tested using VCAM-1 functionalized microfluidic channels. The range of total adherent RBCs (deformable+nondeformable) to VCAM-1 for the study population was between 19 and 546 with an average of 204 (FIG. 16). We observed two RBC subpopulations: deformable RBCs and non-deformable RBCs. Deformable RBCs had a characteristics morphology with a classic “donut shape” whereas non-deformable RBCs mainly exhibited an elliptical or sickle-looking morphology.

### Example 5

This example describes a microfluidic device that includes a microchannel functionalized with human endothelial cells cultured under physiologically relevant flow conditions. The cultured endothelial cells may be activated via a variety of stimuli, including heme, TNF-α, hydrogen peroxide, and thrombin. Before and/or after activation, whole blood samples and isolated blood components (e.g., red blood cells, white blood cells, or platelets) are flowed over the endothelial cells and allowed to establish adhesive interactions. Phenotypic characterization of quiescent or activated endothelial cells can also be performed separately. Abnormal blood cell adhesion to vascular bed is implicated in a multitude of cardiovascular and blood disorders as in sickle cell disease.

In this example, endothelial cells were functionalized to a microfluidic device that was formed using a lamination-based fabrication technique based on laser micro-machined parts that allows the construction of the device within only 5 minutes. Additionally, the large-scale design of this device affords a significantly greater surface area compared to currently available endothelialized microfluidic systems (i.e., 32 mm² vs 0.1 mm²). Having a large interrogation surface area significantly improves the ability to capture rare adhesive events for clinical samples with low adhesion potential. Furthermore, the relatively higher volume of the microchannel is likely to prevent any blockage or clogging when clinical samples with higher adhesion or aggregation rates are tested. Moreover, the use of gas impermeable components in this microchannel fabrication process allows the clinically relevant experiments to be carried out in a standard laboratory setting without the need for a specialized culture chamber, as gas exchange between the blood/media and the outside environment is limited. In contrast, the conventional lithography based platforms made of PDMS permit rapid exchange of gases, which necessitates maintaining the microchannel inside a culture chamber, impairing the clinical applicability of the system. Finally, having a gas-impermeable closed-loop flow system allows imaging on a heated-microscope plate, eliminating the need for expensive and complex on-stage incubators during image acquisition.

The microfluidic device described herein can be used to quantify blood cell adhesion to human endothelial cells that are cultured under physiologically relevant flow conditions in both normoxic and hypoxic conditions.

The microfluidic device includes a microfluidic platform pre-functionalized with fibronectin and seeded with human endothelial cells which can be analyzed either after a static culture phase or culture under flow phase. During the static culture phase, the microfluidic channels are placed in a controlled environment and allowed to incubate until the seeded cells have reached confluence. During the culture under flow phase, the microfluidic channels are connected to a reservoir that contains fresh cell culture medium suitable for the type of endothelial cells. The culture medium is circulated through the microfluidic system via a peristaltic pump at a flow rate corresponding to physiologic shear rates ranging from venous to arterial levels.

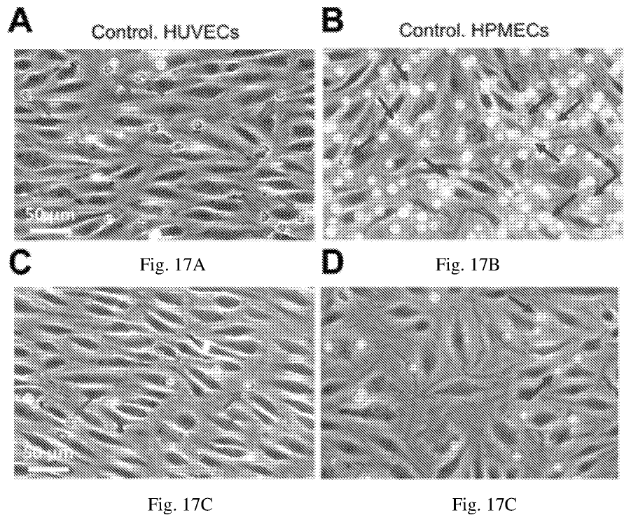

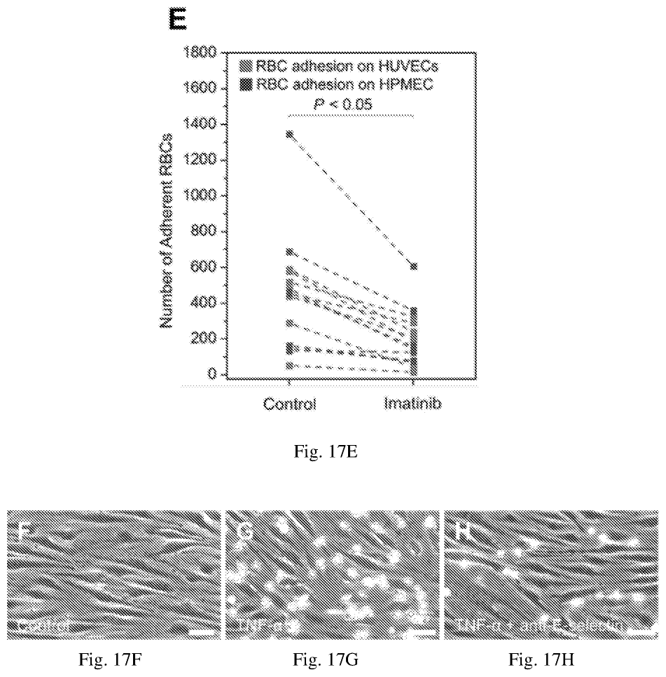

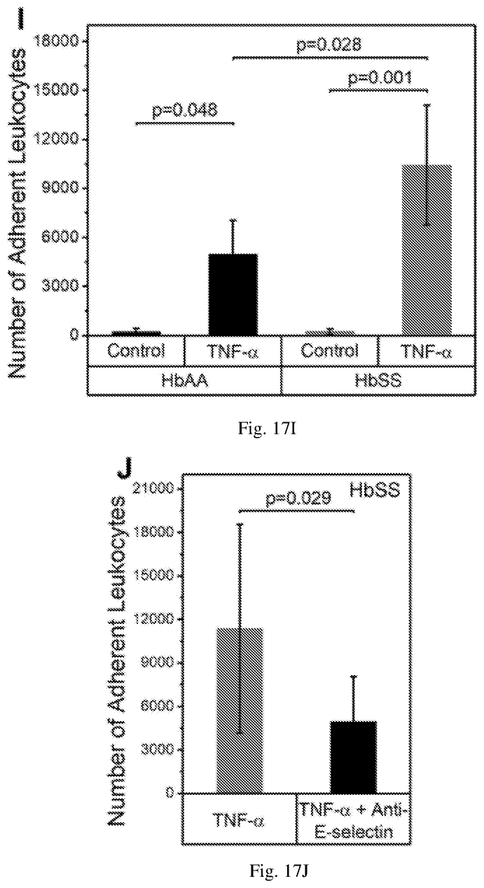

The microfluidic device in this example includes a microchannel, or series of microchannels, that contain adherent human endothelial cells on the microchannel surface. To accomplish cell culture under flow, the microchannels are coupled to a peristaltic pump and a reservoir that contains up to 15 mL of fresh cell culture medium in a polypropylene reservoir as illustrated in FIG. 17A. The microchannels are connected to each other through tygon tubing, and the inlet tubing is connected to the peristaltic pump via a combination of blunt needle, 0.2-micron sterile nylon syringe filter, luer connector, and silicone tubing. The purpose of the syringe filter in the system is to prevent any potential microbial contamination from reaching inside the microchannels. The peristaltic pump is connected to the reservoir via a combination of tygon tubing and luer connector. Finally, the circulating culture medium leaves the microfluidic system through a tygon outlet tubing and flows back into the reservoir through a combination of tygon tubing and stopcock. The reservoir is to remain in a controlled environment with a temperature of 37° C. and CO₂ concentration of 5% unless the culture medium is supplemented with a 15 mM of HEPES buffer solution, in which case a CO₂ concentration of 5% will not be necessary. Following a 24/48 hour of culture under flow, the endothelial cells will form a monolayer on the surface of the microchannels and be ready for subsequent experimental analyses.

### Test Method

### Device Endothelization

The assembled microchannels are rinsed serially with PBS, 100% ethanol, and GMBS following a 20-minute incubation. Thereafter, another washing step is performed using 100% ethanol and PBS before loading the microchannels with a fibronectin solution at a concentration of 0.2 mg/mL. Fibronectin-loaded microchannels are incubated at 37° C. for 1 hour for complete protein immobilization on the GMBS-functionalized surface. To prepare the endothelialized microchannels, human endothelial cells are seeded into fibronectin-coated microchannels at a density of 4×10⁶ cells/mL and incubated for 2 hours at 37° C. and 5% CO₂ to allow cell attachment and spreading, while replacing the culture medium in the microchannels every hour.

### Endothelial Cell Activation

Once the endothelial cells have reached confluence, in either static or flow conditions, the microchannels were rinsed with culture medium that does not contain serum and incubated for 1 hour. The activating agents (e.g., heme or TNF-α) are then injected into the microchannels in a serum-free medium and incubated for a pre-determined period for activation to take place. Before using the activated endothelial cells, the microfluidic channels are rinsed for at least 2 times with serum-free medium.

### Blood Cell Adhesion Measurements in Normoxia

When using blood samples collected in Ethylenediaminetetraacetic acid (EDTA) containing vacutainers, the blood samples are first centrifuged and washed three times with PBS to isolate blood cells from plasma. When in contact with adherent endothelial cells, EDTA will induce cell detachment by inhibiting Ca²⁺ ions that are necessary for cell adhesion through integrins. Isolated and washed blood cells are then re-suspended in fresh serum-free culture medium supplemented with 10 mM HEPES buffer solution at a fixed hematocrit. The sample is then perfused into the microchannels at a physiological or pathophysiological shear stress for a fixed amount of time. Non-adherent cells are rinsed away by injecting fresh serum-free culture medium supplemented with 10 mM HEPES buffer solution into the microchannels at the same shear stress.

### Blood Cell Adhesion Measurements in Hypoxia

To conduct experiments in hypoxia, the microfluidic platform is integrated with a micro gas-exchanger system that is used to impose hypoxia on the sample before it enters into the microchannel. The system consists of a medical grade gas-permeable silicone tubing placed inside an impermeable tubing, which allows gas exchange between the blood flow and 5% CO₂ and 95% N₂-controlled gas through the permeable tubing wall inside the impermeable tubing by diffusion, resulting in an SpO₂ of 83%.

The effect of imatinib, a tyrosine kinase inhibitor, on the adhesion of flowing sickle RBCs to heme-activated endothelial cells under hypoxia was assessed. As seen in representative images of adherent RBCs in FIG. 17, untreated RBCs (FIG. 17A-B) are more adherent to heme-activated human umbilical vein endothelial cells (HUVECs) and human pulmonary microvascular endothelial cells (HPMECs) under hypoxia than imatinib-treated sickle RBCs (FIG. 17C-D). These data suggest that imatinib can reduce vaso-occlusive events by suppressing adhesion of sickle RBCs to activated endothelial cells.

Next, white blood cell (leukocyte) adhesion to HPMECs using blood samples from healthy and SCD subjects. Representative images of adherent leukocytes from HbSS samples exposed to control, TNF-α activated, or activated and anti-E-selectin treated HPMECs in microchannels are shown (FIG. 17F-H). TNF-α activation of HPMECs led to significantly increased number of adherent leukocytes compared to control in the HbAA subjects (FIG. 17I). Significantly greater number of adherent leukocytes on TNF-α activated HPMECs compared to control was also found in the HbSS subjects (FIG. 17I). Further, the number of adherent leukocytes was significantly greater in the HbSS subjects compared to the HbAA subjects (FIG. 17I). Importantly, these results suggest that E-selectin is an important target receptor on endothelium for blocking pulmonary vaso-occlusive events in SCD, as an adhesion blocking anti-tin antibody significantly inhibited leukocyte adhesion to TNF-α activated HPMECs in the HbSS subjects (FIG. 17J).

### Example 6

In this example, we describe a microfluidic system integrated with a micro particle image velocimetry (PIV) technique for integrated in vitro assessment of whole blood rheology and red blood cell adhesion in a clinically useful manner

Although the distinct effects of plasma viscosity, impaired RBC deformability, and elevated plasma protein levels on WBV have been very well established in literature, a more comprehensive approach that takes into account all these factors in a patient-specific fashion is needed to better understand the role of WBV in the pathophysiology of SCD. Here, we quantitate WBV using pre-processing free whole blood samples, without adopting the “HCT-matching” technique, allowing patient-specific as well as clinically relevant WBV measurements. This method is unique in that we simultaneously report WBV and RBC adhesion characteristics, under normoxic and hypoxic conditions, of individuals with SCD and associations with clinical variables. This integrated approach is pivotal since both WBV and RBC adhesion are dictated by a myriad of parameters in SCD. This platform has great potential to provide a multifaceted platform for a more comprehensive evaluation of new and emerging therapeutic interventions in SCD, including treatments designed to change red cell properties, including stem cell and gene-based curative therapies.

### Materials and Methods

### Blood Collection

Whole blood samples from de-identified adult (≥18) healthy donors and SCD subjects seen in the Adult Sickle Cell Clinic at University Hospitals Cleveland Medical Center (UHCMC, Cleveland Ohio) were collected in EDTA (Ethylenediaminetetraacetic acid) containing vacutainers based on an Institutional Review Board (IRB) approved protocol. All collected samples were stored at 4° C., and the experiments were conducted within 24 hours of venipuncture. Clinical information, medical treatments and previous comorbidities, were obtained, including total hemoglobin level, red blood cell (RBC) count, white blood cell (WBC) count, platelet numbers, lactate dehydrogenase (LDH) levels, mean corpuscular volume (MCV), and hemoglobin phenotype (via high-performance liquid chromatography (HPLC) with the Bio-Rad Variant II Instrument (Bio-Rad, Montreal, QC, Canada) at the Core Laboratory of UHCMC). Only subjects infected with HIV or hepatitis C were ineligible for this study.

### Fabrication of Microfluidic Platforms

A double sided adhesive (DSA) polyester film was placed in between a top polymethyl methacrylate (PMMA) cover and a bottom glass microscope slide pre-coated with 3-Aminopropyl Triethoxysilane (APTES, Gold Seal Electron Microscopy Sciences, Hatfield, Pa.). The DSA film and PMMA top cover were laser micro-machined to define the microchannel walls as well as inlet and outlet ports. The assembled devices consisted of 3 identical microchannels with dimensions of 4 mm×25 mm×0.05 mm (width×length×height). The height of the microchannels was chosen to mimic the size scale of post-capillary venules as it has been shown that this part of the microvasculature plays a critical role in the initiation and progression of VOE events.

### Micro Particle Image Velocimetry Setup for Normoxic Viscosity Measurements

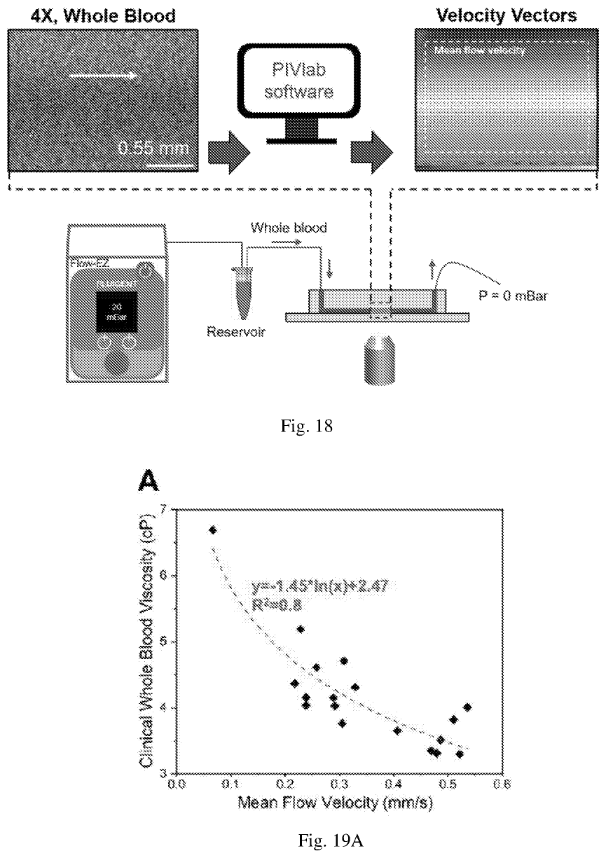

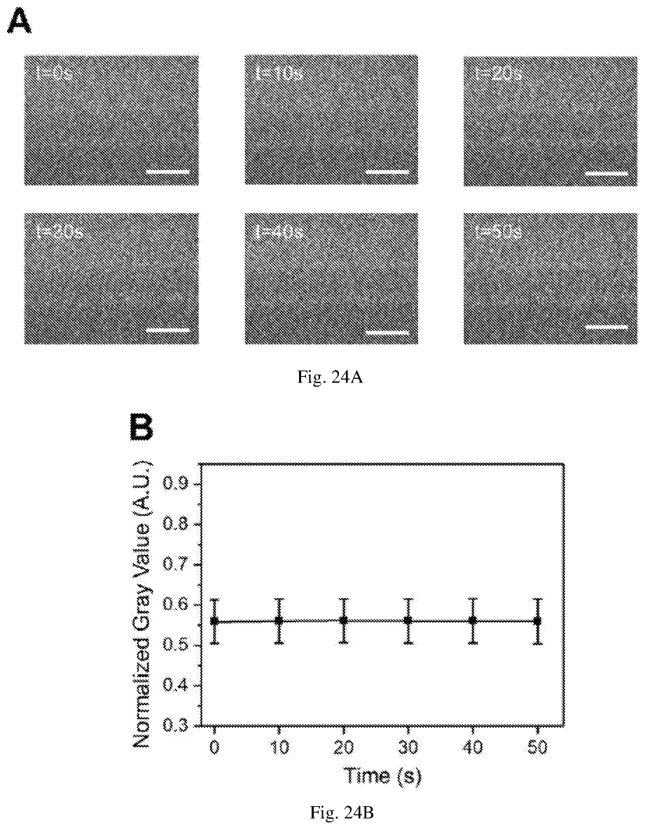

The assembled microfluidic channels were rinsed with 100% ethanol and PBS, and were equipped with silicon tubings that were fixed with epoxy at the inlet and outlet connection ports. A Flow EZ™ microfluidic flow control system (Fluigent) was used to regulate the flow pressure in the microfluidic channels. The microchannels were connected to the input well by the inlet silicone tubing and male luer connectors, and were mounted on the stage of an inverted microscope (Olympus IX83) coupled with a charge-coupled device (CCD) microscopy camera (EXi Blue EXI-BLU-R-F-M-14-C) to obtain high-resolution videos of the blood flow, following the experimental setting in FIG. 18. For each measurement, 500 μL whole blood sample was loaded in the input well and perfused at a constant pressure of 20 mBar, and two videos of 500 frames were taken at 10 frames per second in two different locations along the channel length. Data acquisition began 1-minute after the initiation of flow to allow it to reach a steady state. To ensure that no significant RBC settling occurred during the experiments, we quantified the grayscale intensity of the images acquired for 50 seconds following the initial 1-minute waiting period. The image grayscale intensity has been shown to correlate with sample HCT. FIG. 24 illustrates that the grayscale intensity of the images recorded sequentially for 50 seconds (representing the entire experiment duration) remained nearly unchanged, indicating there was no significant RBC settling in the reservoir.

### Micro Particle Image Velocimetry Setup for Hypoxic Viscosity Measurements

To achieve a physiologically relevant oxygen tension in flowing blood, we coupled a micro-gas exchanger at the inlet of the microchannel. Briefly, a medical grade gas-permeable silicone tubing (Dow Corning) was placed inside an impermeable tubing (Cole-Parmer) to allow gas exchange between the blood flow and 5% CO₂ and 95% N₂-controlled gas through the permeable tubing wall inside the impermeable tubing by diffusion, resulting in an SpO₂ of 83% when blood flow reached the inlet of the microchannel. Similar to normoxic viscosity measurement, 500 μL whole blood sample was loaded and perfused at 20 mBar, and a video of 600 frames was acquired in the middle of the microchannel under normoxic condition after steady-state flow condition. Thereafter, the blood was still allowed to perfuse while the controlled gas was turned on, and a video of 6000 frames was started at the same time in the same field of view.

### Quantification of Mean Flow Velocities

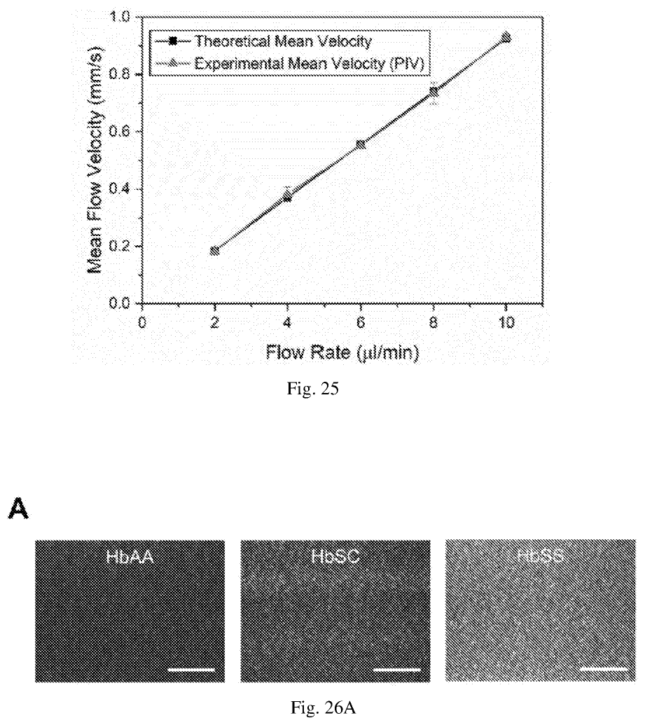

Frames of the recorded videos were extracted using Adobe Photoshop CS5. A total number of 250 pairs of images (500 frames) were cross correlated to obtain the velocity vector maps using a customized Matlab Code (PIVLab). The time interval between each successive image was set to 100 ms, which was the frame per second rate of the CCD camera. The cross correlation procedure was carried out within 2 passes, in which the size of the interrogation areas was 256×256 pixel with 50% overlap during the first pass, and the information collected in this pass was utilized for the calculations during the second pass within smaller interrogation areas (128×128 pixel). The velocity vector maps (250 in total) were then averaged to obtain an average velocity vector map. 20% of the interrogation area was cropped near the edges, and the rest of the mean vector map was again averaged to calculate the average flow velocity. The outliers were removed for each velocity vector map (250 in total) using the built-in function included in the Matlab code. Because we used a volume-illumination method, the average velocity vectors contained information throughout the entire microchannel depth. To confirm that this velocity would correspond to mean flow velocity, we repeated these experiments using a constant displacement syringe pump and compared our PIV calculations against the theoretical mean flow velocity, which is given by the formula below:

$$\overline{V} = \frac{Q}{A} \qquad ( 1 )$$

where v̅ is the mean velocity along the microchannel depth, Q is the volumetric flow rate, and A is the cross-sectional area of the microchannel. As shown in FIG. 25, our results indicate that the mean velocity obtained by the PIV experiments through the entire fluid volume actually represented the mean flow velocity in the microchannel.

### Clinical Whole Blood Viscosity Measurements

Whole blood samples from 19 subjects were sent to the clinic in University Hospitals at Cleveland Medical Center (UHCMC) in Cleveland, Ohio for standard reference viscosity measurements via a piston-style viscometer (Cambridge Viscosity, Boston, Mass.). The clinical viscosity measurements were conducted using whole blood samples upon request without any dilution or pre-processing.

### Microfluidic Channel Functionalization for RBC Adhesion Assays

The microfluidic channels were rinsed with 30 μL of PBS and ethanol after assembly. Next, 20 μL of cross-linker agent N-g-Maleimidobutyryloxy succinimide ester (GMBS, 0.28 mg/mL in ethanol) was injected into the channels twice and incubated for 15 min at room temperature, which was followed by 30 μL of ethanol and PBS washing. Thereafter, 20 μL of laminin (LN) solution (0.1 mg/mL) was injected into the channels and incubated for 1.5 hour at room temperature. The surface was then passivated with 30 μL of 2% bovine serum albumin (BSA) solution and overnight incubation at 4° C. Before the RBC flow adhesion assay, microchannels were rinsed with PBS.

### RBC Adhesion Assays Under Normoxic Condition

RBC adhesion assays were performed in separate microfluidic channels that were functionalized with a subendothelium protein, LN. In contrast to the viscosity experiments, a constant displacement pump was utilized here to provide a constant shear stress throughout the microchannels. The assembled and functionalized microfluidic devices were attached with an inlet tubing and placed on a motorized microscope stage (Olympus IX83). Undiluted whole blood samples were loaded into 1-mL syringes and a total volume of 15 μl of blood was injected into the microchannels at a constant shear stress of 1 dyne/cm² using a syringe pump (New Era NE-300, Farmingdale, N.Y.), followed by a wash step (1× PBS, 1% BSA, 0.09% Sodium Azide) at a shear stress of 1 dyne/cm², during which non-adherent cells were removed from the microchannel Thereafter, a 32 mm×32 mm interrogation area was scanned at 20× via the Olympus Cell Sense live imaging software, and the total number of adherent RBCs was manually quantified using Adobe Photoshop CS5 (San Jose, Calif., USA).

### RBC Adhesion Assays Under Hypoxic Conditions

Whole blood samples were perfused into the LN-functionalized microchannels with the micro-gas exchanger at 1 dyne/cm², which was followed by same washing, imaging, and quantifying as described above.

### Statistical Analysis

The statistical analyses were performed using Minitab 19 (Minitab Inc., State College, Pa.). Statistical comparison between two groups was conducted using the Student's t-test or paired t-test for non-paired and paired data respectively. To compare three or more groups, we performed Kruskal-Wallis test with Dunn's multiple comparison. A p-value below 0.05 was considered to indicate statistical significance. The data is reported as mean±standard deviation (SD).

### Results

### Mean Flow Velocity as a Surrogate for WBV

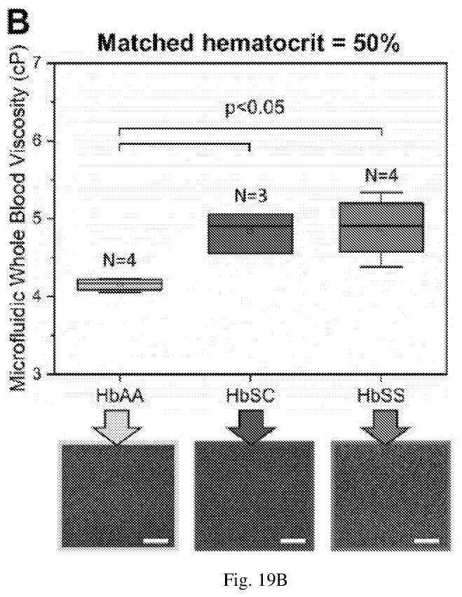

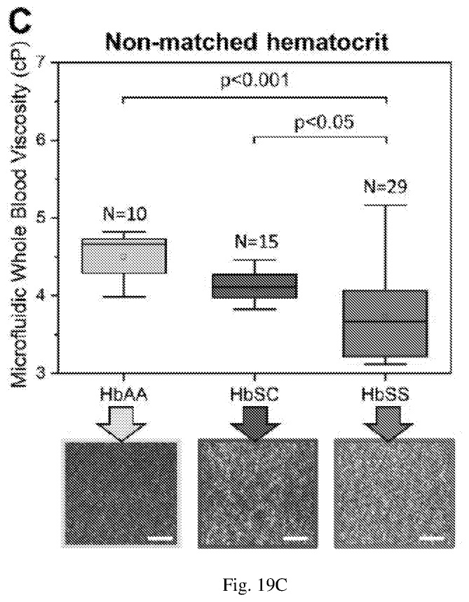

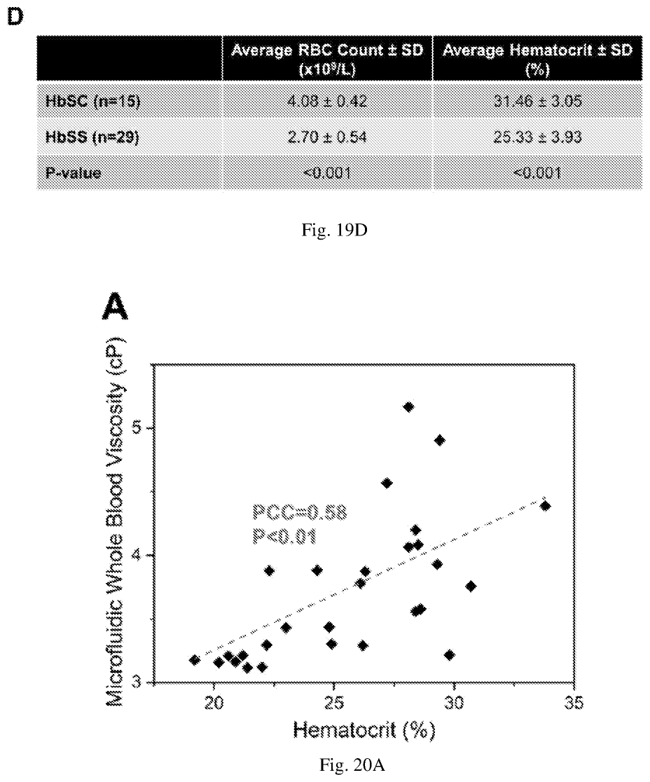

Here, we used mean flow velocities obtained via micro-PIV as a surrogate for whole blood viscosity. To correlate the measured flow velocities with the sample viscosity, we first obtained the viscosity of 20 blood samples that were measured via a commercially available piston-style viscometer (Cambridge Viscosity, Boston, Mass.). FIG. 19A shows that there existed an inverse logarithmic relationship between the sample viscosity and mean flow velocity in the microchannel at a constant pressure of 20 mbar, which was within the physiological range. Thus, we utilized the logarithmic equation to infer the microfluidic whole blood viscosity of the sample from the mean flow velocity determined by micro PIV, which will be referred as whole blood viscosity henceforth. Adhering to this methodology, we first quantified the viscosity of blood samples from subjects with no hemoglobinopathies (HbAA) and with homozygous sickle cell disease (HbSS) by utilizing the “HCT-matching” technique, in which the blood samples were initially centrifuged to separate RBCs from whole blood. Then, the isolated RBCs were mixed with plasma obtained from the same sample at a ratio of 1:1 yielding a fixed HCT of 50%. During this procedure, we strictly adhered to the hemorheological and laboratory techniques guidelines published in 2009. This approach allowed us to analyze the effect of RBCs and plasma on determining whole blood viscosity of a sample by eliminating any possible contribution from sample-to-sample HCT variation.

### Effect of Hematocrit Matching on WBV

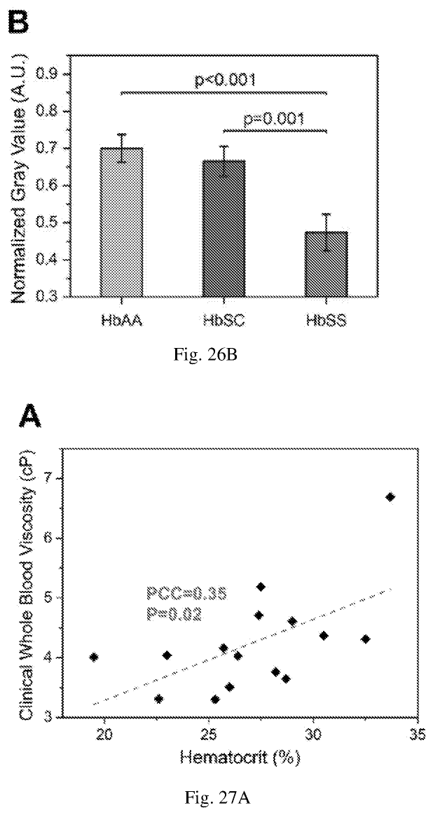

In line with previous findings, our results showed that viscosity of HCT-matched HbSS samples was significantly greater compared to HbAA samples (FIG. 19B, 4.89±0.41 cP vs 4.15±0.07 cP, p<0.05, Mann-Whiney U-test) likely due to abnormal RBCs and pro-inflammatory plasma from subjects with HbSS. However, it is well known that subjects with SCD suffer from chronic anemia and have much lower HCT values compared to people with HbAA, which may have a significant impact on their whole blood viscosity. Therefore, we next tested the viscosity of blood samples from subjects with HbAA, HbSC, and HbSS using undiluted and non-preprocessed whole blood. As shown in FIG. 19C, the mean viscosity level of HbAA samples was highest among, then HbSC, while HbSS samples displayed the lowest mean WBV, in contrast to the HCT-matched results (FIG. 19B). We observed a significant heterogeneity within the HbSS group, in which the difference between the lowest and highest data points was almost twofold. Of note, HbAA samples typically had lower grayscale intensities when imaged under a phase-contrast microscope compared to HbSC and HbSS samples, which is indicative of HCT (FIG. 26).

Measured WBV is Heterogeneous and Correlates with Hematological Parameters

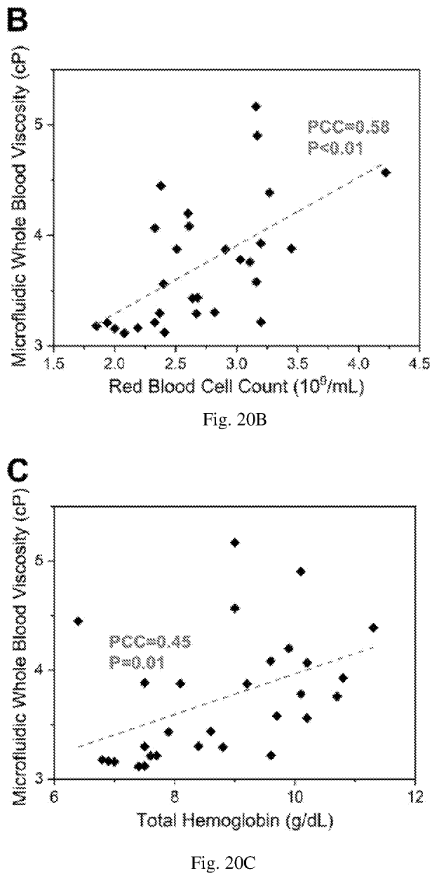

Viscosity of whole blood is determined by a number of factors including HCT (the ratio of RBC volume to whole blood volume). Our results show that both HCT and total RBC count associate with WBV, although the relationships were not entirely linear, demonstrating the likelihood of other possible contributors to WBV (FIG. 20A, HCT: PCC=0.58, p<0.01; FIG. 20B, RBC count: PCC=0.58, p<0.01, one-way ANOVA).

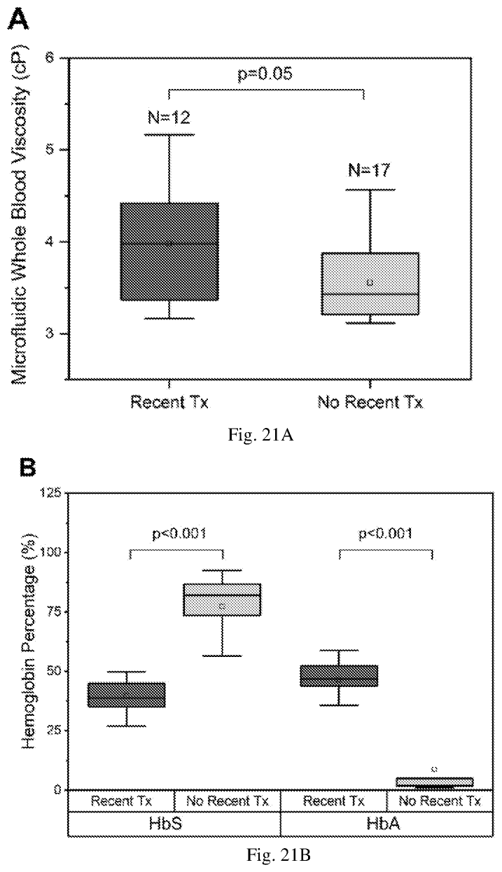

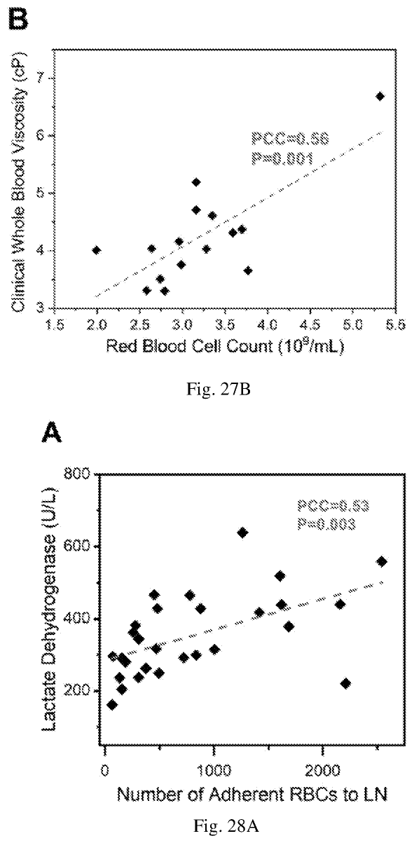

Further, subjects with a lower WBV tended to have significantly higher total hemoglobin levels (FIG. 20C, PCC=0.45, p=001, one-way ANOVA) although all the subjects in the study population had lower than normal HCT levels. These findings collectively suggest that our microfluidic approach accounts for rheological differences made by a number of determinants of WBV. Similarly, clinical measurements of WBV in people with homozygous or heterozygous SCD, using the piston-style viscometer, also displayed a significant association with subjects' RBC counts and HCT levels, albeit also not strongly linearly (FIG. 27, HCT: PCC=0.35, p=0.02; RBC: PCC=0.56, p=0.001, one-way ANOVA). These results confirm that our microfluidic assay provides similar results compared to a commercial viscometer with regards to the dependence of whole blood viscosity on sample HCT and total RBC count. No other significant associations between WBV and clinical variables, including lactate dehydrogenase (LDH), absolute reticulocyte counts, mean corpuscular volume, HbS levels, and fetal hemoglobin (HbF) levels, were detected in this modest-sized sample. However, subjects with a recent transfusion history (<3 months) had a significantly higher WBV compared to those with no recent transfusion (FIG. 21A, 4.01±0.7 cP vs 3.56±0.4 cP, p<0.05, Student's t-test). The subject population with a recent transfusion had significantly lower HbS levels (FIG. 21B, 39.7±6.5% vs 77.1±13.3%, p<0.001, Student's t-test). No association was observed between WBV and hydroxyurea treatment (data not shown).

WBV Correlates Inversely with RBC Adhesion

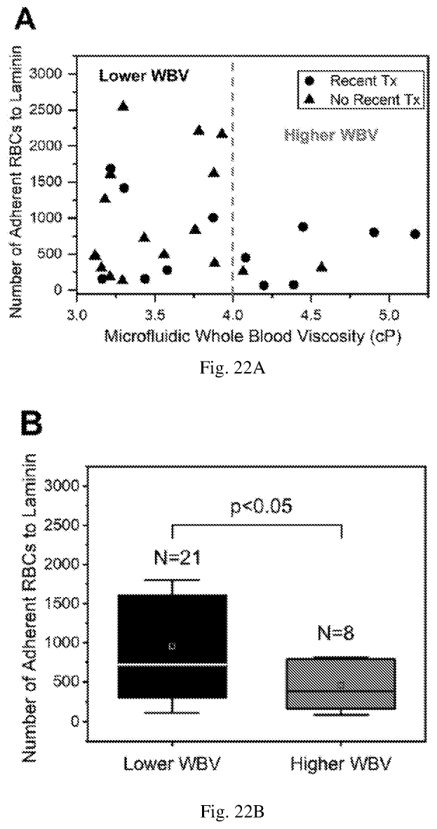

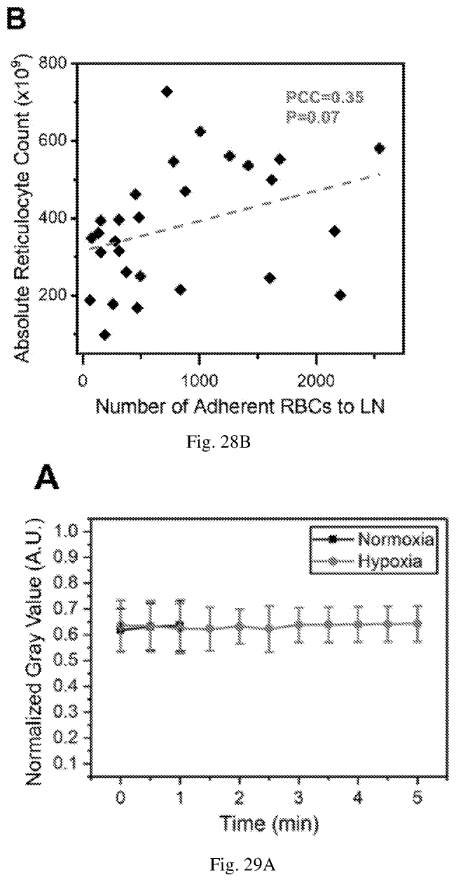

We and others have previously described an association between disease severity and RBC adhesion in SCD. Since our current results suggest a significantly heterogeneous WBV profile among patients with HbSS, we next sought to determine whether there was an association between RBC adhesion to the sub-endothelial protein Laminin (LN) and WBV. Ln has been repeatedly shown to mediate sickle RBC (HbSS RBC) adhesion through the RBC membrane receptor BCAM-Lu. Therefore, it constitutes a physiologically relevant substrate in assessing RBC adhesion in vitro. The adhesion experiments and WBV measurements were conducted in parallel using separate microfluidic channels. Because the lowest limit of WBV from tested HbAA samples was approximately 4 cP, we segregated the SCD study population into two groups: a subnormal (lower) WBV group (<4 cP, N=8) and normal (higher) WBV (>4 cP, N=21) as shown in FIG. 22A. Notably, subjects with a lower WBV displayed significantly greater RBC adhesion to LN compared to those with a higher WBV (FIG. 22B, 957±767 vs 452±331, p<0.05, Student's t-test). Moreover, RBC adhesion to LN strongly associated with subject LDH levels, and borderline so for absolute reticulocyte count (FIG. 28A, p<0.05, PCC=0.53; FIG. 28B, p=0.07, PCC=0.35, respectively).

### Hypoxia-Induced WBV Alteration is Subject-Specific and May Influence RBC Adhesion

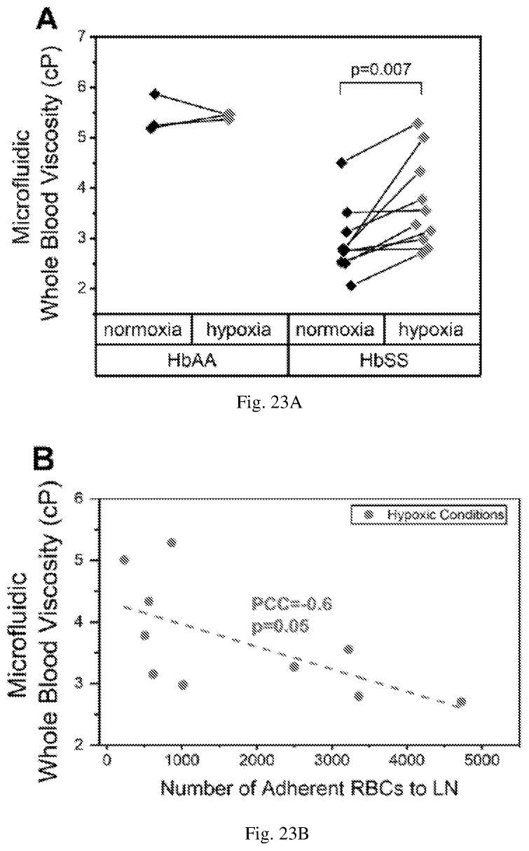

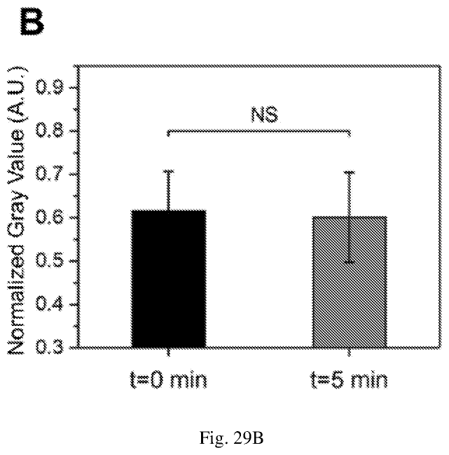

Polymerization of sickle hemoglobin (HbS) in hypoxia leads to abnormal RBC biophysical properties such as increased adhesiveness and impaired deformability. Therefore, a hypoxic environment may increase sickle WBV in an individual patient, due to the impact of RBC deformability. On the other hand, the ratio of HbS to normal hemoglobin (HbA) may be heterogeneous among individuals with SCD, who are often transfused. Finally, contribution of RBC deformability to WBV may be significantly altered depending on the HCT level. We integrated a micro gas exchanger to our microfluidic system, as we have previously described, in order to probe the change in WBV as well as RBC adhesion to LN deriving from the change from normoxic to hypoxic conditions. Because RBC settling in the reservoir could affect the measured viscosities, we quantified grayscale intensity changes of the images that were acquired for 5 minutes following flow initiation. As illustrated in FIG. 29, there was no significant change of RBC settling during the entire experimental setup, reflected by the absence of a grayscale intensity change Of note, the grayscale intensities in FIG. 29 do not represent flow velocity, and we did observe a significant reduction in mean flow velocity under hypoxia as shown in FIG. 23. The hypoxic viscosity results showed that WBV of control blood samples (HbAA, N=3) remained relatively unchanged, while individual HbSS samples (N=10) became more viscous under hypoxia as shown in FIG. 23A (p=0.007, Student's t-test). Interestingly, we observed an inverse relationship between hypoxic WBV and RBC adhesion to LN in hypoxia, similar to that seen under normoxic conditions (FIG. 23B, PCC=−0.6, p=0.03, one-way ANOVA). In other words, RBCs from samples with a lower hypoxic WBV had an increased propensity to adhere to LN under hypoxic conditions.

In this example, we quantified the viscosity of preprocess-free whole SCD blood samples from a clinically-diverse patient population. Our findings revealed that sample HCT played a pivotal role in determining whole blood viscosity. SCD samples were significantly more viscous compared to HbAA samples when HCT levels were matched, but the viscosity of SCD samples was heterogeneous and much lower compared to the HbAA group when pre-processing free whole blood was evaluated. A lower-than-normal WBV can lead to a lower endothelial shear stress in people without SCD, which has been linked to endothelial activation and cardiovascular disorders. In this example, we speculate that a significantly lower WBV contributes to the vascular dysfunction and related endothelial pathophysiology commonly seen in SCD. In addition, the microfluidic platform described in this study holds promise as a highly translatable system that could be utilized for simultaneous measurement of WBV and RBC adhesion for emerging targeted as well as curative therapies. For instance, WBV and RBC adhesion levels can be assessed before and after therapeutic interventions targeted at HCT augmentation, adhesion mitigation, and/or before and after a curative therapies, in order to assess changes in blood and RBCs with therapy.

Our results demonstrated that subjects with HbSS SCD had heterogeneous WBV profiles, with normal or subnormal WBVs compared to controls (HbAA), which may result in distinct pathophysiological consequences. A subnormal WBV depresses endothelial shear stress, which normally maintains endothelial health. A lower endothelial shear stress, has been established as a pro-inflammatory stimulus and associated with a risk for initiation and progression of coronary atherosclerosis. Therefore, we postulate that chronic sub-normal WBVs, due to anemia, may impose additional burdens to cardiovascular health and disease, particularly for people with HbSS SCD who already suffer from a high degree of micro and macro-vascular complications. On the other hand, an acute rise in WBV could mean a lower mean blood flow velocity, as seen in our microfluidic system under hypoxia, promoting RBC sickling, vaso-occlusion, and local ischemia due to increased RBC passage time through the microvasculature. This could increase local hypoxia, leading to a further rise in WBV and increased cellular interactions between RBCs and endothelial cells, slowing down the blood flow and so on.

Hypoxia is a strong modulator of whole blood rheology, particularly in SCD, as the biophysical properties of HbSS RBCs significantly change under low oxygen conditions, which in turn alters both WBV and cellular adhesion. Accordingly, we integrated a micro gas-exchanger, through which the hemoglobin saturation (SpO₂) was reduced to 83%, in to our microfluidic platform. We found no meaningful change in WBV between normoxic and hypoxic conditions using HbAA samples, but HbSS samples became significantly more viscous in hypoxia. Strikingly, hypoxia enhanced RBC adhesion, seen here and in earlier experiments, may acutely block the microvasculature and lead to locally elevated viscosity, which may be deleterious to the microvasculature in a way that higher (‘normal’) WBV is unlikely to be chronically.

In this example, we integrated analyses of WBV and, in a separate platform, RBC adhesion. We found an inverse association between RBC adhesion and WBV in people with HbSS. A lower WBV strongly associated with a lower HCT. Similarly, LDH levels and absolute reticulocyte counts were significantly higher in subjects with a higher RBC adhesion profile. Based on these findings, we speculate that a hemolytic environment associates with RBC adhesion and also leads to a lower WBV due to reduced HCT, which explains the inverse relationship between RBC adhesion and WBV. Further, a hemolytic environment has been reported to damage the endothelium, while a lower WBV and thus lower endothelial shear stress may mediate adhesive interactions between endothelial cells and blood cells in vivo, since cellular adhesion is strongly governed by applied shear force. Our integrated viscosity and adhesion analysis highlight a range of deleterious effects on the microvasculature that may arise in a patient with HbSS and hemolysis, from the cumulative effects of heme toxicity, abnormal cellular adhesion, and the impact of a low HCT and subnormal WBV on endothelial health. Further, acute, hypoxia-induced increases in viscosity and in RBC adhesion may together have a deleterious impact on the local environment of the microvasculature. Patients with low WBV and high cellular adhesion may represent an especially at-risk subpopulation.

From the above description of the invention, those skilled in the art will perceive improvements, changes and modifications. Such improvements, changes, and modifications are within the skill of the art and are intended to be covered by the appended claims. All patents and publications identified herein are incorporated by reference in their entirety.

1. A microfluidic system for measuring cell adhesion, the system comprising:
a gas impermeable housing including at least one microchannel defining at least one cell adhesion region, the at least one cell adhesion region being provided with at least one capturing agent that adheres a cell of interest to a surface of the at least one microchannel when a fluid sample containing cells is passed through the at least one microchannel, wherein at least the capturing agent includes at least one of E-Selectin, P-Selectin, intracellular adhesion molecule 1 (ICAM-1), vascular cellular adhesion molecule 1 (VCAM-1) or endothelial cells functionalized to the surface of the microchannel; and
an imaging system for measuring the adherence of cells of interest adhered by the at least one capturing agent to the surface of the at least one microchannel when the fluid sample is passed therethrough.

2. The system of claim 1, the at least one microchannel comprising multiple microchannels, the microchannels being fluidly isolated from each other.

3. The system of claim 1, the fluid comprising blood and the cells of interest being red blood cells.

4. The system of claim 3 further comprising a micro-gas exchanger for controlling the oxygen content of the blood prior to delivering the blood to the at least one microchannel.

5. The system of claim 4, the micro-gas exchanger providing hypoxic blood to the at least one microchannel.

6. The system of claim 1, the at least one microchannel having a width that continuously changes in a direction of fluid flow therethrough.

7. The system of claim 6, the microchannel having a convergent and divergent cross-sectional area along the direction of flow.

8. The system of claim 6, the shear stress on fluid flowing through the microchannel decreasing along the length of the microchannel.

9. The system of claim 1, the capturing agent being covalently immobilized to surfaces of each microchannel with a cross-linker.

10. The system of claim 1, the cross-linker being GMBS.

11. The system of claim 1, further including a pressure pump and a reservoir that is in fluid communication the at least one microchannel, reservoir including a blood sample and the pressure pump configured to provide pressure to reservoir such that the blood sample flows through the at least one microchannel at a physiologically relevant shear stress value.

12. The system of claim 11, wherein the physiologically relevant shear stress value is about 0.5 dyne/cm² to about 1 dyne/cm².

13. The system of claim 1, wherein the imaging system includes a control unit for determining viscosity of the fluid sample.

14. The system of claim 1, where viscosity of the fluid sample is determined by measuring the mean flow velocity of the fluid sample as it passes through the microchannel.

15. The system of claim 1, wherein the endothelial cells are provided in the at least one microchannel by culturing the endothelial on a fibronectin coated surface of the microchannel under continuous flow of the culture medium through the at least one microchannel.

16. A method of measuring efficacy of therapeutic agent in modulating blood cell adhesion; the method comprising:
providing a gas impermeable housing including at least one microchannel defining at least one cell adhesion region, the at least one cell adhesion region being provided with at least one capturing agent that adheres a cell of interest to a surface of the at least one microchannel when a fluid sample containing cells is passed through the at least one microchannel, wherein at least the capturing agent includes at least one of E-Selectin, P-Selectin, intracellular adhesion molecule 1 (ICAM-1), vascular cellular adhesion molecule 1 (VCAM-1) or endothelial cells functionalized to the surface of the microchannel;
perfusing a fluid sample containing the blood cells through the at least one microchannel at a physiologically relevant shear stress rate; and
measuring adherence of the blood cells to the at least one capturing agent when the fluid sample is passed therethrough;
wherein the therapeutic agent is added to at least one of the fluid sample prior to perfusion through the at least one microchannel or the at least microchannel before and/or after the fluid sample is perfused through the at least one microchannel.

17. The method of claim 16, the fluid sample comprising blood and the cells being red blood cells and/or white blood cells.

18. The method of claim 16, wherein the adherence of the blood cells is measured under at least one normoxic or hypoxic conditions.

19. The method of claim 16, wherein the fluid sample is perfused through the microchannel at a physiologically relevant shear stress value.

20. The method of claim 19, wherein the physiologically relevant shear stress value is about 0.5 dyne/cm² to about 1 dyne/cm².

21. The method of 16, wherein the adherence of the blood cells is measured using an imaging system.

22. The method of claim 21, wherein the imaging system includes a control unit for determining viscosity of the fluid sample.

23. The method of claim 22, where viscosity of the fluid sample is determined by measuring the mean flow velocity of the fluid sample as it passes through the microchannel.

24. The method of claim 16, wherein the endothelial cells are provided in the at least one microchannel by culturing the endothelial on a fibronectin coated surface of the microchannel under continuous flow of the culture medium through the at least one microchannel.

25-32. (canceled)
